(19) United States
(12) Patent Application Publication
(10) Pub. No.: US 2021/0032340 A1
(43) Pub. Date: Feb. 4, 2021

(54) METHODS FOR TREATING CANCER WITH BAVITUXIMAB BASED ON LEVELS OF BETA2-GLYCOPROTEIN 1, AND ASSAYS THEREFOR

(71) Applicant: ONCOLOGIE, INC., Waltham, MA (US)

(72) Inventors: Min Tang, Tustin, CA (US); Joseph S. Shan, Tustin, CA (US); Steven W. King, Tustin, CA (US); Connie Chang, Tustin, CA (US); Michael A. Brown, Tustin, CA (US)

(73) Assignee: ONCOLOGIE, INC., Waltham, MA (US)

(21) Appl. No.: 16/336,649

(22) PCT Filed: Sep. 26, 2017
(86) PCT No.: PCT/US2017/053370

§ 371 Date: Mar. 26, 2019

Related U.S. Application Data
(60) Provisional application No. 62/400,549, filed on Sep. 27, 2016.
(60) Provisional application No. 62/406,727, filed on Oct. 11, 2016.
(60) Provisional application No. 62/480,994, filed on Apr. 3, 2017.
(60) Provisional application No. 62/507,580, filed on May 17, 2017.

(51) Int. Cl. C07K 16/28; C07K 16/40; G01N 33/574; A61P 35/00

(52) U.S. Cl. CPC C07K 16/2818; C07K 16/40; A61K 9/0019; A61P 35/00; G01N 33/57484

(57) ABSTRACT

Disclosed are surprising new methods and kits for identifying and treating patients treatable with PS-targeting antibodies, particularly for identifying and treating cancer patients using bavituximab and bavituximab combination therapies. The methods and kits are based on the surprising finding that defined ranges of pre-treatment blood concentrations of β2-glycoprotein 1 (β2GPI), particularly functional β2GPI, act as an indicator to accurately predict patients with better treatment outcomes.

### CROSS-REFERENCE TO RELATED APPLICATIONS

The present application is a U.S. national phase of International Application PCT/US2017/053370, filed Sep. 26, 2017, which claims priority benefit from provisional application Ser. No. 62/507,580, filed May 17, 2017; provisional application Ser. No. 62/480,994, filed Apr. 3, 2017; provisional application Ser. No. 62/406,727, filed Oct. 11, 2016 (Oct. 10, 2016 was a Federal Holiday in the U.S.); and provisional application Ser. No. 62/400,589, filed Sep. 27, 2016, the entire specification, claims, drawings and sequences of which applications are incorporated herein by reference without disclaimer.

### BACKGROUND OF THE INVENTION

### Field of the Invention

The present invention relates to the field of biomarkers, and particularly concerns the use of pre-treatment levels of β2-glycoprotein 1 (β2GPI), particularly functional β2GPI, as an indicator to predict successful responses to therapies using PS-targeting antibodies such as bavituximab.

### Description of the Related Art

In combating all diseases, including cancer and viral infections, a functioning immune system is an important part of a therapeutic response. Significant research has therefore been devoted to immune therapies, including the field of immuno-oncology (IO), which is now recognized as a strategy for treating cancer. In recent years, new targets and compounds that manipulate the immune response have been studied by researchers and clinicians. For example, IO agents that target programmed cell death protein 1 (PD-1) and programmed death-ligand 1 (PD-L1) have already received approval for the treatment of some advanced malignancies, while compounds that interact with other IO targets are in development.

Nonetheless, even these new immunotherapies are only effective in certain patients. Therefore, in light of the variability in response to both long-established therapies and new immunotherapies, and the desire to maximize clinical benefit, there remains a need for biomarkers that can predict treatment outcomes, including to IO therapies. While some researchers are focused on the utilization of the therapeutic target to develop an individualized predictive approach, others are exploring alternative means of patient selection and differentiation by leveraging the measurement of binding and downstream signaling molecules.

Recently, the membrane phospholipid, phosphatidylserine (PS), has been identified as a unique and highly immunosuppressive molecule, which acts as an upstream immune checkpoint that modulates the host immune response. This means that PS plays an important role in various diseases, including cancer and viral infections, opening up a new field of immunotherapeutics in the form of PS-targeting antibodies that block PS.

The lead PS-targeting antibody is bavituximab, a mouse-human chimeric monoclonal antibody (mAb) derived from the murine mAb termed 3G4 (Ran et al., 2005; Huang et al., 2005; U.S. Pat. No. 7,247,303). 3G4 and bavituximab are part of a family of murine, chimeric and fully human antibodies that target PS in a β2-glycoprotein 1 (β2GPI)-dependent manner. That is, bavituximab and related PS-targeting antibodies bind to PS in the presence of β2GPI, such that they form a high affinity antibody-β2GPI-PS complex (Luster et al., 2006). Operationally, these β2GPI-dependent PS-targeting antibodies are specific for PS in vivo, as most particularly shown by numerous imaging studies (Jennewein et al., 2008; Marconescu & Thorpe, 2008; Saha et al., 2010; Stafford & Thorpe, 2011; Zhao et al., 2011; Zhang et al., 2014; and Zhou, et al., 2014; U.S. Pat. No. 7,790,860), including measuring and predicting response to therapy (Gong et al., 2013; Stafford et al., 2013).

Bavituximab has demonstrated activity against a wide range of diseases in which PS is a marker, most particularly cancer and viral infections, but also infections of intracellular parasites, such as the parasitic protozoan, Leishmania amazonensis (Wanderley et al., 2013) and intracellular bacterial pathogens, such as Yersinia pestis and Francisella tularensis, which cause plague and tularemia, respectively (Lonsdale et al., 2011). As to viral infections, PS-targeting antibodies such as bavituximab have been shown to inhibit viral replication, decrease viral load in organs and increase survival (Soares et al., 2008; Moody et al., 2010; U.S. Pat. No. 7,906,115). The anti-cancer activity of bavituximab and related PS-targeting antibodies has been demonstrated in an extensive number of pre-clinical studies and clinical trials, in which effects are mediated against tumor blood vessels as well as by blocking the immunosuppressive signaling of PS (Ran et al., 2005; U.S. Pat. No. 7,572,448; DeRose et al., 2011).

The anti-tumor effects of PS-targeting antibodies such as bavituximab are enhanced when the antibodies are used in conjunction with agents or conditions that increase the exposure of PS in the tumor microenvironment, such as by the use of radiation and/or the co-administration of chemotherapy (U.S. Pat. No. 7,422,738; U.S. Pat. No. 8,486,391; U.S. Pat. No. 7,572,448). For example, improved anti-tumor effects have been demonstrated pre-clinically when using the bavituximab family of PS-targeting antibodies in combination with docetaxel to treat breast tumors (Huang et al., 2005); gemcitabine to treat pancreatic tumors (Beck et al., 2006); irradiation to treat lung cancer (He et al., 2007) and the brain cancer, glioblastoma (He et al., 2009); docetaxel to treat prostate cancer and reactivate antitumor immunity (Yin et al., 2013); and sorafenib to treat hepatocellular carcinoma (Cheng et al., 2016). Enhanced anti-tumor effects also result when PS-targeting antibodies such as bavituximab are used in combination therapies with other IO agents, as shown pre-clinically for the treatment of melanoma (Freimark et al., 2016) and triple-negative breast cancer (Gray et al., 2016a) in combination with checkpoint inhibitors in the form of antibodies to CTLA-4 or PD-1.

Bavituximab has also been evaluated in clinical studies in over 800 patients, most of whom were treated with combination therapies. These clinical trials have included patients with viral infections such as chronic hepatitis C virus (HCV) and human immunodeficiency virus (HIV), and patients with a number of tumor types, including lung, breast, liver (hepatocellular carcinoma, HCC), pancreatic, colorectal and kidney (renal cell carcinoma, RCC). Promising anti-tumor effects have been reported from clinical trials using bavituximab in combination with paclitaxel in patients with HER2 negative metastatic breast cancer (Chalasani et al., 2015); paclitaxel-carboplatin in advanced non-small cell lung cancer, NSCLC (Digumarti et al., 2014); sorafenib in hepatocellular carcinoma (Cheng et al., 2016); and with docetaxel in previously treated, advanced nonsquamous NSCLC (Gerber et al., 2016).

Overall, results from Phase I and Phase II clinical studies demonstrated a clinically meaningful treatment effect of bavituximab. Nonetheless, there have yet to be any biomarkers associated with bavituximab therapy, and so there remains a need for effective methods to optimize treatment with PS-targeting antibodies such as bavituximab. Attempts to address the lack of relevant biomarker data have been hampered by the unique nature of the bavituximab antibody and its physiological/pathological target, PS. Accordingly, there is a need for improved patient screening methods, so that treatment can be optimized. Identifying one or more circulating biomarkers for bavituximab treatment would be a particularly important advance, providing a minimally invasive test to select patients and improve treatment outcomes.

### SUMMARY OF THE INVENTION

The present invention addresses the foregoing and other needs of the prior art by providing new biomarker methods, compositions, kits and assays for optimizing treatment with PS-targeting antibodies such as bavituximab and like antibodies, e.g., 1N11. The invention particularly concerns the use of pre-treatment levels of β2-glycoprotein 1 (β2GPI), most preferably functional β2GPI, as an indicator or “biomarker” to predict successful responses to therapies using bavituximab.

These surprising new methods, compositions, kits and assays provide for identifying and treating patients treatable with β2GPI-dependent PS-targeting antibodies, particularly cancer patients for treatment with a bavituximab-containing therapeutic regimen or combination therapy, most preferably, for identifying and treating patients with cancer using bavituximab (a first anti-cancer agent) and at least a second or third anti-cancer agent, all by selecting patients with defined ranges of pre-treatment blood concentrations of functional β2GPI. “Functional” β2GPI is β2GPI that binds to both PS and to a β2GPI-dependent PS-targeting antibody, preferably bavituximab.

In the most preferred embodiments, the invention concerns the selection, identification, diagnosis and preferably treatment of patients based on pre-treatment levels of functional β2GPI of equal to or greater than 200 μg/ml.

The invention provides a method for treating a disease in which PS is a marker, particularly cancer, in a human patient, comprising administering a PS-targeting antibody, preferably bavituximab, to the patient, wherein the patient has a pre-treatment blood concentration of functional β2GPI of equal to or greater than 200 μg/ml. Preferably, the PS-targeting antibody such as bavituximab, which is herein termed a “first therapeutic agent” and “first anti-cancer agent”, is administered to the patient with at least a second therapeutic or anti-cancer agent.

The invention also provides a method for treating a disease in which PS is a marker, particularly cancer, in a human patient, comprising (the steps of):
(a) measuring the concentration of functional β2GPI in a pre-treatment blood sample obtained from the patient; and
(b) administering a PS-targeting antibody, preferably bavituximab, to the patient with a pre-treatment blood concentration of functional β2GPI of equal to or greater than 200 μg/ml. Preferably, the PS-targeting antibody such as bavituximab is administered to the patient with at least a second therapeutic or anti-cancer agent.

The invention further provides a method for treating a disease in which PS is a marker, particularly cancer, in a human patient, comprising (the steps of):
(a) obtaining a pre-treatment blood sample from the patient;
(b) measuring the concentration of functional β2GPI in the pre-treatment blood sample; and
(c) administering a PS-targeting antibody, preferably bavituximab, to the patient with a pre-treatment blood concentration of functional β2GPI of equal to or greater than 200 μg/ml. Preferably, the PS-targeting antibody such as bavituximab is administered to the patient with at least a second therapeutic or anti-cancer agent.

Also provided by the invention is a method for identifying a human patient, preferably a human cancer patient, treatable with a PS-targeting antibody, preferably bavituximab, and treating the patient, comprising (the steps of):
(a) measuring the concentration of functional β2GPI in a pre-treatment blood sample obtained from the patient;
(b) identifying the patient as treatable with a PS-targeting antibody, preferably bavituximab, when the concentration of functional β2GPI in the pre-treatment blood sample is equal to or greater than 200 μg/ml; and
(c) administering a PS-targeting antibody, preferably bavituximab, to the patient with a pre-treatment blood concentration of functional β2GPI of equal to or greater than 200 μg/ml. Preferably, (b) the patient is identified as treatable with bavituximab and at least a second therapeutic or anti-cancer agent and
(c) bavituximab and at least a second therapeutic or anti-cancer agent is administered to the patient.

Further provided is a method for identifying a human patient, preferably a human cancer patient, treatable with a PS-targeting antibody, preferably bavituximab, and treating the patient, comprising (the steps of):
(a) obtaining a pre-treatment blood sample from the patient;
(b) measuring the concentration of functional β2GPI in the pre-treatment blood sample;
(c) identifying the patient as treatable with a PS-targeting antibody, preferably bavituximab, when the concentration of functional β2GPI in the pre-treatment blood sample is equal to or greater than 200 μg/ml; and
(d) administering a PS-targeting antibody, preferably bavituximab, to the patient with a pre-treatment blood concentration of functional β2GPI of equal to or greater than 200 μg/ml. Preferably, (c) the patient is identified as treatable with bavituximab and at least a second therapeutic or anti-cancer agent and (d) bavituximab and at least a second therapeutic or anti-cancer agent is administered to the patient.

Another embodiment of the invention is a method of diagnosing a patient, preferably a cancer patient, treatable with a PS-targeting antibody, preferably bavituximab, wherein the method comprises measuring the concentration of functional β2GPI in a blood sample from the patient, wherein the patient is determined to be treatable with a PS-targeting antibody, preferably bavituximab, if the blood concentration of functional β2GPI is equal to or greater than 200 μg/ml. Preferably, the patient is diagnosed as treatable with bavituximab and at least a second therapeutic or anti-cancer agent if the blood concentration of functional β2GPI is equal to or greater than 200 μg/ml.

A further embodiment of the invention is a PS-targeting antibody, preferably bavituximab, for use in a method of treating a patient, preferably of treating cancer in a patient, wherein functional β2GPI is present at a concentration of equal to or greater than 200 μg/ml in a blood sample from the patient. Preferably, this concerns bavituximab for use in such a method of treating cancer, further comprising administering at least a second anti-cancer agent.

Yet another embodiment of the invention is a PS-targeting antibody, preferably bavituximab, for use in a method of treating a disease in which PS is a marker, preferably cancer, wherein the method comprises (the steps of):
(a) identifying the concentration of functional β2GPI in a blood sample from the patent, preferably a cancer patient; and
(b) administering a PS-targeting antibody, preferably bavituximab, to the patient if the blood concentration of functional β2GPI is equal to or greater than 200 μg/ml. Preferably, provided are bavituximab and at least a second therapeutic or anti-cancer agent for use in a method of treating cancer.

In addition to the most preferred level of β2GPI of equal to or greater than 200 μg/ml, in certain preferred embodiments, the invention concerns the selection of patients based on pre-treatment functional β2GPI in the range of 200-290 μg/ml. All numbers and ranges within these levels are included in each of the foregoing methods and uses, such as the selection, identification, diagnosis and preferably treatment of patients based on pre-treatment levels of functional β2GPI of equal to or greater than 200 or 210 μg/ml; and β2GPI in the ranges of from any one of 200 or 210 μg/ml as the low number, to any one of 270, 280, 290, 300, 310 or 320 μg/ml as the high number, including 200-270, 200-280, 200-290, 200-300, 200-310, 200-320, 210-270, 210-280, 210-290, 210-300, 210-310 and 210-320 μg/ml and such like, with the ranges 210-270, 210-280, 210-290, 200-280 and 200-290 being currently preferred.

In administering a PS-targeting antibody, preferably bavituximab, to a patient, preferably a human cancer patient, the antibody is given at a dose of between about 1-10, 1-6, 3-6 or 1-3 mg/kg, most preferably, of about 3 mg/kg.

In each of the foregoing methods and uses, the invention is applicable to the use of a PS-targeting antibody, preferably bavituximab with a second or third anti-cancer agent that is a chemotherapeutic agent, such as sorafenib, paclitaxel, carboplatin, gemcitabine or docetaxel, for the treatment of solid tumors, such as ovarian, gastric, liver, colorectal, breast, esophageal, brain (e.g., glioma, glioblastoma), prostate, skin (melanoma), head and neck, kidney, bladder, pancreatic or lung cancer, preferably non-small cell lung cancer (NSCLC), including non-squamous NSCLC. The mechanisms of bavituximab binding in a complex with functional β2GPI and PS, and the immune activating mechanisms of bavituximab overall, are common to all bavituximab therapies. Therefore, the invention applies to the selection of patients for any therapies using PS-targeting antibodies such as bavituximab or like antibodies, e.g., 1N11, particularly in combination therapies, such as with chemotherapy, and preferably with immuno-oncology (IO) agents.

Suitable IO agents are immune checkpoint antibodies, including agonistic (activating) antibodies that bind to an activating immune checkpoint, receptor or molecule, such as CD28, OX40 and/or GITR, and preferably antagonistic (blocking) antibodies that bind to an inhibitory immune checkpoint, receptor or molecule, such PD-1, PD-L1, CTLA-4, TIM-3 and/or LAG-3. Antagonistic (blocking) antibodies that bind to an inhibitory immune checkpoint, receptor or molecule are also herein termed “immune checkpoint inhibitors” or “ICIs”. Preferred examples of immune checkpoint antibodies (or immune checkpoint inhibitors) are blocking antibodies to CTLA-4, PD-1 or PD-L1, such as ipilimumab, tremelimumab, nivolumab, pembrolizumab, durvalumab and atezolizumab.

The invention particularly contemplates the selection, diagnosis and treatment of patients with a PS-targeting antibody, preferably bavituximab, and with a second and third therapeutic or anti-cancer agent. For example, a chemotherapeutic agent and an immune checkpoint antibody, or with two immune checkpoint antibodies, including treatment with a PS-targeting antibody, preferably bavituximab, in which treatment with a chemotherapeutic agent is followed by treatment with an immune checkpoint antibody.

In connection with this β2GPI biomarker technology, and as having other research and clinical uses, the present invention also provides new assay methods, compositions and kits particularly adapted for the detection and quantification of functional β2GPI. Both in terms of the foregoing methods and uses, and as a new assay, the invention therefore further provides methods of measuring functional β2GPI comprising (the steps of):
(a) optionally, coating a solid support such as an ELISA plate with PS to prepare a PS-coated solid support or PS-coated ELISA plate (or a pre-prepared PS-coated solid support or ELISA plate may be used);
(b) adding a PS-targeting antibody, preferably bavituximab, and a biological sample suspected of containing β2GPI to a PS-coated solid support, thereby co-incubating the antibody and sample under conditions effective to allow binding of β2GPI in the sample to both the PS-targeting antibody, preferably bavituximab, and the PS-coated solid support; and
(c) detecting the binding of the PS-targeting antibody, preferably bavituximab, and β2GPI to the PS-coated solid support, thereby measuring the functional β2GPI in the sample.

The biological sample suspected of containing β2GPI may be a blood sample, such as a plasma sample or a serum sample. Other biological fluid samples suspected of containing β2GPI may be used, including cell supernatants and the like.

Many binding formats of the assay may be employed, as disclosed herein and known to those of skill in the art. In preferred embodiments, the PS-targeting antibody, preferably bavituximab, will itself be attached to a detectable agent that produces a detectable signal, such that the binding of the antibody and β2GPI to the PS-coated solid support is detected and measured by detecting and measuring the detectable signal. In other preferred embodiments, the PS-targeting antibody, preferably bavituximab, is added to the PS-coated solid support prior to adding the sample suspected of containing β2GPI, such as a blood sample.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein. The U.S. patent or application file may contain at least one drawing executed in color. Copies of this U.S. patent or patent application publication with color drawing(s) will be provided by the Office upon request and payment of the necessary fee.

FIG. 1A, FIG. 1B, FIG. 1C and FIG. 1D. Inhibition of tumor growth in mice treated with the 3G4 antibody, purified to apparent homogeneity. FIG. 1A, established (0.6-0.7 cm diameter, 140 mm³ volume) human MDA-MB-435 breast carcinomas growing in the mammary fat pads of SCID mice; FIG. 1B, established (0.5-0.7 cm diameter, 110 mm³ volume) human MDAMB-231 breast carcinomas growing in the mammary fat pads of SCID mice; FIG. 1C, small Meth A fibrosarcomas growing subcutaneously in syngeneic BALB/c mice; and FIG. 1D, large (0.8-1 cm diameter, 370 mm³ volume) human L540 Hodgkin's tumors growing subcutaneously in SCID mice. Groups of 8 to 10 mice were injected i.p. with 100 μg of the 3G4 antibody (∘, open circles) or the control, BBG3 antibody (●, closed circles) starting on the day indicated by the arrows. Treatments were continued thrice a week thereafter.

FIG. 2A and FIG. 2B. 3G4 antibody binding to PS-coated microtiter plates is serum-dependent. FIG. 2A, the 3G4 antibody was purified from cells grown in bovine serum-containing media (▴, SCM) or serum-free media (▪, SFM). A microtiter plate was coated with PS and blocked in 1% ovalbumin from chicken egg white (OVA). Serial dilutions of 3G4 were performed in 10% fetal bovine serum (solid lines, FBS) or 1% ovalbumin from chicken egg white (dashed lines, OVA). FIG. 2B, the microtiter plate was coated with PS and blocked in 1% OVA. Serial dilutions of 3G4 in SFM were performed in 10% serum from the species mouse (♦), rat (▪), human (●) and bovine (▴), as indicated.

FIG. 3. The 3G4 antibody binds the plasma protein, β2GPI. A microtiter plate was coated with human β2GPI (hβ2GPI) purified from human plasma and blocked in 1% OVA. Serial dilutions of a commercial mouse anti-human β2GPI (♦, α-β2GPI), the 3G4 antibody from SFM (▪, 3G4) and a control mouse IgG (▴, control mIgG) were performed in 1% OVA.

FIG. 4. The 3G4 antibody binds to β2GPI at domain II. The wells of a microtiter plate were coated with recombinant full-length hβ2GPI (♦, domain I-V) or hβ2GPI peptides without domain I (-X-, domain II-V), without domains I and II (▪, domain III-V), without domains I, II and III (▴, domain IV-V) or without domains I, II, III and IV (●, domain V). The plate was blocked in 1% OVA and serial dilutions of the 3G4 antibody from SFM were performed in 1% OVA.

FIG. 5. The ch3G4 antibody and β2GPI together bind cells with exposed PS. Adult bovine aortic endothelial (ABAE) cells were incubated for 30 min with 200 μM lysophosphatidylcholine (LPC) in DMEM +10% normal mouse serum (MS), plus (i) ch3G4 only, (ii) ch3G4+hβ2GPI simultaneously, or (iii) purified hβ2GPI only. Cells were then washed and incubated for 30 min with (i) buffer only, (ii) buffer only, or (iii) ch3G4, respectively. Finally, cells were washed, fixed, and stained with fluorescent markers to detect binding of ch3G4. ch3G4 and hβ2GPI were used at a concentration of 2 μg/ml. The pixel area of ch3G4 binding was quantified using MetaVue software. Values are relative to the binding of ch3G4 under condition (i), which was set to one.

FIG. 6A and FIG. 6B. The lipid binding region of β2GPI mediates binding of the ch3G4 antibody to cells with exposed PS. FIG. 6A, ABAE cells were incubated with the ch3G4 antibody plus (i) a non-lipid binding form of β2GPI (nicked hβ2GPI) or (ii) intact hβ2GPI (“hβ2GPI”). The incubations were performed in the presence or absence of 200 μM LPC in DMEM +10% MS for 30 min. Cells were then washed, fixed, and stained with fluorescent markers to detect binding of ch3G4. The ch3G4 antibody, intact hβ2GPI and nicked hβ2GPI were used at a concentration of 2 μg/ml. The pixel area of ch3G4 binding was quantified using MetaVue software. Values are relative to the binding of ch3G4 under condition (i) no LPC, which was set to one. FIG. 6B, the wells of a microtiter plate were coated with intact hβ2GPI (triangles, hβ2GPI) or nicked hβ2GPI (squares, nicked) and blocked in 1% OVA. Serial dilutions of the ch3G4 antibody (closed) or a control mIgG (open) were performed in 1% OVA.

FIG. 7A and FIG. 7B. Divalency is required for β2GPI-mediated 3G4 antibody binding to cells with exposed PS. FIG. 7A, ABAE cells were incubated for 30 min with 20 nM 3G4, 3G4 F(ab′)₂ or 3G4 Fab′ monomer in the presence of 200 μM LPC in DMEM +10% FBS. Cells were then washed, fixed, and stained with fluorescent markers to detect binding of the 3G4 antibody or antibody fragments (3G4 F(ab′)₂ and 3G4 Fab′ are shown in FIG. 7A). The pixel area of antibody binding was quantified using MetaVue software. Values are relative to the binding of 3G4 in the absence of LPC, which was set to one. FIG. 7B, ABAE cells were incubated for 30 min with 200 μM LPC, 40 nM purified hβ2GPI, 20 nM ch3G4 and a titer of 3G4 Fab′ monomer in DMEM +10% MS. Cells were then washed, fixed, and stained with fluorescent markers to detect binding of ch3G4. The pixel area of ch3G4 binding was quantified using MetaVue software. Values are relative to the binding of ch3G4 without competing 3G4 Fab′, which was set to 100.

FIG. 8. The 3G4 and bavituximab family of antibodies bind to PS in a β2GPI-dependent manner. 3G4, bavituximab and related therapeutic PS-targeting antibodies bind to β2GPI (at domain II) and β2GPI, in turn, binds to PS via domain V. Normally, β2GPI is monomeric, binds to PS only weakly and rapidly dissociates. In the presence of PS-targeting antibodies such as 3G4 or bavituximab, when there is a surface with exposed PS, two molecules of β2GPI are able to bind to PS and become cross-linked by divalent antibody, such that a stable complex is formed. The antibody-β2GPI complex dissociates from cells with exposed PS more than 1,000-times more slowly than does monomeric β2GPI (in the absence of a PS-targeting antibody).

FIG. 9A and FIG. 9B. Low levels of β2GPI support the pre-clinical anti-tumor activity of the 3G4 antibody in vivo. FIG. 9A, assuming a purity of the 3G4 antibody of 90% when produced from hybridoma cells, mice treated with the 3G4 antibody contain 2 μg/ml of bovine β2GPI at a molar ratio of β2GPI to antibody of 0.12. FIG. 9B, at a starting point of 80% purity, mice treated with the 3G4 antibody contain 4 μg/ml of bovine β2GPI at a molar ratio of β2GPI to antibody of 0.25.

FIG. 10. Low levels of β2GPI support bavituximab binding to cells with exposed PS in vitro. ABAE cells were incubated for 30 min with 200 μM LPC to induce PS exposure, 40 nM purified hβ2GPI, and increasing concentrations of the ch3G4 antibody (bavituximab) in DMEM +10% mouse serum. Cells were then washed, fixed and stained with fluorescent markers to detect binding of ch3G4. The pixel area of ch3G4 binding was quantified using MetaVue software. Values are relative to the binding of 320 pM ch3G4, which was set to one.

FIG. 11. Low levels of β2GPI support 2aG4 antibody binding to PS on plates in vitro. Microtiter plates were coated with PS. Solutions containing a fixed amount of the 2aG4 antibody were tested for binding to PS in the presence of increasing amounts of human β2GPI (in ovalbumin). Bound antibodies were detected using HRP-conjugated anti-human IgG as a secondary antibody, with TMB as a substrate and absorbances were read at a wavelength of 450 nm.

FIG. 12. Bavituximab binding to PS in vitro in a range of human sera. Microtiter plates were coated with PS. Six different individual human serum samples were obtained, nominally termed sera #3 (♦, dark blue line), #4 (▪, pink line), #9 (▴, yellow line), #11 (-x-, light blue line), #13 (-x-, purple line) and #19 (●, brown line), and diluted in PBS to provide a range of % human serum down to 0.1%. Bavituximab-HRP at 2 μg/ml was added to each % human serum solution. Those mixtures of bavituximab-HRP in human sera were added to the PS plates and allowed to bind. Plates were washed, TMB substrate was added and absorbances were read at a wavelength of 450 nm.

FIG. 13. Mean serum bavituximab concentrations following administration to patients with refractory advanced cancer. Bavituximab was administered intravenously at 0.1 mg/kg (∘, open circles) or 0.3 mg/kg (□, open squares) on days 0, 28, 35 and 42; and bavituximab was administered at 1 mg/kg (●, closed circles) or 3 mg/kg (▪, closed squares) on days 0, 7, 14 and 28. The mean serum bavituximab concentrations were determined on the indicated days post-administration. The lower limit of quantitation was 0.1 μg/ml.

FIG. 14. β2GPI levels in patients following bavituximab administration. Repeat doses of bavituximab were administered (on the days indicated by the bold, black arrows, ↓) to patients infected with HCV in amounts of 1 mg/kg, 3 mg/kg and 6 mg/kg. Serum β2GPI levels were determined at the indicated days post-treatment for each dose (1 mg/kg, yellow, top line; 3 mg/kg, pink, middle line; 6 mg/kg, blue, bottom line) and compared to pre-treatment β2GPI levels.

FIG. 15. The 1N11 (PGN635) antibody binds to PS in a serum-dependent manner. Binding of the scFv form of 1N11 was tested by ELISA against plated PS, and a mix of phosphatidylcholine (PC) and sphingomyelin (SM), (PC/SM). Polystyrene plates were coated with 10 μg/ml PS or the same amount of a mix of PC/SM (each dissolved in hexane). After the hexane had evaporated, 10% human serum (+10% serum) or 1% ovalbumin (+1% OV) in PBS was added and incubated for one hour. 20 μg/m1 purified 1N11 scFv was added in either 10% human serum (+10% serum) or 1% ovalbumin (+1% OV) to the first of six wells for each antigen and titrated with 3-fold dilutions. Remaining bound scFv was detected with an HRP-conjugated anti-c-myc tag mouse monoclonal antibody (Invitrogen).

FIG. 16. A standard curve for functional β2GPI. Samples of known amounts of functional β2GPI were tested in the functional β2GPI assay as described in Example XVI and plotted to provide a standard curve. From the standard curve, the amount of functional β2GPI in test samples, particularly diluted plasma or serum test samples, can be determined.

FIG. 17A, FIG. 17B, FIG. 17C and FIG. 17D. Distribution of pre-treatment functional β2GPI levels (μg/ml) in patients participating in a Phase III trial of bavituximab and docetaxel to treat non-small cell lung cancer (NSCLC). FIG. 17A, distribution of functional β2GPI for all 592 evaluable patients; FIG. 17B, Box Plot of β2GPI from the same patients; FIG. 17C, distribution of functional β2GPI for patients treated with bavituximab and docetaxel (294 evaluable patients); and FIG. 17D, distribution of functional β2GPI for patients treated with placebo and docetaxel (298 evaluable patients).

FIG. 18A and FIG. 18B. Kaplan-Meier survival curves from the Phase III trial showing that NSCLC patients having functional β2GPI levels of equal to or greater than 200 μg/ml have a trend for prolonged survival (mOS) when treated with bavituximab. FIG. 18A, in patients treated with bavituximab, those having functional β2GPI≥200 μg/ml (“Bavi: B2GP1>=200”; solid blue, top line) have a trend for prolonged survival as opposed to patients having β2GPI<200 μg/ml (“Bavi: B2GP1<200”; broken blue, bottom line). FIG. 18B, in patients having functional β2GPI≥200 μg/ml, those treated with bavituximab and docetaxel (“Bavi”; blue, top line) have a trend for prolonged survival as opposed to patients having the same β2GPI levels (200 μg/ml or above) treated with placebo and docetaxel (“Placebo”; green, bottom line).

FIG. 19. Comparison of the β2GPI levels that support the PS-binding, functional and anti-tumor activity of the 3G4 antibody in preclinical studies to the β2GPI levels in patients in the Phase III and other clinical trials. The distribution of pre-treatment functional β2GPI levels in 592 evaluable patients in the Phase III trial is the same as set forth in FIG. 17A. Functional β2GPI levels of equal to or greater than 200 μg/ml (dark orange and light orange bars together) provide a trend for prolonged survival of NSCLC patients treated with bavituximab in the Phase III trial (FIG. 18A and FIG. 18B) (and also for other NSCLC and pancreatic cancer patients treated with bavituximab, as in Example XVIII and Example XX). Functional β2GPI levels in the range of between 200 μm/ml and 290 μm/ml (dark orange) provide a statistically significant better mOS for NSCLC patients treated with bavituximab in the Phase III trial (Table 14A). Functional β2GPI levels of about 10 μg/ml or above (→, long green arrow) or about 60 μg/ml or above (>, short green arrow) are sufficient for PS-binding, functional and anti-tumor activities of bavituximab in preclinical studies (Example V).

FIG. 20A, FIG. 20B and FIG. 20C. Distribution of pre-treatment functional β2GPI levels (μg/ml) in patients in the Phase II trial of bavituximab and docetaxel to treat NSCLC of Example XIII, as reported in Example XVIII, A. FIG. 20A, distribution of functional β2GPI for all 119 evaluable patients; FIG. 20B, distribution of functional β2GPI for patients treated with 3 mg/kg bavituximab and docetaxel (40 evaluable patients); and FIG. 20C, distribution of functional β2GPI for patients in the pooled control arm (placebo or 1 mg/kg bavituximab) (79 evaluable patients).

FIG. 21A, FIG. 21B and FIG. 21C. Kaplan-Meier survival curves from the Phase II trial of Example XIII showing that NSCLC patients having functional β2GPI levels of equal to or greater than 200 μg/ml have a trend for prolonged survival when treated with bavituximab. FIG. 21A, in patients treated with bavituximab 3 mg/kg, those having functional β2GPI≥200 μg/ml (“Bavi: B2GP1>=200”; blue, top line) have a trend for prolonged survival as opposed to patients having β2GPI<200 μg/ml (“Bavi: B2GP1<200”; yellow, bottom line). FIG. 21B, in patients having functional β2GPI≥200 μg/ml, those treated with bavituximab 3 mg/kg (“Bavi”; blue, top line) have a trend for prolonged survival as opposed to patients in the combined control arm (“Placebo”; green, bottom line). FIG. 21C, in patients in the combined control arm, those having functional β2GPI≥200 μg/ml (“Placebo: B2GP1>=200”; blue line) have a similar survival to patients having β2GPI<200 μg/ml (“Placebo: B2GP1<200”; yellow line).

FIG. 22. Distribution of pre-treatment functional β2GPI levels (μg/ml) in patients in the Phase II trial of gemcitabine and bavituximab to treat pancreatic cancer of Example XII, as reported in Example XVIII, B. The distribution of functional β2GPI for all 31 evaluable patients is shown.

FIG. 23. Kaplan-Meier survival curve from the Phase II trial of Example XII showing that pancreatic cancer patients treated with gemcitabine and bavituximab have a trend for prolonged survival when functional β2GPI is present at levels of equal to or greater than 200 μg/ml (“B2GP1>=200”; blue, top line) as opposed to functional β2GPI levels of less than 200 μg/ml (“B2GP1<200”; yellow, bottom line).

FIG. 24A, FIG. 24B and FIG. 24C. Distribution of pre-treatment functional β2GPI levels (μg/ml) in patients in the Phase II trial of bavituximab and paclitaxel/carboplatin to treat NSCLC, as reported in Example XVIII, C. FIG. 24A, distribution of functional β2GPI for all 84 evaluable patients; FIG. 24B, distribution of functional β2GPI for patients treated with bavituximab and paclitaxel/carboplatin (44 evaluable patients); and FIG. 24C, distribution of functional β2GPI for patients in the paclitaxel/carboplatin control arm (40 evaluable patients).

FIG. 25A, FIG. 25B and FIG. 25C. Kaplan-Meier survival curves from the Phase II trial reported in Example XVIII, C showing that NSCLC patients having functional β2GPI levels of equal to or greater than 200 μg/ml have a trend for prolonged survival when treated with bavituximab. FIG. 25A, in patients treated with bavituximab, those having functional β2GPI≥200 μg/ml (“C/P+Bavi: B2GP1>=200”; blue, top line) have a trend for prolonged survival as opposed to patients having β2GPI<200 μg/ml (“C/P+Bavi: B2GP1<200”; yellow, bottom line). FIG. 25B, in patients having functional β2GPI≥200 μg/ml, those treated with bavituximab (“C/P+Bavi”; blue, top line) have a trend for prolonged survival as opposed to patients in the control arm (“C/P”; green, bottom line). FIG. 25C, in patients in the control arm, those having functional β2GPI≥200 μg/ml (“C/P: B2GP1>=200”; blue line) have a trend for a reduced survival compared to patients having β2GPI<200 μg/ml (“C/P: B2GP1<200”; yellow line).

FIG. 26. Kaplan-Meier survival curves showing that patients treated with bavituximab and docetaxel followed by subsequent immunotherapy (“SACT-IO”) (blue, top line) have a statistically significant better mOS as opposed to patients treated with docetaxel alone followed by subsequent immunotherapy (green, bottom line). The treatment groups, mOS and statistical analyses are tabulated for these survival curves in Table 16 (Example XIX).

FIG. 27. Kaplan-Meier survival curve showing that NSCLC patients having functional β2GPI levels of equal to or greater than 200 μg/ml have a statistically significant better mOS when treated with bavituximab followed by subsequent immunotherapy (“SACT-IO”). In patients having functional β2GPI≥200 μg/ml, those treated with bavituximab (“Doc+Bavi”, blue lines) have prolonged survival as opposed to control patients (“Doc+Placebo”, green lines), including those receiving SACT-IO (“with SACT IO”, solid lines) and those without SACT-IO (“without SACT IO”, broken lines).

### DETAILED DESCRIPTION OF EMBODIMENTS OF THE INVENTION

In the present era, there is an increasing emphasis on tailoring treatments to the individual patient, based on factors such as their risk of disease and/or predicted response. This concept can generally be described as “personalized medicine”. A greater understanding of different components contributing to the effectiveness of a particular therapy can provide a basis on which to stratify patients, thereby improving treatment outcomes for successive patient populations. The present invention represents an advance along such lines, by providing new biomarkers by which to optimize immunotherapy using PS-targeting antibodies such as bavituximab.

### A. Phosphatidylserine as a Therapeutic Target

Phosphatidylserine (PS) is a highly immunosuppressive molecule that functions as an upstream immune checkpoint and modulates the host immune response. Accordingly, PS is involved in various diseases, including cancer and viral infections. Immunotherapeutic agents in the form of PS-targeting antibodies therefore provide new treatment options for those diseases, including cancer.

In more detail, in normal cells, PS is segregated to the inner leaflet of the plasma membrane, but becomes externalized to the outer leaflet of the plasma membrane in diseased and aberrant cells in various disease states, particularly in cancer and viral infections. In the context of cancer, some of the environmental stressors that cause PS externalization are hypoxia/reoxygenation, oxidative stress and exposure to certain cytokines. PS externalization also occurs under conditions of cell death and immune phagocytic cell clearance (Birge et al., 2016). Subsequently, PS is recognized and bound by PS receptors (e.g., TIM 3 and TIM 4, BAI1, stabilin 2 and RAGE) on immune cells, optionally via one or more of a number of bridging proteins, such that PS induces and maintains immune suppression. In the tumor microenvironment, PS is exposed on the surface of tumor vascular endothelial cells, tumor cells and tumor-derived exosomes, and the process of immune suppression is duplicated, thus preventing antitumor and inflammatory reactions from occurring.

Exposed PS is a phagocytic signal that facilitates the recognition and clearance of dying cells, triggers the release of immunosuppressive cytokines (e.g., TGF-β and IL-10) and inhibits the production of proinflammatory cytokines (e.g., TNF-α and IL-12). PS also polarizes macrophages towards the immunosuppressive M2 phenotype, inhibits dendritic cell (DC) maturation and the ability of DCs to present antigen, while stimulating DCs to secrete immunosuppressive mediators that promote T cell tolerance. In summary, PS is a central factor in the induction and maintenance of an immunosuppressed tumor microenvironment.

### B. PS-Targeting Antibodies

Due to the propensity of PS exposure in the tumor microenvironment to promote tumor progression, PS-targeting antibodies can be used to block the binding of PS to specific receptors on immune cells, and thus provide an effective cancer therapy (Yin et al., 2013). A number of such PS-targeting antibodies have been developed as therapeutics, as exemplified below. The group of “PS-targeting antibodies” includes all antibodies that operatively bind to PS in vitro and specifically localize and bind to PS exposed in disease states in vivo, particularly to PS on tumor cells and tumor blood vessels, irrespective of whether the antibodies bind directly to PS or require a serum protein to form a tight binding complex with PS. Such “direct” and “indirect” PS-targeting antibodies are described in more detail below.

B1. Bavituximab

An early monoclonal antibody generated to evaluate the preclinical potential of PS-targeting antibodies is the antibody termed 3G4, a mouse IgG₃ mAb (Example I; Ran et al., 2005; Huang et al., 2005). Samples of the hybridoma cell line secreting the 3G4 antibody were deposited with the American Type Culture Collection (ATCC) and given ATCC Accession number PTA 4545. Availability of the deposited hybridoma is not to be construed as a license to practice the invention in contravention of the rights granted under the authority of any government in accordance with its patent laws.

Bavituximab is a human chimeric version of the 3G4 mouse antibody, in which the murine variable (antigen binding) regions are operatively attached to a human antibody constant region (Example III, C). The bavituximab family of antibodies is described in detail in numerous U.S. Patents, e.g., U.S. Pat. No. 7,247,303 and U.S. Pat. No. 7,572,448, and bavituximab can be re-created by preparing a human chimeric form of the antibody deposited as ATCC PTA 4545. Bavituximab is less immunogenic when given to patients, because significant portions of the antibody are from human origin.

The 3G4 and bavituximab antibodies bind strongly to anionic phospholipids, particularly PS, but also to phosphatidylinositol (PI), phosphatidic acid (PA), phosphatidylglycerol (PG) and cardiolipin (CL), in the presence of serum (Ran et al., 2005). Of these anionic phospholipids, PS is the most relevant, physiologically and pathologically. 3G4 and bavituximab exhibit no detectable binding to the neutral phospholipids, phosphatidylcholine (PC), sphingomyelin (SM) or phosphatidylethanolamine (PE), irrespective of the presence of serum.

Although it was initially thought that the 3G4 and bavituximab antibodies bound to PS directly, it was later determined that the PS-binding is mediated by a serum protein, which was identified as β2-glycoprotein 1 (β2GPI) (Example IV; Luster et al., 2006). Indeed, 3G4 and bavituximab bind strongly to PS in enzyme-linked immunosorbent assays (ELISAs) conducted in the presence of β2GPI, which includes purified β2GPI as well as β2GPI provided simply by being present in the 10% serum typically used in ELISAs.

β2GPI, also known as apolipoprotein H, has five domains, I, II, III, IV and V (1, 2, 3, 4 and 5), and the domain structure is conserved across mammals. β2GPI folds as a tertiary structure into those five discernable domains, and may have a closed, circular structure or an open, J-shape or hook structure. β2GPI binds to anionic phospholipids, particularly PS, through positively-charged regions in its C terminal domain, domain V, so long as domain V is not “nicked”, such as by cleavage with the enzyme plasmin, at the Lys317/Thr318 cleavage site, which destroys PS binding (Hunt et al., 1993; Hunt & Krilis, 1994). The 3G4 and bavituximab antibodies bind to domain II of β2GPI. This reinforces the safety of 3G4 and bavituximab as therapeutic antibodies, because certain other antibodies that bind to β2GPI have been associated with pathologies, but those antibodies bind to domain I of β2GPI.

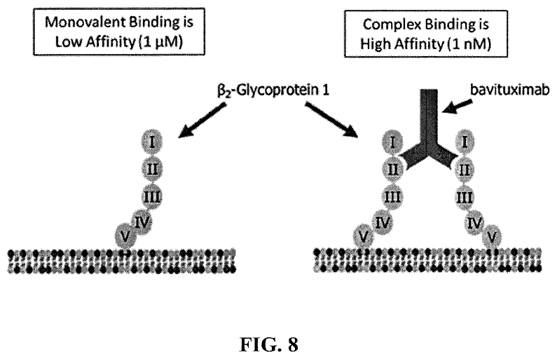

High affinity binding of the 3G4 and bavituximab antibodies to PS requires bivalent interaction of the antibodies with β2GPI (Example IV; FIG. 8). In the absence of such antibodies, β2GPI binds to anionic phospholipids, particularly PS, with only low affinity. This has been quantified in studies showing that 3G4 and bavituximab bind to PS in the presence of β2GPI as a high affinity complex, modulating β2GPI binding to PS from 1 μM to 1 nM.

The β2GPI-dependent binding of the 3G4 and bavituximab antibodies to PS is depicted in FIG. 8. The bavituximab family of antibodies binds to domain II of β2GPI. As mentioned, because bavituximab binds to domain II of β2GPI, it is not linked with side-effects such as those associated with anti-phospholipid syndrome, in which antibodies are present that bind to domain I of β2GPI (de Laat et al., 2005; de Laat et al., 2006; Ioannou et al., 2007). The high affinity bivalent interaction of the antibody with β2GPI coordinates the resultant high-affinity binding to PS, including when PS is externalized on cell surfaces and membranes.

Although the 3G4 and bavituximab antibodies bind to β2GPI, they are referred to as “PS-targeting antibodies” because they specifically localize and bind to PS exposed in disease states in vivo. As PS is maintained on the inside of healthy, normal cells, and only becomes exposed on the cell surface in disease states, antibody localization in vivo is not only specific to PS, but is specific for diseases in which PS is a marker, particularly cancer, but also viral infections and certain other pathologies.

FIG. 8 also shows that β2GPI-dependent antibody binding to PS is the same in vitro as in vivo, such that an ELISA is an accurate model for therapy. In particular, in an ELISA in which the plate is coated with PS and the ELISA is conducted in the presence of serum, the 3G4, bavituximab and like antibodies are able to form a stable binding complex with PS. The ELISA assay therefore mimics the in vivo situation during therapy, in which PS is uniquely exposed only on cells in the disease environment, such as cells in the tumor microenvironment or virally-infected cells. As with the ELISA, when the 3G4 and bavituximab antibodies encounter exposed PS, they are able to form a stable binding complex with the β2GPI present in the blood. Whether the PS is on an ELISA well, or a diseased cell, the antibody-β2GPI complex has more than 1,000-times higher affinity for PS than does monomeric β2GPI, i.e., β2GPI without a PS-targeting antibody.

B2. Direct PS-Binding Antibodies, such as 11.31

In addition to indirect PS-binding or PS-targeting antibodies such as bavituximab, the entire family of PS-targeting antibodies includes antibodies that do bind directly to PS, i.e., direct PS-binding or PS-targeting antibodies. Such a “direct PS-binding antibody” (or “direct PS-targeting antibody”) is an antibody that is not only functionally specific for PS, and targets and binds to PS in vitro and in vivo (as do the indirect binding antibodies), but that does not require a serum protein, such as β2GPI, to form a tight binding complex with PS, even in in vitro binding assays.

One particular example of such a direct PS-binding antibody is the mouse monoclonal antibody termed 9D2 (Ran et al., 2002). The 9D2 antibody has been shown to localize to tumor blood vessels and to exert anti-tumor effects in vivo (Ran et al., 2002). Another example of a direct PS-binding antibody is the fully-human antibody termed 11.31 (PGN632). The 11.31 antibody has also been shown to exert anti-tumor effects in vivo (e.g., in mice bearing MDA-MB-435 mammary carcinoma xenografts) and shows impressive anti-viral effects (Moody et al., 2010; U.S. Pat. No. 7,455,833).

The direct PS-binding antibodies are therefore of use in treating the various diseases in which PS is a marker, most particularly cancer and viral infections. However, biomarkers for optimizing treatment with such direct binding, PS-targeting antibodies will typically not rely on serum proteins, such as β2GPI, as in the present invention, but on other factors. Useful biomarkers for the direct binding antibodies include immune biomarkers for PS-targeting antibodies.

B3. Other β2GPI-Dependent PS-Targeting Antibodies, such as 1N11

The preferred embodiments of the present invention relate to the other part of the PS-targeting antibody family, the indirect PS-binding antibodies. An “indirect PS-binding antibody” or “indirect PS-targeting antibody”, as used herein, is an antibody that is functionally specific for PS, operatively binds to PS in vitro and targets and binds to PS in vivo, but that requires a serum protein to form a tight binding complex with PS. The present invention is particularly concerned with a sub-set of the indirect PS-binding or PS-targeting antibodies, namely the β2GPI-dependent PS-binding or PS-targeting antibodies. A “β2GPI-dependent PS-binding antibody” or “β2GPI-dependent PS-targeting antibody”, as used herein, is an antibody that is functionally specific for PS, operatively binds to PS in vitro, i.e., binds to PS in vitro in assays conducted in the presence of serum containing β2GPI or purified serum, and targets and binds to PS in vivo, but that requires the serum protein, β2GPI to form a tight binding complex with PS. As set forth above, examples of such antibodies include the mouse antibody, 3G4 and the chimeric antibody, bavituximab.

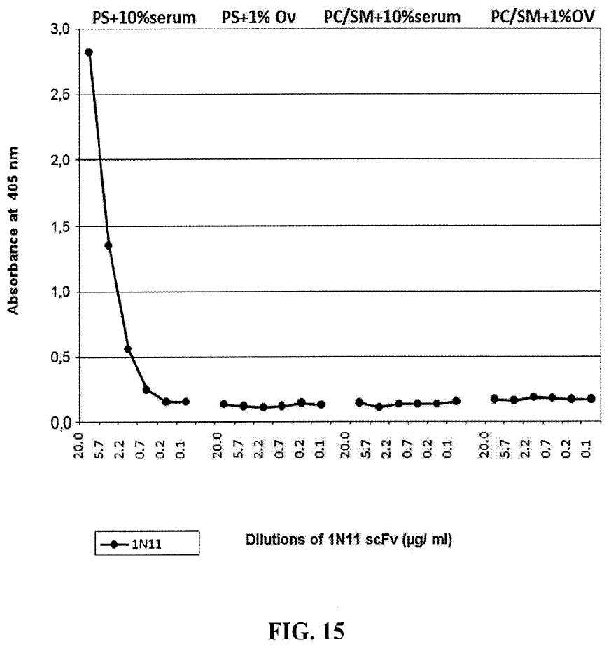

Other currently preferred examples of β2GPI-dependent PS-targeting antibodies are the fully-human antibodies termed 1N11 (PGN635) and 1G15, preferably the 1N11 antibody. Several studies using the 1N11 antibody, and a murine chimeric version thereof, have been described, including imaging and therapy (Gong et al., 2013; Freimark et al., 2016; Gray et al., 2016a). The PS binding properties of the 1N11 antibody are shown in FIG. 15. 1N11 was generated by phage display and selected using assays for binding to PS only in the presence of serum (or only in the presence of β2GP1). As shown by FIG. 15, those of skill in the art can routinely perform such studies to prepare and isolate further PS-targeting antibodies and β2GPI-dependent PS-targeting antibodies.

### C. Extensive Therapeutic Experience

As predicted from the PS biology discussed above, signals from PS inhibit the ability of immune cells to recognize and fight tumors. Bavituximab and related antibodies override this PS-mediated immunosuppressive signaling by blocking the engagement of PS with its receptors, as well as by sending an alternate immune activating signal. PS-targeting antibodies have thus been shown to shift the functions of immune cells in tumors, resulting in multiple signs of immune activation and anti-tumor immune responses.

PS-targeting antibodies such as bavituximab achieve this blocking of PS-mediated immunosuppression by multifocal reprograming of the immune cells in the tumor microenvironment to support immune activation (Yin et al., 2013). Bavituximab and related antibodies thus break immune tolerance in the tumor microenvironment. Antibody-mediated PS blockade reduces the levels of myeloid-derived suppressor cells (MDSCs), transforming growth factor-beta (TGFβ) and interleukin-10 (IL-10), and increases the levels of pro-inflammatory cytokines such as interferon gamma (IFNγ), tumor necrosis factor-alpha (TNFα) and interleukin-12 (IL-12). This PS blockade also repolarizes MDSCs and tumor-associated macrophages (TAMs) from predominant M2 to predominant M1 phenotype, promotes the maturation of dendritic cells (DCs), activates cytotoxic T-cells and induces potent adaptive antitumor T-cell immunity (Yin et al., 2013).

Bavituximab and related antibodies also activate innate immunity, i.e., NK cells as well as M1 macrophages. Importantly, these antibodies also cause the selective shutdown of pre-existing tumor blood vessels, which uniquely expose PS (Ran et al., 2005; U.S. Pat. No. 7,572,448), and this activity includes antibody-dependent cell-mediated cytotoxicity (ADCC) mediated by tumor infiltrating M1 macrophages and NK cells. Destroying the tumor blood vessels in this way leads to tumor cell destruction. These dual mechanisms of immunotherapy and vascular targeting, particularly the ADCC actions, mean that bavituximab can be effective against tumors that are resistant to immune activation or conventional anti-proliferative chemotherapy.

As with other immunotherapeutics, the anti-tumor effects of PS-targeting antibodies are increased when used in combination therapies. One group of agents for use with bavituximab and related antibodies are agents and/or conditions that increase the exposure of PS in the tumor microenvironment, such as radiation and/or chemotherapeutics (U.S. Pat. No. 7,422,738; U.S. Pat. No. 8,486,391; U.S. Pat. No. 7,572,448). Enhanced anti-tumor effects have thus been demonstrated pre-clinically in combination with docetaxel to treat breast tumors (Huang et al., 2005) and prostate cancer (Yin et al., 2013); gemcitabine to treat pancreatic tumors (Beck et al., 2006); irradiation to treat lung cancer (He et al., 2007) and glioblastoma (He et al., 2009); PRIMA-1, which reactivates the mutant tumor suppressor, p53, for advanced breast tumors (Liang et al., 2011); an adenoviral vector to re-target the adenovirus to tumor vasculature (Hogg et al., 2011); cisplatin to treat lung cancer relapse after surgery (Judy et al., 2012); and sorafenib to treat hepatocellular carcinoma (Cheng et al., 2016).

Another group of agents for use with PS-targeting antibodies such as bavituximab are other IO agents. The mechanism of action of bavituximab is complementary to the available therapeutic agents, as PS is an upstream immune checkpoint. Impressive combination therapies have thus been shown pre-clinically for the bavituximab family of antibodies in combination with other checkpoint inhibitors in the form of antibodies to CTLA-4, PD-1 and PD-L1 (Freimark et al., 2016; Gray et al., 2016a). Such anti-tumor activity, which included increased survival, was associated with increases in intratumoral activated CD8 T cells, a reduction of M2 macrophages and MDSCs coupled with PD-L1 expression, and increased tumor reactive T cells in the spleen when compared to PD-1 blockade alone.

Preclinical results such as these therefore confirm that the bavituximab family of PS-targeting antibodies reverses PS-mediated immunosuppression and initiates therapeutically effective adaptive antitumor immunity. Thus, treatment with bavituximab in combination with blockade of downstream immune checkpoints results in robust and long-lasting antitumor immunity that significantly improves outcomes, duration and levels of response (Freimark et al., 2016; Gray et al., 2016a).

In light of the advantageous safety profile of PS-targeting antibodies such as bavituximab, these antibodies may also be effectively combined in triple combination therapies, including triple combinations with radiation, chemotherapeutics (“chemoradiation”) and/or immunotherapeutics, and triple combinations with two immunotherapeutic agents. Impressive results were recently shown for a triple combination using antibodies that target PS, PD-1 and LAG-3 (Gray et al., 2016b).

Building on such preclinical data, bavituximab has been evaluated in clinical studies in over 800 patients, mostly in combination with other indication-approved therapeutics. A range of anti-viral and anti-tumor studies have shown therapeutic activity. Based on extensive pre-clinical work and the pharmacokinetic profile in humans (Example VI; see also, Gerber et al., 2011; Digumarti et al., 2014), a dose of 3 mg/kg bavituximab given intravenously (IV) was determined and selected for most clinical studies in oncology, including in patients with lung, breast, liver, pancreatic, colorectal and kidney cancers. Promising clinical anti-tumor results have now been published, including for bavituximab in combination with: paclitaxel in patients with HER2 negative metastatic breast cancer (Chalasani et al., 2015); paclitaxel-carboplatin in advanced non-small cell lung cancer, NSCLC (Digumarti et al., 2014); sorafenib in hepatocellular carcinoma (Cheng et al., 2016); and with docetaxel in previously treated, advanced nonsquamous NSCLC (Gerber et al., 2016).

In summary, results from the Phase I and Phase II clinical studies demonstrated a clinically meaningful treatment effect of bavituximab. Although there is now a significant body of work showing successful treatment of a range of diseases using PS-targeting antibodies, to date, there are no known biomarkers for such therapies. The clinical experience with PS-targeting antibodies is largely based on the β2GPI-dependent PS-targeting antibodies such as bavituximab. It is for those antibodies that a biomarker to optimize treatment is most needed. If one or more circulating biomarkers for bavituximab treatment could be identified, along with sensitive and rapid methods to quantify such biomarker(s), this would be an important development, providing minimally invasive test(s) to facilitate patient selection for improved treatment outcomes.

### D. Biomarkers for PS-Targeting Antibodies

In the field of cancer therapeutics, biomarkers play an increasingly important role in identifying specific patient characteristics that impact responses to treatment. This has been seen historically with targeted cancer treatments, as well as more recently with checkpoint inhibitors, including PD-1 and PD-L1 inhibitors.

Biomarkers of importance to treatment with PS-targeting antibodies such as bavituximab are being analyzed. As used herein, a “bavituximab biomarker” is a biomarker for use, either alone or as one of two or more, or multiple biomarkers, in selecting patients or patient populations for improved clinical benefit from treatment with therapies that comprise a PS-targeting antibody, preferably bavituximab, as at least part of the therapy. Such bavituximab biomarkers, including β2GPI, may thus be used in methods to predict, in advance of treatment, whether a patient, patient population or sub-population is likely to benefit from a treatment comprising a PS-targeting antibody, preferably bavituximab, including a combination therapy that comprises a PS-targeting antibody, preferably bavituximab.

Multi-marker signatures for identifying the most appropriate patient populations for improved clinical benefit from bavituximab-containing therapeutic regimens are also being considered. The first biomarker identified in these analysis is β2GPI (Section E; Section F). Overall, the pattern of biomarkers identified is a bavituximab “signature” to guide clinical development and treatment.

As part of the bavituximab biomarkers, bavituximab immune biomarkers are being analyzed. Such data support the use of bavituximab to “prime” the immune system, i.e., to amplify anti-tumor immune responses. In this regard, it is now known that tumors can be placed on a scale from “hot” to “cold”, depending on how deeply they have been invaded by T cells and other immune cells. The level of immune infiltration (“heat”) reflects whether the immune system is recognizing and engaging the tumor. Patients with a tumor that is “hot” have a better prognosis; with a “cold” tumor, the probability of relapsing is much higher. Importantly, it has been determined that bavituximab is able to make a positive impact on the cold tumors, making them more amenable to therapy, including with other checkpoint inhibitors. The bavituximab immune biomarkers therefore have additional uses in not only selecting patients for bavituximab therapy, but in identifying patients for treatment with bavituximab and intelligently selected agents for combination therapies.

D1. Samples

For biomarkers other than β2GPI (Section E), the invention may be used to test any biological sample that contains or is suspected to contain one or more of the biomarkers, including any tissue sample or biopsy from an animal, subject or patient, including fecal matter. Clarified lysates from biological tissue samples may be used. However, the invention is preferably used with natural body fluids, thus providing tests that can be performed on biological samples obtained using minimally- or non-invasive techniques, also termed “liquid biopsies”. This is an advantage over more rigorous techniques like biopsies, which typically take longer to provide results and may pose health risks in themselves.

Examples of biological fluids (biofluids) that contain or are suspected to contain one or more biomarkers include blood, urine, ascites, cerebral and cerebrospinal fluid (CSF), sputum, saliva, nasal secretions, bone marrow aspirate, joint or synovial fluid, aqueous humor, amniotic fluid, follicular fluid, cerumen, breast milk (including colostrum), broncheoalveolar lavage fluid, semen, seminal fluid (including prostatic fluid), Cowper's fluid or pre-ejaculatory fluid, female ejaculate, sweat, tears, cyst fluid, pleural and peritoneal fluid or lavage, pericardial fluid, lymph, chyme, chyle, bile, liver perfusate, interstitial fluid, menses, pus, sebum, vomit, vaginal secretions, mucosal secretion, stool water, faecal fluid, pancreatic juice, lavage fluids from sinus cavities, bronchopulmonary aspirates or other lavage fluids. A biological sample may also include the blastocyl cavity, umbilical cord blood or maternal circulation, which may be of fetal or maternal origin.

Preferred examples of biological fluids for testing are blood, urine and ascites fluid, particularly ascites fluid from an animal, subject or patient having or suspected of having ovarian cancer. Where a urine sample is used, it will preferably be used in connection with cancers of the urinary, genitourinary and reproductive systems, such as, e.g., ovarian, prostate, renal, bladder, testicular, urethral and penile cancer. As with β2GPI, detecting and quantifying one or more of the other biomarkers is preferably performed from peripheral blood samples, preferably plasma, and most preferably serum.

D2. PS-Positive Exosomes

Exosomes have recently gained attention in connection with cancer. Exosomes are 40-50 to 100 nanometer (nm) size membrane-derived vesicles that are constitutively released by all cells in vivo and in vitro. Exosomes are biologically active molecular shuttles that play important roles in intercellular communication and influence many physiological and pathological processes. In cancer, exosome functions include the transfer of oncogenes between cancer cells and the tumor stroma that primes the so-called “metastatic niche” for metastatic spread (An et al., 2015).

Due to the multiple intracellular fusion events involved in exosome formation, the luminal contents and proteomic profile of the extracellularly released exosomes mirror those of the originating cell. Thus, tumor-derived exosomes (“tumor exosomes”) have profiles that reflect the cancer cell from which they arose. Indeed, the presence of cytosolic (particularly nucleic acids) and plasma membrane components from the originating cell means that circulating exosomes are readily accessible surrogates that reflect the properties of the parent cell for biomarker analysis. Tumor exosomes, as opposed to exosomes from normal cells, are characterized by having PS on their surface. PS-positive tumor exosomes can thus be used in the diagnosis of cancer. New and improved methods, compositions and kits for diagnosing cancer by detecting and quantifying PS-positive tumor exosomes in biological fluid samples using solid phase assays were recently reported. Such techniques are described in U.S. patent application Ser. No. 15/177,747 and PCT patent application No. PCT/US16/036629, each filed Jun. 9, 2016.

As PS is highly immunosuppressive, the release of PS-positive tumor exosomes is another means by which tumors foster an immunosuppressive environment. Accordingly, the levels of pre-treatment PS-positive tumor exosomes have been proposed for use as a predictive marker for response to therapy for any cancer treatment. Evidently, PS-targeting antibodies need to bind to PS in the disease microenvironment. Therefore, measuring the level of pre-treatment PS-positive tumor exosomes is particularly compelling for use as a predictive biomarker for response to therapy using PS-targeting antibodies such as bavituximab

Methods such as those in U.S. Ser. No. 15/177,747 and PCT Application No. PCT/US16/036629 can thus be used as part of the biomarker tests of the invention. Their combined use with the present quantification of β2GPI, and/or other bavituximab biomarkers may be preferred in certain embodiments, e.g., to enhance the sensitivity of the predictive analyses overall.

### E. β2GPI as a Biomarker

Despite extensive data indicating otherwise (e.g., Example V), the present inventors decided to investigate whether pre-treatment levels of β2GPI could be used as a biomarker, or as part of a panel of biomarkers, to predict treatment outcomes for therapies using bavituximab and related antibodies.

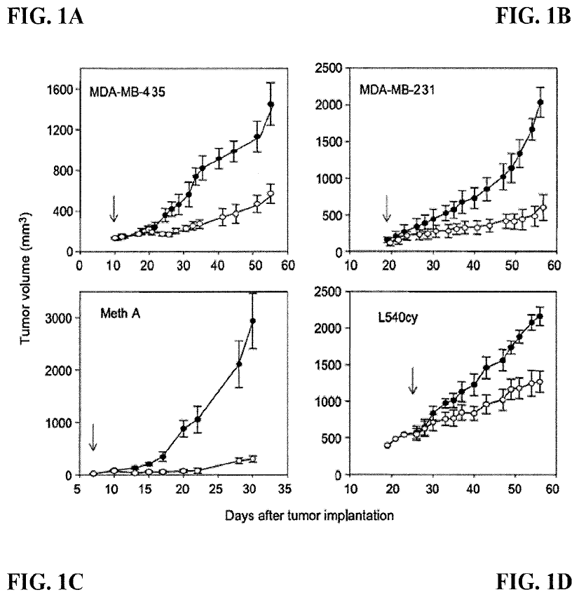

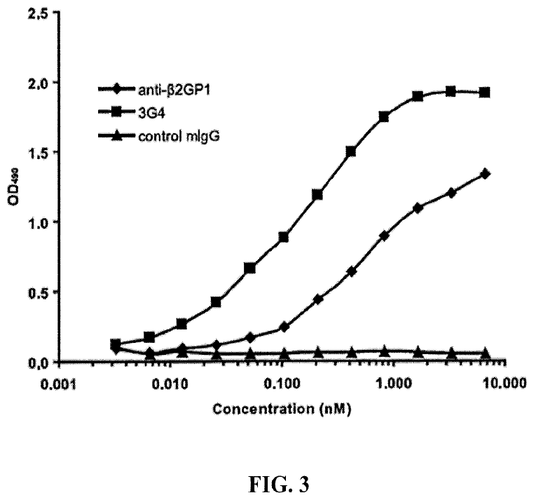

β2GPI is an abundant plasma (serum) glycoprotein found both free and associated with lipoprotein. The DNA and amino acid sequences of β2GPI from various mammalian species are known, including mouse, rat, dog, cow, chimp and human (Steinkasserer et al., 1991). For exemplary reference, the human β2GPI amino acid sequence is provided as Accession number 1C1ZA. β2GPI is glycosylated and is routinely reported as a 50 kDa protein (Example IV, A4,B3; see also, McNeil et al., 1990 at FIG. 3; Balasubramanian et al., 1998 at FIG. 1; Luster et al., 2006 at FIG. 1D). Although β2GPI has been studied for decades, a precise physiological role for β2GPI remains unknown (Prakasam & Thiagarajan, 2012). Indeed, the apparently healthy life of knockout mice deficient in β2GPI indicates that its role is not critical (Sheng et al., 2011).

Surprisingly, it was determined that pre-treatment blood concentrations of β2GPI, particularly functional β2GPI, are effective as a biomarker to predict successful responses to therapies using PS-targeting antibodies such as bavituximab. Indeed, levels of “functional” β2GPI, meaning β2GPI that binds to both PS and to PS-targeting antibodies such as bavituximab, are useful alone as a biomarker for response to bavituximab.

In embodiments of the invention in which pre-treatment β2GPI levels are used alone as a biomarker for response to PS-targeting antibodies such as bavituximab, those β2GPI levels are both numerically defined and measured in assays that are capable of detecting “functional” β2GPI, as defined herein. However, in embodiments of the invention in which pre-treatment β2GPI levels are used as one of two or more, or a plurality, of biomarkers for response to PS-targeting antibodies such as bavituximab, the β2GPI levels need not be so tightly numerically defined, nor solely measured in assays for functional β2GPI.

Accordingly, the β2GPI levels as part of a dual or multi-marker signature for bavituximab-containing therapies can be “β2GPI high” vs. “β2GPI low”, akin to descriptions such as VeriStrat® Good (VS Good) and VS Poor, and tumors that are “hot” or “cold”. Patients with “β2GPI high” should be selected for treatment with PS-targeting antibodies such as bavituximab. In this context, the levels of β2GPI that are “β2GPI high” are pre-treatment levels of β2GPI, either total β2GPI, or preferably functional β2GPI, of equal to or greater than about 180, 190, 200, 210, 220, 230, 240, 250 or 260 μg/ml, preferably of equal to or greater than about 200 μg/ml. “β2GPI high” thus includes pre-treatment levels of β2GPI, either total β2GPI, or preferably functional β2GPI, of equal to about 180, 190, 200, 210, 220, 230, 240, 250, 260, 270, 280, 290, 300, 310 or 320 μg/ml.

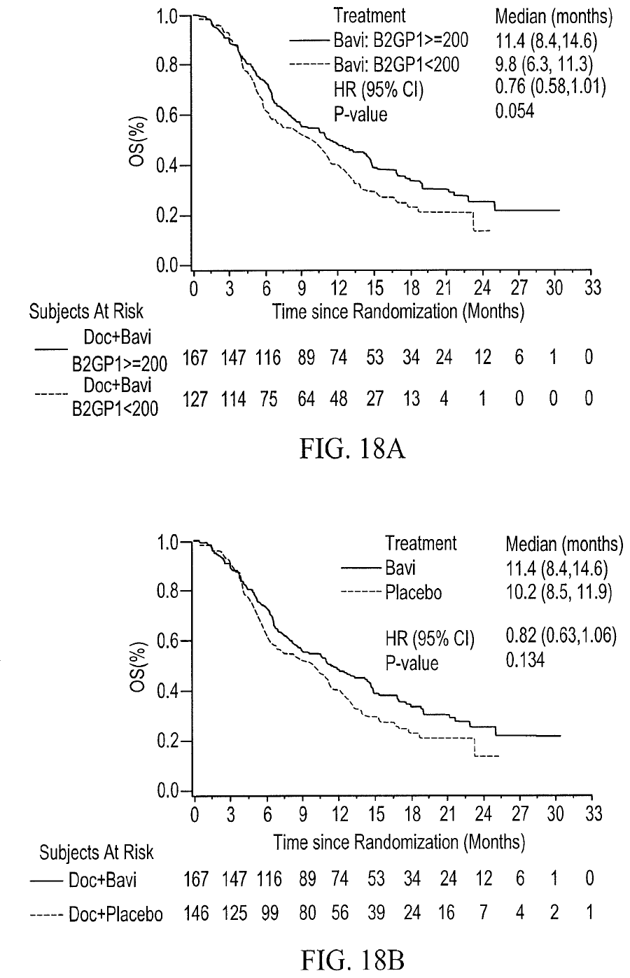

The invention also provides biomarkers in terms of certain numerically defined amounts and ranges of functional β2GPI, measured in assays that are capable of detecting functional β2GPI. In the most preferred embodiments, the invention concerns the selection and treatment of patients based on pre-treatment levels of functional β2GPI of equal to or greater than 200 μg/ml (Example XVII; FIG. 18A and FIG. 18B; Example XVIII; Example XX). This may include pre-treatment levels of functional β2GPI of equal to about 200, 210, 220, 230, 240, 250, 260, 270, 280, 290, 300, 310 or 320 μg/ml.

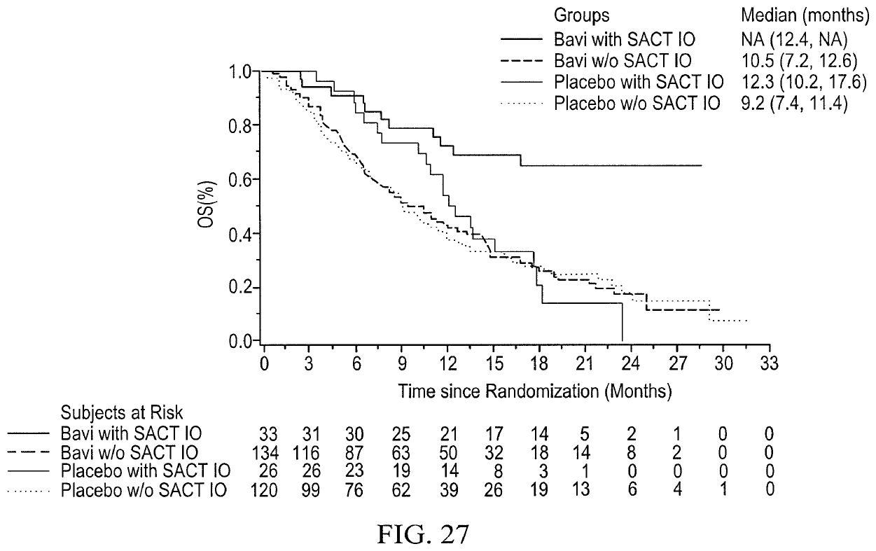

As exemplified by the data in Example XVII, Example XVIII and Example XX, bavituximab improved overall survival in patients with functional β2GPI of equal to or greater than 200 μg/ml in all clinical trials from which β2GPI data are available. The results are summarized in Table A, in which the Treatment column refers to bavituximab treatment of the listed indication in combination with the recited agent. In addition, for NSCLC patients treated with bavituximab and subsequent IO (Example XX), functional β2GPI of equal to or higher than 200 μg/ml was shown to provide a survival advantage for bavituximab over placebo (mOS not reached for bavituximab vs. 12.3 months for placebo; p=0.002; FIG. 27), although a comparison between the bavituximab-treated patients in the β2GPI≥200 and β2GPI<200 groups cannot yet be made because mOS has yet to be reached for either group.

TABLE A
Pre-Treatment β2GPI ≥ 200 μg/ml Improves
Bavituximab Overall Survival

| | | Median Overall Survival (months) | | |
|---|---|---|---|---|
| Treatment | N | β2GPI ≥ 200 | β2GPI < 200 | P-value |
| NSCLC | 294 | 11.4 | 9.8 | 0.054 |
| Docetaxel | | | | |
| NSCLC | 40 | 16.8 | 9.4 | 0.564 |
| Docetaxel | | | | |
| NSCLC | 44 | 17.0 | 14.2 | 0.147 |
| paclitaxel/carboplatin | | | | |
| Pancreatic Cancer | 31 | 7.4 | 5.3 | 0.038 |
| Gemcitabine | | | | |

As well as the widely applicable level of β2GPI of equal to or greater than 200 μg/ml, certain currently preferred embodiments of the invention concern the selection and treatment of patients based on pre-treatment levels of functional β2GPI in the range of 200-290 μg/ml (Example XVII; compare Table 14A and Table 14B), particularly for treating NSCLC. This also includes pre-treatment levels of functional β2GPI in the ranges of 200-270, 200-280, 200-290, 200-300, 200-310, 200-320, 210-270, 210-280, 210-290, 210-300, 210-310 and 210-320 μg/ml and such like, with the ranges 210-270, 210-280, 210-290, 200-280 and 200-290 being currently preferred.

In further embodiments, the invention concerns the selection and treatment of patients based on pre-treatment levels of functional β2GPI in the ranges of from any one of about 190, 200, 210 or 220 μg/ml as the low number, to any one of about 260, 270, 280, 290, 300, 310 or 320 μg/ml as the high number. These ranges include all the following, from within which, the ranges of about 210-270, 210-280, 210-290, 200-280 and 200-290 μg/ml are preferred:

about 190-260, 190-270, 190-280, 190-290, 190-300, 190-310 and 190-320;

about 200-260, 200-270, 200-280, 200-290, 200-300, 200-310 and 200-320;

about 210-260, 210-270, 210-280, 210-290, 210-300, 210-310 and 210-320; and

about 220-260, 220-270, 220-280, 220-290, 220-320, 220-310 and 220-320.

In addition to the most preferred and generally applicable selection and treatment of patients based on pre-treatment levels of functional β2GPI of equal to or greater than 200 μg/ml, whichever one or more of the above numbers or ranges are chosen for any particular treatment or combination treatment, the use of pre-treatment levels of β2GPI, preferably functional β2GPI, as a biomarker, or as part of a panel of biomarkers, applies to the selection of patients with a wide range of diseases in which PS is a marker, most particularly cancer and viral infections, but also infections of intracellular parasites, and their treatment using any PS-targeting antibody, such as bavituximab, either alone, or preferably in any combination therapy.

### F. Assays for β2GPI

As pre-treatment levels of β2GPI are a biomarker for bavituximab and related antibodies, the following guidance is provided concerning assays for β2GPI. The present invention also provides certain preferred assays for quantifying functional β2GPI (Section G).

F1. β2GPI Samples

As a serum protein, β2GPI is ideal for detection in peripheral blood (plasma, serum) samples, as described below. However, studies have suggested that under various pathophysiologic conditions in which PS is involved, β2GPI localizes to endothelial cells in vivo (Agostinis et al., 2011). Therefore, the full range of biological samples (Section D1) can potentially be used for β2GPI detection.

Nonetheless, peripheral blood, plasma and serum samples are particularly preferred for detecting and quantifying β2GPI, whether total β2GPI or functional β2GPI (Section G). Whole blood may be used (red blood cells, white blood cells, platelets, proteins and plasma). Preferably, plasma is used, which is the liquid remaining after the precipitation of red cells and white cells. Plasma contains fibrinogen and other clotting factors, so tends to clot on standing. Less clot-prone plasma is available, which is preferred; platelet-free plasma may also be used. Most preferably, serum is used for detecting and quantifying β2GPI. Serum is plasma without the clotting factors, mainly without fibrinogen, so serum does not clot on standing. Animal and human sera are routinely used for diagnostic purposes, and preparative techniques are widely known. Exemplary methods for preparing serum samples for β2GPI testing are shown herein (Example XV, A).

It is an advantage of the invention that the tests may be carried out directly on the biological sample, preferably blood, plasma or serum. Due to the sensitivity, β2GPI can readily be detected without any prior enrichment or concentration (although this is not excluded). The test samples, preferably serum samples, may be fresh or previously frozen and then thawed. Example XV, Example XVI, Example XVII and Example XVIII show that β2GPI is stable to long-term storage at −70° C. Industry-standard techniques of freezing, storage and/or thawing should preferably be used, such as using cryogenic tubes or vials and/or protease inhibitors to limit proteolysis overall.

F2. Range of β2GPI Assays

The breadth of assays for measuring β2GPI without reference to whether it is “functional” β2GPI, i.e., assays for “total” β2GPI, are applicable for use with those embodiments of the invention in which the pre-treatment β2GPI levels are used as only one of two or more biomarkers for bavituximab. Where levels of β2GPI are used alone as a biomarker for bavituximab, a “functional” β2GPI assay should be used, such as described in Section G.

Total β2GPI levels may be detected and preferably quantified using any one or more of the many in vitro binding assays and kits known in the art. Suitable binding assays include, for example, immunoblots, Western blots, dot blots, RIAs, immunohistochemistry, fluorescent activated cell sorting (FACS), immunoprecipitation, affinity chromatography, and the like. Although solid phase binding assays are typically preferred, various other methods for detecting β2GPI have been described in the literature, any of which may be used. For example, β2GPI levels may be accurately determined by radial immunodiffusion. Indeed, radial immunodiffusion has been used to quantify β2GPI from the late 1960s to more contemporary times (e.g., Balasubramanian et al., 1998). Isoelectric focusing (IEF) followed by immunoblotting may also be used to quantify β2GPI (Kamboh et al., 1988), as may Western blotting, immunoelectrophoresis and Ouchterlony double immunodiffusion (Takeuchi et al., 2000).

F3. Solid Phase β2GPI Binding Assays

Numerous sensitive, solid phase binding assays for β2GPI are known in the art and total β2GPI will preferably be detected and quantified using one or more of such assays. A preferred example of such an assay is as an enzyme linked immunosorbent assay (ELISA). Various ELISAs specific for total β2GPI have been reported in the literature, including modified capture ELISAs (e.g., Mehdi et al., 1999) and competitive ELISAs (e.g., Balasubramanian et al., 1998). Numerous commercial kits for assaying total β2GPI are available, as are commercially available anti-β2GPI antibodies, including those attached to diagnostic labels. Any such kits or antibodies may be used to detect and quantify total β2GPI. For example, anti-β2GPI antibodies from US Biological are used herein in comparative assays (Example XVI, A10,B2).

In general terms, ELISAs for total β2GPI use one or more anti-β2GPI antibodies. Even though antibody technology is very advanced, the commercial kits and commercial anti-β2GPI antibodies often use polyclonal anti-β2GPI antibodies, which are completely suitable for use in such embodiments. In an exemplary assay for total β2GPI, anti-β2GPI antibodies are adsorbed to a solid surface, such as a 96 well plastic plate, and incubated with the biological sample suspected of containing β2GPI (in this case, the “antigen”). Bound β2GPI (antigen) is detected using a secondary binding agent that is directly or indirectly labeled with a detectable agent, i.e., an agent that produces a detectable signal, such as color or fluorescence, which can be detected and quantified. Preferably, the secondary binding agent is an anti-β2GPI antibody that is labeled with a detectable agent.

Such ELISAs for total β2GPI are exemplified in Example XVI, A10,B2 and many general components and steps, such as solid supports and detectable agents, are also described more fully below in terms of the functional β2GPI assay of the present invention (Section G). Therefore, unless evident that particular reagents or steps apply only to use in the functional β2GPI assay, their application in assays for detecting total β2GPI is contemplated herein.

### G. Preferred ELISA for Functional β2GPI

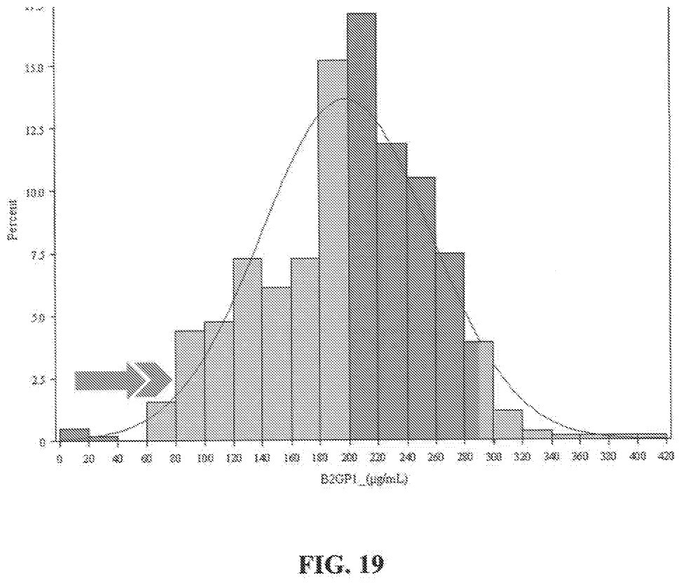

Although various commercial assays and research tools are available to analyze clinical trial results for biomarkers to predict better outcomes, none were known to be uniquely applicable to PS-targeting antibodies such as bavituximab. Despite the extensive pre-clinical modelling and significant prior clinical experience indicating that low and/or varying levels of serum β2GPI would not significantly impact treatment outcomes for bavituximab (Example V; FIG. 19), an analysis of the β2GPI concentrations in the patients from the Phase III trial (Example XIV) was sought.

However, reliable and quantitativeβ2GPI assays to specifically detectβ2GPI that can bind to PS, as opposed to totalβ2GPI, were not available. Such an assay is necessary for the most precise measurements as applied to biomarkers, particularly because it is well known that a portion of β2GPI (“total” β2GPI) will exist as nicked β2GPI, which cannot bind to PS and thus cannot mediate antibody binding in the disease site. Moreover, there was a marked lack of any assay to specifically detect β2GPI that can bind not only to PS, but also to PS-targeting antibodies such as bavituximab. This is particularly important for the highest fidelity biomarker measurements, e.g., to rule out the possibility that β2GPI with other meaningful changes was being detected, particularly mutations and/or nicking or cleavage in, or impacting, domain II, as any such β2GPI alterations would diminish or negate antibody binding and the formation of the antibody: β2GPIPS complex required for therapeutic activity.

Therefore, in order to conduct the optimal analyses of β2GPI concentrations in patients treated (or to be treated) with PS-targeting antibodies such as bavituximab, including patients from the Phase III trial (Example XIV), it was necessary to first invent a new assay. The present application discloses such an advantageous assay, which is uniquely adapted for the purpose of detecting and quantifying the amount of functional (active) β2GPI in human blood samples, such as plasma and serum, which assay can determine the level of β2GPI that is able to bind to both PS and to PS-targeting antibodies such as bavituximab.

It is by use of such an assay for functional β2GPI that the present invention provides defined levels of pre-treatment β2GPI for use as a single biomarker for response to treatment with bavituximab and related PS-targeting antibodies. Notably, functional β2GPI of equal to or greater than 200 μg/ml (Example XVII; FIG. 18A and FIG. 18B; Example XVIII; Example XX) is broadly predictive of response to treatment with bavituximab, as exemplified by functional β2GPI in the range of 200-290 μg/ml (Example XVII; compare Table 14A and Table 14B). The preferred ELISAs for functional β2GPI provided by the invention are exemplified by the detailed teaching in Example XVI and are also described more fully below.

G1. Assay Methods

In general terms, solid phase assays such as ELISAs for functional β2GPI use both PS and a PS-targeting antibody such as bavituximab, at least one of which is operatively associated with a solid support and/or at least one of which is directly or indirectly labeled with a detectable agent. All binding formats can be used. For example, the PS-targeting antibody could be adsorbed to the solid support and the PS labeled with a detectable agent. Many lipids such as PS labeled with detectable agents are known in the art, any of which could be used. However, for simplicity, the currently preferred embodiments are those in which PS is adsorbed to a solid surface, such as a 96 well plastic plate. This means that the PS-targeting antibody, such as bavituximab or 1N11, can be labeled with a detectable agent, which is preferably a direct label attached to the antibody.

In these assays, the PS-coated solid support, such as an ELISA plate (or wells thereof), is incubated with the PS-targeting antibody, such as bavituximab or 1N11, and the biological sample suspected of containing β2GPI. Conceptually, it is important that the PS-targeting antibody, preferably bavituximab, and the β2GPI samples are “co-incubated” on the PS-coated solid support or plate. Scientifically, the β2GPI can either bind to PS via (intact) domain V, followed by bavituximab binding to domain II of the β2GPI bound to the plate; or bavituximab can bind to domain II of the β2GPI in solution, followed by the complexed bavituximab/2GPI (with an intact domain V) binding to the PS on the plate. Both binding events occur during the incubation time, so co-incubating encompasses all such mechanisms of binding.

“Co-incubating” the PS-targeting antibody, preferably bavituximab, and the β2GPI samples on the PS-coated support thus means incubating together under “effective binding conditions”, i.e., “under conditions and for a time effective to allow binding of β2GPI in the sample to both the PS-targeting antibody and the PS-coated support”. “Binding” means under conditions and for a time effective to allow “specific binding”, i.e., binding that is not removed by routine washing. In using bavituximab, the effective binding conditions thus permit binding of PS to intact (non-nicked) domain V of β2GPI and binding of bavituximab to domain II of β2GPI. In using a PS-targeting antibody other than bavituximab, the effective binding conditions permit binding of PS to intact (non-nicked) domain V of β2GPI and binding of the PS-targeting antibody to β2GPI at a domain of β2GPI other than domain V, preferably to the hinge region joining domains I and II, and most preferably to domain II of β2GPI.

So long as the PS-targeting antibody, preferably bavituximab, and the β2GPI samples are co-incubated on the PS-coated solid support or plate, the assays of the invention include a number of different formats. For example, the PS-targeting antibody and the sample suspected of containing β2GPI may be added to the PS-coated support substantially simultaneously. Preferably, the PS-targeting antibody and the sample suspected of containing β2GPI are added to the PS-coated support sequentially, i.e., at times spaced apart.

In performing sequential assay binding steps, the PS-coated solid support, such as an ELISA well, may first be incubated with the biological sample suspected of containing β2GPI and then co-incubated with the PS-targeting antibody, preferably bavituximab. The above assay may utilize a pre-prepared PS-coated support, in which case the assay comprises (or comprises the steps of):
(a) adding the biological sample suspected of containing β2GPI to a PS-coated support under conditions effective to allow binding of β2GPI in the biological sample to the PS-coated support via an intact domain V of β2GPI, thereby preparing a PS- and β2GPI-coated support;
(b) adding the PS-targeting antibody, preferably bavituximab, to the PS- and β2GPI-coated support under conditions effective to allow binding of the PS-targeting antibody to the PS- and β2GPI-coated support by antibody binding to a β2GPI domain other than domain V, preferably to allow binding of bavituximab to the PS- and β2GPI-coated support by antibody binding to β2GPI domain II; and
(c) detecting the binding of the PS-targeting antibody, preferably bavituximab, to the PS- and β2GPI-coated support, thereby measuring the functional β2GPI in the biological sample.

However, in performing sequential assay binding steps, it is preferred that the PS-targeting antibody, preferably bavituximab, and most preferably detectably-labeled bavituximab, is applied to the PS-coated solid support first, followed by co-incubating with the biological sample suspected of containing β2GPI. Such an assay, or sequence of steps, is preferred for technical reasons, e.g., to avoid cross-contamination during pipetting. Such a preferred assay comprises (or comprises the steps of):
(a) coating a solid support with PS to prepare a PS-coated support;
(b) adding the PS-targeting antibody, preferably bavituximab, and most preferably detectably-labeled bavituximab, to the PS-coated support to prepare an antibody-lined PS-coated support;
(c) adding, essentially without washing, the biological sample suspected of containing β2GPI to the antibody-lined PS-coated support under conditions effective to allow binding of β2GPI in the biological sample to the antibody-lined PS-coated support, i.e., by binding of β2GPI to the PS-coated support via an intact domain V of β2GPI and by binding of β2GPI to the PS-targeting antibody via a β2GPI domain other than domain V, preferably by binding of β2GPI to bavituximab via β2GPI domain II; and
(d) detecting the binding of the PS-targeting antibody, preferably bavituximab (most preferably detectably-labeled bavituximab), and β2GPI to the PS-coated support, thereby measuring the functional β2GPI in the biological sample.

This assay may also utilize a pre-prepared PS-coated support, in which case the assay comprises (or comprises the steps of):
(a) adding the PS-targeting antibody, preferably bavituximab, and most preferably detectably-labeled bavituximab, to a PS-coated support to prepare an antibody-lined PS-coated support;
(b) adding, essentially without washing, the biological sample suspected of containing β2GPI to the antibody-lined PS-coated support under conditions effective to allow binding of β2GPI in the biological sample to the antibody-lined PS-coated support, i.e., by binding of β2GPI to the PS-coated support via an intact domain V of β2GPI and by binding of β2GPI to the PS-targeting antibody via a β2GPI domain other than domain V, preferably by binding of β2GPI to bavituximab via β2GPI domain II; and
(c) detecting the binding of the PS-targeting antibody, preferably bavituximab (most preferably detectably-labeled bavituximab), and β2GPI to the PS-coated support, thereby measuring the functional β2GPI in the biological sample.

The preferred assay with sequential binding steps, in which the PS-targeting antibody, preferably bavituximab, and most preferably detectably-labeled bavituximab, is applied to the PS-coated solid support first, followed by co-incubating with the biological sample suspected of containing β2GPI, succinctly comprises (or comprises the steps of):
(a) coating a solid support with PS to prepare a PS-coated support;
(b) adding a PS-targeting antibody, preferably bavituximab, and most preferably detectably-labeled bavituximab, and a biological sample suspected of containing β2GPI to the PS-coated support under conditions effective to allow binding of β2GPI in the sample to both the PS-targeting antibody, preferably bavituximab, and the PS-coated support; preferably, wherein the PS-targeting antibody is added to the PS-coated support prior to adding the sample containing β2GPI and wherein they are co-incubated together; and
(c) detecting the binding of the PS-targeting antibody and β2GPI to the PS-coated support, thereby measuring the functional β2GPI in the sample.

Again, the assay may utilize a pre-prepared PS-coated support, in which case the assay comprises (or comprises the steps of):
(a) adding a PS-targeting antibody, preferably bavituximab, and most preferably detectably-labeled bavituximab, and a biological sample suspected of containing β2GPI to a PS-coated support under conditions effective to allow binding of β2GPI in the sample to both the PS-targeting antibody, preferably bavituximab, and the PS-coated support; preferably, wherein the PS-targeting antibody is added to the PS-coated support prior to adding the sample containing β2GPI and wherein they are co-incubated together; and
(b) detecting the binding of the PS-targeting antibody and β2GPI to the PS-coated support, thereby measuring the functional β2GPI in the sample.

Bound PS-targeting antibody and β2GPI (antigen) is detected using at least a secondary binding agent in the form of at least a PS-targeting antibody, preferably bavituximab or 1N11, which is directly or indirectly labeled with a detectable agent. An unlabeled PS-targeting antibody can be used in connection with a tertiary binding agent, preferably another antibody, which binds to the PS-targeting antibody and that is directly labeled with a detectable agent. Such tertiary binding antibodies are well-known in the art and, e.g., specifically bind to the Fc portion of the PS-targeting antibody.

For simplicity, the currently preferred embodiments are those in which the PS-targeting antibody, preferably bavituximab, is itself directly attached to the detectable agent. The detectable agent is an agent that produces a detectable signal, such as color or fluorescence, which can be detected and measured or quantified. An exemplary detectable agent is the enzyme horseradish peroxidase (HRP), wherein the HRP cleaves the substrate 3,3′5,5′ tetramethylbenzidine (TMB) to produce a colored signal that is detected and measured at 450 nm. Typically, the quantity of bound material measured from the signal is compared to the level of a “reference signal”, such as a standard curve. If desired, a standard curve can be replicated in every assay.

In all formats of these assays, the only β2GPI that is ultimately detected is β2GPI capable of binding to both PS and to the PS-targeting antibody, i.e., β2GPI that is not removed overall by routine washing. These assays are therefore uniquely suited for detecting pre-treatment β2GPI in the form most relevant to clinical treatment, i.e., β2GPI that “functions” to form a binding complex with the administered antibody, preferably bavituximab, and the PS exposed in the disease site, preferably in the tumor microenvironment. The use of these assays is therefore advantageous in the selection of patients for improved treatment outcomes on bavituximab therapy.

The functional β2GPI assays provided by the invention are also simple, reproducible, sensitive, cost-effective and ideal for use with biological samples obtained by minimally invasive techniques, particularly blood (serum and plasma) samples. The rapid nature of the assays provides the important advantage that the biomarker test can be performed quickly and treatment decisions made and implemented in a timely manner. However, the new assays for quantifying functional β2GPI provided by the invention are not limited solely to use in measuring β2GPI for use as a biomarker in a therapy using a PS-targeting antibody such as bavituximab. As β2GPI is an important molecule in basic and directed pre-clinical research and in clinical studies, these assays may be used to measure functional β2GPI in any one or more such embodiments, e.g., such as in connection with knock-out mice or antiphospholipid syndrome (APS) in humans.

G2. Solid Supports

The solid phase binding assays of the invention typically require operatively associating the binding constructs with a solid support or substrate (which has at least one surface for coating or attachment). “Binding constructs”, as used herein, include constructs that bind to components useful in the detection of biomarkers. In connection with the β2GPI biomarker, binding constructs include anti-β2GPI antibodies, PS and PS-targeting antibodies such as bavituximab.

Such solid supports or substrates include, e.g., plates, beads and fibers. In preferred embodiments of the invention, the solid support or substrate is a multi-well plate, such as a standard 96-well plate. The solid support or substrate may be fabricated from any suitable material, such as sepharose, latex, glass, polystyrene, polyvinyl, nitrocellulose, silicon, silica, polydimethylsiloxane (PDMS) and the like. The binding construct is operatively associated with the solid support or substrate by effectively contacting at least one surface of the support or substrate with the binding construct. Preferably, the binding construct is immobilized on at least one surface of the solid support or substrate. The binding constructs can also be printed onto coated glass slides and used in biomarker arrays or microarrays. Both non-contact and contact printing can be used to prepare such microarrays, with contact printing being preferred.

G3. Detectable Agents

Suitable detectable agents include, e.g., enzymes, such as horseradish peroxidase (HRP), alkaline phosphatase (AP), β-galactosidase and urease. A horseradish-peroxidase detection system can be used, for example, with the chromogenic substrate tetramethylbenzidine (TMB), which yields a soluble product in the presence of hydrogen peroxide that is detectable at 450 nm. Other convenient enzyme-linked systems include, for example, the alkaline phosphatase detection system, which can be used with the chromogenic substrate p-nitrophenyl phosphate to yield a soluble product readily detectable at 405 nm. Similarly, a β-galactosidase detection system can be used with the chromogenic substrate O-nitrophenyl-β-D-galactopyranoside (ONPG) to yield a soluble product detectable at 410 nm, or a urease detection system can be used with a substrate such as urea-bromocresol purple.

Further examples of detectable agents include chemiluminescent labels and labels for fluorescent detection. Useful fluorochromes include DAPI (4′,6-diamidino-2-phenylindole), fluorescein, Hoechst 3325S, R-phycocyanin, B-phycoerythrin, R-phycoerythrin, rhodamine, Texas red and lissamine. Fluorescein or rhodamine labeled antibodies or annexins, and/or fluorescein- or rhodamine-labeled secondary antibodies can be used. Isotopes can also be useful in the detection methods, which moieties and assays are well known in the art.

The detectable agent produces a detectable signal, which is then detected and preferably quantified. A detectable signal can be analyzed, e.g., using a spectrophotometer to detect color from a chromogenic substrate; a radiation counter to detect radiation, such as a gamma counter for detection of ¹²⁵I; or a fluorometer to detect fluorescence in the presence of light of a certain wavelength. Where an enzyme-linked assay is used, quantitative analysis of the detectable signal can be performed using a spectrophotometer.

G4. Kits

The invention also provides a series of biomarker-based kits, including diagnostic, prognostic and predictive therapy kits. The biomarker kits will typically comprise one or more of the binding constructs useful in the detection of the biomarkers taught herein. Kits in connection with the β2GPI biomarker will generally comprise at least a first β2GPI binding construct, such as anti-β2GPI antibodies, PS and PS-targeting antibodies such as bavituximab.

Other kits will comprise both binding constructs for biomarker detection and at least a first therapeutic agent for use in treating a selected patient, e.g., a PS-targeting antibody such as bavituximab or 1N11, or an immunoconjugate thereof. Such kits may further comprise at least a second or third distinct therapeutic agent for use in combination treatment with the PS-targeting antibody. For example, one or more chemotherapeutic, radiotherapeutic, anti-angiogenic, immunotherapeutic and/or anti-viral agents.

In general, the kits will contain the stated components in at least a first suitable container (or container means). The containers will generally include at least one vial, test tube, flask, bottle, syringe or other container or container means, into which the desired agents are placed and, preferably, suitably aliquoted. The kits will also typically include a means for containing the individual vials, or such like, in close confinement for delivery, such as, e.g., injection or blow-molded plastic containers into which the desired vials and other apparatus are placed and retained.

The components of the kits may be contained either in aqueous media or in lyophilized form. When reagents or components are provided as a dry powder, the powder can be reconstituted by the addition of a suitable solvent. The solvent may also be provided in another container within the kit. Any therapeutic components will preferably be in a pharmaceutically acceptable formulation, or ready for reconstitution as such. The kits may also contain a means by which to administer the therapeutic agents to an animal or patient, e.g., one or more needles or syringes, or an eye dropper, pipette, or other such like apparatus, from which the formulations may be injected into the animal or applied to a diseased area of the body.

The kits will preferably have distinct containers for each desired component or agent, particularly the biomarker detection and diagnostic components. However, for use in combined therapies, the kits may comprise one container that contains two or more therapeutics, pre-mixed; either in a molar equivalent combination, or with one component in excess of the other. The kits may include pre-labeled antibodies in fully conjugated form, or separate label moieties to be conjugated by the user of the kit, preferably with instructions for attachment. For immunodetection, one or more of the components, such as PS, may already be bound to a solid support, such as a well of a microtitre plate.

The kits will preferably also include written or electronic instructions for use, e.g. in quantification, pre-clinical, clinical and/or veterinary embodiments, including for use in combined therapy. Being biomarker-based, the kits will preferably further comprise control agents, such as suitably aliquoted biological compositions, whether labeled or unlabeled, as may be used to prepare a standard curve for a detection assay.

G5. Chip and Nano Assay Formats

The solid phase and ELISA-type biomarker assays, including for total β2GPI and/or functional β2GPI, can be automated or performed robotically, if desired, and the signal from multiple samples can be detected simultaneously. Various such assay formats have been used to detect and quantify biomarkers in general, although not in the context of the present invention. For example, nano-plasmonic sensors and microfluidic devices termed “Chips” have been described and used for on-chip isolation, quantification and characterization of circulating biomarkers from cancer patients. The present assays can thus be accomplished using such microfluidic, chip, nano-tech and other streamlined and automated assays, whilst still retaining the specificities of the invention.

In addition to the predictive methods and biomarker-guided treatment methods, the present invention also provides computer-based hardware and tests. Such computer-based embodiments of the invention include an interface configured for reading one or more laboratory biomarker tests, including for total β2GPI and/or functional β2GPI, and a computer programmed to analyze data from such biomarker tests and, preferably, to compare the analyzed data to established data sets, including test data sets and control data sets. The computer-implemented embodiments of the invention will preferably include memory storage, output functions and instructions configured to guide therapy based upon the output.

### H. Disease Treatment

As the present invention provides biomarker methods, compositions and kits for selecting animals and humans and optimizing treatment with PS-targeting antibodies such as bavituximab, the following guidance concerning animals, subjects and patients, including human patients, applies to both the biomarker detection and the treatment of the selected population.

H1. Animals, Subjects and Patients

The invention is most directly applicable to human subjects and patients, such that the selection and treatment of humans are the most preferred embodiments. Nonetheless, the commonality and conservation of the biomarkers across species means that invention is applicable to animals other than humans. Within animals, mammals are preferred, most preferably, valued and valuable animals such as domestic pets, race horses and animals used to directly produce (e.g., meat) or indirectly produce (e.g., milk) food for human consumption, although experimental animals are also included. The invention therefore includes clinical, veterinary and research uses. In addition to humans, the invention therefore applies to dogs, cats, horses, cows, pigs, boar, sheep, goat, buffalo, bison, llama, deer, elk and other large animals, as well as their young, including calves and lambs, and to mice, rats, rabbits, guinea pigs, primates such as monkeys and other experimental animals.

H2. Antibody Doses

A “therapeutically effective” amount or dose of a PS-targeting antibody such as bavituximab is an amount or dose that exerts a beneficial therapeutic effect when administered to an animal, preferably a human patient, in need of such a therapy, including when administered as part of a combination therapy. For example, a therapeutically effective anti-cancer dose is an amount or dose that exerts a beneficial anti-cancer effect when administered to an animal, preferably a human patient, with cancer, including when administered as part of a combination cancer therapy. A therapeutically effective anti-viral dose is an amount or dose that exerts a beneficial anti-viral effect when administered to an animal, preferably a human patient, with a viral infection or disease, including when administered as part of a combination viral therapy.

“Beneficial anti-cancer effects” include any consistently detectable anti-tumor and anti-cancer effect, including tumor vasculature thrombosis and/or destruction, tumor necrosis, tumor regression and tumor remission, up to and including cures. Clinical measures of beneficial anti-cancer effects include, for example, improvements in overall response rate (ORR), including complete response (CR), partial response (PR), and CR+PR; time to tumor progression (TTP); duration of response (DOR or DR); and improvements or extensions in progression-free survival (PFS), disease-free survival (DFS) and overall survival (OS), including median overall survival (mOS), in individual patients, patient populations and sub-populations, as applicable.

“Beneficial anti-viral effects” include any consistently detectable anti-viral effect, including inhibiting viral infection, replication, maturation, reproduction and egress and/or ongoing infection of, or spread to, additional cells (host cells) or tissues. Clinical measures of beneficial anti-viral effects include, for example, early virological response, reductions in viral load and clearance of virus, as well as improvements in the symptoms caused by the viral infection.

It will be understood that beneficial therapeutic effects, particularly anti-cancer effects, may not be curative, particularly in the intermediate or long term, but that does not negate the usefulness of the therapies. In this regard, but also in general, “beneficial” therapeutic, anti-cancer and anti-viral effects also include comparative and/or modest treatment effects, but with improvements in any one or more measures of safety. Another consideration for “beneficial” therapeutic effects is the fact that the PS-targeting antibodies such as bavituximab may predispose the disease or tumor to further therapeutic treatment, such that a subsequent treatment can result in an overall improved effect.

Therapeutically effective doses of PS-targeting antibodies such as bavituximab or 1N11 are now readily determinable using a wide range of data, including from animal models, but particularly based on clinical studies, such as those detailed herein, and published in the literature. In general, the effective dose ranges of PS-targeting antibodies such as bavituximab, given intravenously (IV) and quoted in mg/kg, will be between about 0.1 and about 13-15, preferably between about 0.1 and about 6-10; preferably, between about 0.3 and about 6; more preferably, between about 0.5 and about 6; more preferably, between about 1 and about 6; more preferably, between about 0.5 and about 3 or between about 3 and about 6;

more preferably, between about 1 and about 3. Exemplary effective doses of PS-targeting antibodies such as bavituximab, given IV and quoted in mg/kg, will be about 1, 2, 3, 4, 5, 6, 7, 8, 9, 10, 11, 12, 13, 14 and about 15; preferably about 0.1, 0.3, 0.5, 1, 1.5, 2, 2.5, 3, 3.5, 4, 4.5, 5, 5.5 and about 6; more preferably, about 2 or 3; and most preferably, of about 3 mg/kg.

The currently preferred dose of 3 mg/kg bavituximab given intravenously (IV) for clinical treatment, particularly for all oncology indications, is recommended based on extensive pre-clinical and clinical data, and particularly on the pharmacokinetic profile in humans (Example VI), along with the extensive safety data. Nonetheless, a range of doses have been shown to be effective, including clinical anti-viral activity at 0.3 mg/kg (Example VI). In addition, bavituximab has been safely administered to rats and monkeys at doses above 10 mg/kg, up to 100 mg/kg. At the 100 mg/kg dose level in monkeys, bavituximab transiently decreased β2GPI in the systemic circulation, so such ultra-high doses are not recommended.

Therefore, from the breadth of data, it is evident that the dose of 3 mg/kg, although preferred, is not limiting on the invention. Accordingly, it will be understood that, given the parameters and detailed guidance presented herein, further variations in the active or optimal dose ranges and doses will be encompassed within the present invention. It will thus be understood that lower doses may be more appropriate in combination with certain agents, and that high doses can still be tolerated, particularly when treating a usually fatal disease.

In administering PS-targeting antibodies such as bavituximab, a pharmaceutically acceptable composition (according to FDA standards of sterility, pyrogenicity, purity and general safety) is administered to the animal or patient systemically. Intravenous injection is generally preferred, and a continuous infusion over a period of several hours is most preferred.

In addition to varying the dose itself, the administration regimen can also be adapted to optimize the treatment strategy, as is well known to those of skill in the art. Some variation in dosage and treatment regimen may be necessary depending on the condition of the subject being treated. The physician(s) responsible will, in light of the present disclosure, be able to determine the appropriate treatment for the individual subject. Such optimization and adjustment is routinely carried out in the art, and by no means reflects an undue amount of experimentation.

H3. Supplementing Treatment with β2GPI

In using pre-treatment β2GPI levels as a biomarker for response to PS-targeting antibodies such as bavituximab, either alone, or as part of a multi-biomarker selection, and irrespective of whether total β2GPI or functional β2GPI is measured, the methods will select only a sub-set of patients for treatment.

Another embodiment of the present application is thus to restore any non-selected patients to treatment eligibility by co-administering β2GPI to those patients along with the PS-targeting antibody such as bavituximab. In this manner, the entire population becomes treatable with PS-targeting antibodies. For example, in selecting patients for treatment based on pre-treatment levels of functional β2GPI of equal to or greater than 200 μg/ml, a patient with a pre-treatment level of functional β2GPI of 150 μg/ml could be returned to the treatable group by co-administering bavituximab in conjunction with sufficient functional β2GPI to restore the β2GPI levels to at least about 200 μg/ml. The sample applies to whichever pre-treatment level of β2GPI is used in the biomarker analyses.

I. Treating Diseases in which PS is a Marker

As PS-targeting antibodies such as bavituximab specifically target PS, the first, and most important, indication for treatment is cancer (Section K), particular solid tumors and their metastases, but also liquid tumors, such as leukemias, and preferably, Hodgkin's Lymphoma.

In normal and healthy cells, PS is maintained on the inside of the cell membrane and not accessible to binding. Only cells in diseases have PS exposed on the outside of the cell membrane, most particularly, cells in the tumor microenvironment, but also dying cells, aberrant cells, inappropriately activated cells, infected cells and pathogenic organisms themselves. In cancer, PS exposure in the tumor microenvironment is “immunosuppressive”, meaning that the body cannot adequately fight the cancer. By blocking PS, bavituximab overrides the PS-mediated immunosuppression, helping the body attack the tumor.

In cells in the tumor microenvironment, most particularly cells lining the blood vessels in tumors (and in virally-infected cells and viruses), PS is a relatively stable marker, meaning that it is an ideal target for therapy. In diseases where there is a lot of cell death, PS is also exposed on the outside of cells, which means that bavituximab can be used in diagnosis and particularly for “imaging”, i.e., in vivo diagnostics, of a variety of diseases in which increased or inappropriate cell death occurs, including such conditions as, e.g., cancer and heart attacks, (see below for imaging).

Prominent pathogens that cause the host cell to externalize PS are viruses (Section J). Indeed, the role of PS and PS receptors as enhancers of enveloped virus entry and infection is now well-documented and applies to a wide range of viruses. Moreover, the connection between PS and viruses is not limited to enveloped viruses, but extends to non-enveloped viruses. In particular, it is known that “PS lipid vesicles” released from virally-infected cells enable efficient en bloc transmission of enteroviruses (Chen et al., 2015).

In addition to cancer and viral infections, a wide range of diseases and pathogenic infections cause PS to flip from its interior location in healthy cells to become exposed on the outside of the cell, meaning that PS-targeting antibodies such as bavituximab can localize to those cells and pathogens and exert beneficial effects. Collectively, these are “diseases and disorders in which PS is a marker”.

Other than cancer, viral and pathogenic infections, prominent diseases and disorders in which PS is a marker are diseases in which aberrant vasculature (blood vessels) is involved, including diseases and disorders having prothrombotic blood vessels (prone to clotting) and those involving aberrant angiogenesis. Angiogenesis is the process through which new blood vessels form from pre-existing vessels; the development of new blood vessels begins with the formation of endothelial cell sprouts, which requires PS (Weihua et al., 2005). Aberrant angiogenesis is involved in many diseases, most notably in cancer. In light of their aberrant vasculature, PS-targeting antibodies such as bavituximab can treat benign (as opposed to malignant) tumors, such as benign prostatic hyperplasia (BPH), acoustic neuroma, neurofibroma, trachoma, granulomas including pyogenic granulomas and sarcoidosis (sarcoid), meningioma, angiofibroma, angioma, hemangiomas and systemic forms of hemangiomas, the hemangiomatoses.

Conditions directly associated with aberrant vasculature that can be treated with PS-targeting antibodies such as bavituximab include vascular restenosis (narrowing of blood vessels), including restenosis following angioplasty, vein occlusion, artery occlusion and carotid obstructive or occlusive disease; vasculitis (disorders that destroy blood vessels by inflammation), including Behcet's disease (also an eye disease), polyarteritis nodosa (panarteritis nodosa or PAN) and Wegener's granulomatosis (WG) or sarcoidosis (granulomatosis with polyangiitis, GPA); arteriovenous malformations (AVM) and arteriovenous fistula; epistaxis (nosebleeds); vascular adhesions; and hyperviscosity syndromes.

Due to their connection with aberrant vasculature, PS-targeting antibodies such as bavituximab can treat clinically important diseases including joint diseases such as arthritis, including rheumatoid arthritis and osteoarthritis, synovitis, hemophilic joints and Paget's disease; skin diseases such as psoriasis, dermatitis, scleroderma (systemic sclerosis or CREST syndrome), pseudoxanthoma elasticum (PXE, known as Gronblad-Strandberg syndrome), rosacea, Stevens-Johnson syndrome or disease (PXE, rosacea and Stevens-Johnson syndrome are also eye diseases), pemphigoid, hypertrophic scars and keloids; Grave's disease; endometriosis; and Osler-Weber (or Osler-Weber-Rendu) syndrome or disease (also known as hereditary hemorrhagic telangiectasia, HHT).

Particularly important examples of diseases involving aberrant vasculature to be treated by PS-targeting antibodies such as bavituximab are ocular neovascular diseases. These diseases are characterized by invasion of new blood vessels into the structures of the eye, such as the retina, choroid and/or cornea. They are the most common cause of blindness and are involved in approximately twenty eye diseases. The most common ocular neovascular diseases are (proliferative) diabetic retinopathy, macular degeneration, including age-related macular degeneration (AMD), retinopathy of prematurity (ROP or Terry syndrome, previously known as retrolental fibroplasia, RLF), neovascular glaucoma, corneal graft neovascularization and corneal graft rejection. Choroidal neovascularization (CNV) accounts for 90% of cases of severe vision loss in patients with advanced AMD, and has been effectively treated with PS-targeting antibodies, including both direct and indirect PS-targeting antibodies (Li et al., 2015).

Other diseases associated with retinal/choroidal neovascularization that can be treated with PS-targeting antibodies such as bavituximab include syphilitic, mycobacterial and/or other eye infections causing retinitis or choroiditis; uveitis (iridocyclitis), including vitritis and pars planitis; Eales disease, presumed ocular histoplasmosis syndrome (POHS), Best's disease (vitelliform macular dystrophy), Stargardt disease, eye trauma and post-laser complications.

Further diseases particularly associated with corneal neovascularization that can be treated with PS-targeting antibodies such as bavituximab include all forms of keratoconjunctivitis, including keratitis (only the cornea is inflamed) and conjunctivitis (only the conjunctiva is inflamed), such as atopic keratitis, superior limbic keratitis, pterygium keratitis sicca and marginal keratolysis; phylectenulosis; Mooren ulcer; chemical burns, bacterial ulcers, fungal ulcers, Herpes infections and traumas of the eye and contact lens overwear.

Other ocular diseases that can be treated with PS-targeting antibodies such as bavituximab include scleritis, rubeosis (neovascularization of the iris), neovascularization of the angle (NVA), and diseases caused by the abnormal proliferation of fibrovascular or fibrous tissue, including all forms of proliferative vitreoretinopathy (PVR), whether or not associated with diabetes.

The formation of endothelial cell sprouts requires PS, so the development of new blood vessels also requires PS (Weihua et al., 2005). This process is also involved in certain normal physiological events, particularly wound healing and reproduction, and is important in ovulation and in the implantation of the blastula after fertilization. Prevention of this process using bavituximab can thus be used to induce amenorrhea (absence of a menstrual period in women of reproductive age), to block ovulation and/or to prevent implantation by the blastula, i.e., as a contraceptive. In wound healing, excessive repair or fibroplasia can be a detrimental side effect of surgical procedures and adhesions are a frequent complication of surgery, which can lead to problems such as small bowel obstruction. These can also be treated by PS-targeting antibodies such as bavituximab.

Chronic inflammation also involves aberrant and pathological vasculature. In particular, chronic inflammatory disease states such as ulcerative colitis and Crohn's disease show histological changes with the ingrowth of new blood vessels into the inflamed tissues. Those diseases can thus also be treated by PS-targeting antibodies such as bavituximab.

Several other diseases and disorders are known in which the host cells expose PS and/or in which PS-positive extracellular microvesicles and exosomes have been documented. For example, in sickle cell disease (also called sickle cell anemia) and crisis, 30-40% of erythrocytes are prematurely senescent and PS-positive (“sickle erythrocytes”), as opposed to only about 1% in healthy people. The PS-positive sickle erythrocytes remain in circulation, adhere to the endothelium and their exposed PS acts as a platform for the initiation of the coagulation cascade that is responsible for clot propagation (Kennedy et al., 2015).

PS is also expressed in atherosclerosis and PS-positive extracellular microvesicles are released from atherosclerotic plaques (Mallat et al., 1999). The plaques formed within the lumen of blood vessels, which are positive for PS, have also been shown to have angiogenic stimulatory activity. There is particular evidence of the pathophysiological significance of angiogenic markers, such as VEGF, in the progression of human coronary atherosclerosis, as well as in recanalization processes in obstructive coronary diseases. PS-targeting antibodies such as bavituximab thus provide an effective treatment for atherosclerosis and obstructive coronary diseases.

Both Type 1 and Type 2 diabetic patients have PS-positive extracellular microvesicles, as shown by being annexin V-positive (Sabatier et al., 2002). In Alzheimer's disease, brain exosomes contain PS and amyloid β-peptide (Aβ), the pathogenic agent of the disease (Yuyama et al., 2012). PS and PS-positive extracellular microvesicles are also involved in sepsis (septic shock), where they are markers and mediators of sepsis-induced microvascular dysfunction and immunosuppression (Souza et al., 2015).

Antiphospholipid syndrome (APS) and systemic lupus erythematosis (SLE or lupus), autoimmune disorders in which antibodies are produced against the body's own phospholipids, are associated with coagulation disorders, including miscarriages and thrombocytopenia (low platelet counts). Accordingly, the anti-phospholipid antibodies in these patients are pathogenic antibodies, which cause thrombosis. PS-targeting antibodies such as bavituximab, however, target PS without exhibiting any such side effects. Accordingly, bavituximab can also treat antiphospholipid syndrome, associated diseases and complications thereof. In particular, bavituximab can antagonize or compete with the pathogenic antibodies in APS patients, thus displacing the pathogenic antibodies from their phospholipid-protein targets in the body.

As to pathogenic infections, for example, intracellular parasites, such as the parasitic protozoan, Leishmania amazonensis, which causes leishmaniasis (Zandbergen et al., 2006; Wanderley et al., 2009; Wanderley et al., 2013); Plasmodium falciparum, which causes malaria (Eda & Sherman, 2002; Pattanapanyasat et al., 2010); and Trypanosoma cruzi, a parasitic protozoan that causes trypanosomiasis (DaMatta et al., 2007), all result in PS exposure. Likewise, Schistosoma, parasitic flatworms that cause schistosomiasis, also expose PS (van der Kleij et al., 2002), as does Toxoplasma gondii, which causes toxoplasmosis (Seabra et al., 2004).

PS exposure has also been shown on the exterior cell surface following infection by intracellular bacterial pathogens, such as Yersinia pestis and Francisella tularensis, which cause plague and tularemia, respectively (Lonsdale et al., 2011). Listeria monocytogenes, which causes listeriosis, also promotes the release of membrane-derived vesicles with exofacial PS from infected host cells (Czuczman et al., 2014). Similarly, endothelial cells infected with the meningitis-causing pathogen, Neisseria meningitidis, exhibit PS translocation to the cell surface (Schubert-Unkmeir et al., 2007). Infection with Mycobacterium tuberculosis, which replicates intracellularly in macrophages and causes tuberculosis (TB), is associated with PS externalization in neutrophils in the tubercle lesion (Francis et al., 2014). Likewise, Legionella pneumophila, a facultative intracellular parasite that causes Legionnaires' disease, induces PS externalization in human monocytes (Hägele et al., 1998).

Thus, the PS externalization common to the facultative intracellular parasites detailed above is likely to occur for other such pathogens, such as Brucella and Salmonella, which cause brucellosis and illnesses such as typhoid fever, paratyphoid fever and food poisoning, respectively. This has also been documented for infection by obligate intracellular parasites, such as Chlamydia spp., which cause sexually transmitted chlamydia infections, in which PS externalization is important to pathogenesis and has been shown on infected epithelial, endothelial, granulocytic and monocytic cells (Goth & Stephens, 2001). Chlamydia trachomatis can also be treated, which causes trachoma (also see above).

Indeed, PS externalization on host cells is now a generally recognized phenomenon in response to infection with a range of bacteria and pathogens (Wandler et al., 2010). This further includes Helicobacter pylori, which invades gastric epithelial cells (Petersen & Krogfelt, 2003) and causes stomach ulcers. When H. pylori has direct contact with gastric epithelial cells, it induces externalization of PS to the outer leaflet of the host plasma membrane (Murata-Kamiya et al., 2010). PS is also present on Treponema pallidum, which causes syphilis. Bartonellosis, a bacterial infection found in South America, can be treated with bavituximab, particularly because bartonellosis results in a chronic stage that is characterized by proliferation of vascular endothelial cells, and one of bavituximab's mechanisms of action, as clearly shown in cancer treatment, is to destroy vascular endothelial cells.

With reference to in vivo diagnostics, PS-targeting antibodies such as bavituximab may be used for imaging any of the foregoing diseases, disorders and infections, most preferably, for imaging vascularized tumors (Jennewein et al., 2008; Marconescu & Thorpe, 2008; Saha et al., 2010; Stafford & Thorpe, 2011; Gong et al., 2013; Stafford et al., 2013; U.S. Pat. No. 7,790,860). Bavituximab may also be used for imaging vascular thromboses, particularly in or near the heart, such as in deep vein thrombosis, pulmonary embolism, myocardial infarction, atrial fibrillation, problems with prosthetic cardiovascular materials, stroke (cerebrovascular accident (CVA) or cerebrovascular insult (CVI)), and the like. PS-targeting antibodies such as bavituximab may also be used in imaging activated platelets, e.g., in conditions such as abscesses, restenosis, inflammation of joints and in hemostatic disorders, such as arterial, coronary, venous and cerebral thrombosis and such like.

PS-targeting antibodies such as bavituximab are thus suitable for treating and/or diagnosing all the above diseases and disorders, in which PS is a documented marker.

### J. Treating Viral Infections

Prominent pathogens that cause the host cell to externalize PS are viruses. The presence of PS has been demonstrated on the surface of viruses and virally-infected cells and/or shown to be important to infections from a wide range of viral families, including Arenaviridae, Bunyaviridae, Flaviviridae, Filoviridae, Herpesviridae, Orthomyxoviridae, Paramyxoviridae, Poxviridae, Retroviridae and Rhabdoviridae (see also, Table 2A and Table 2B in U.S. patent application Ser. No. 14/634,607 and PCT patent application No. PCT/US15/18183, each filed Feb. 27, 2015). In addition, data have been presented to demonstrate that such PS exposure on viruses and virally-infected cells is not merely incidental, but has an important role in viral infections (see also, Table 2C and Table 2D in U.S. patent application Ser. No. 14/634,607; U.S. Pat. No. 7,906,115; WO 2015/131153 A1). This is shown by the use of PS-targeting antibodies to inhibit infections from diverse viral families, both in vitro and in vivo.

The connection between PS and viral infections is also now well documented in the literature (e.g., U.S. Pat. No. 7,906,115; Soares et al., 2008; Mercer and Helenius, 2008; Moody et al., 2010; Morizono et al., 2011; Meertens et al., 2012; Best, 2013; Bhattacharyya et al., 2013; Jemielity et al., 2013; Moller-Tank & Maury, 2014; Birge et al., 2016). This includes the role of PS and PS receptors as enhancers of enveloped virus entry and infection (see, e.g., Table 1 in Moller-Tank & Maury, 2014). The relationship between PS, viral infections and extracellular microvesicles such as exosomes has also become increasingly apparent in recent years (Meckes & Raab-Traub, 2011; Sims et al., 2014), and again applies to a wide range of viruses (e.g., Walker et al., 2009; Meckes et al., 2010; Izquierdo-Useros et al., 2010; Meckes & Raab-Traub, 2011).

Moreover, the connection between PS and viruses is not limited to enveloped viruses, but extends to non-enveloped viruses (Clayson et al., 1989; Chen et al., 2015). In particular, see the Figure on the cover page of the Cell article by Chen et al., 2015, which shows “PS lipid vesicles” (essentially exosomes) and accompanies data showing that PS vesicles enable efficient en bloc transmission of enteroviruses. While not being bound by the particular mechanisms, the following rationale explains that PS is involved in infections from both enveloped and non-enveloped viruses.

All viruses orchestrate a timed exit of mature virions from the host cell to ensure successful infection of a new host cell. Enveloped viruses utilize the host cell plasma membrane to embed viral proteins that mediate efficient entry of the progeny virions with the next host cell. PS is found on the exterior of virus infected cells prior to virus release and enveloped viruses incorporate PS into the viral envelope upon exiting the host cell.

Viruses that do not incorporate an envelope into their mature virion leave the host cell by other mechanisms. Some strategies non-enveloped viruses use to release new virions from the cell include lysis of the cell, which can be caused directly by the host immune response to the infected cells (T cells or macrophages), or due to the activity of virus directly on host cell protein synthesis or cellular structures. An example of a virus alters the cell structure to induce cell lysis is Adenovirus. Adenovirus expresses several proteins late during infection that alter the structural integrity of the cell by disrupting filament networks and protein synthesis. Some non-enveloped viruses are able to release their progeny viruses via a nondestructive mechanism without any cytopathic effect. While poliovirus induces cell lysis rapidly (about 8 hours), it is also released from cells in PS lipid vesicles that are capable of infecting new host cells. Poliovirus particles in PS-vesicles are more efficient in infecting HeLa cells and primary macrophages than virus particles removed from PS-vesicles and blocking the vesicles with Annexin V inhibited the vesicles from infected cells in a dose dependent manner, suggesting the PS lipids are cofactors for poliovirus infection. In addition to poliovirus, Coxsackievirus B3 and Rhinovirus particles are also released into PS lipid vesicles (Chen et al., 2015), indicating a common mechanism utilized by enteroviruses to selectively release mature particles without lysis of the cell.

In regard to SV40, it is likely that SV40 is also released from cells in the above types of PS-lipid vesicles. For example, it has been reported that SV40 particles can be found released from cells before induction of cytopathic effects (Clayson et al., 1989). Also, SV40 virions have been observed in cytoplasmic smooth vesicles at 48 hour post infection and the release of SV40 particles was inhibited by monensin, a sodium ionophore that blocks intracellular protein transport by blocking cation transport across lipid membranes.

Also, many viruses need to induce activation of the host cell in order to create the environment in which to replicate efficiently. Cell activation by either viral or non-viral activating agents leads to rises in intracellular calcium (Ca₂₊) that activates PS translocation. Potential mechanisms of action of PS-targeting antibodies such as bavituximab thus include interference with proteins needed in cell activation or their ability to mediate viral egress, reversing the PS-mediated immunosuppression and clearance of infected cells or the virus by immune clearance mechanisms.

In vivo viral models demonstrate increased survival of virally-infected animals treated with PS-targeting antibodies. The potential mechanisms by which PS-targeting antibodies such as bavituximab have been shown to exert such anti-viral properties include: 1) binding to viral particles; 2) binding to infected cells; 3) inhibition of viral replication; and 4) enhancement of immune responses by blocking the immunosuppressive cell receptors that bind PS. Data in an HIV-1 model demonstrate that virions produced by virally infected macrophages have elevated levels of PS which serve as a cofactor for HIV-1 infection of macrophages. Blocking PS on HIV-1 with PS-targeting antibodies may prevent cell-cell interactions and block virus-target cell fusion. Results also indicate that bavituximab binds to pichinde viral particles and treatment of pichinde virus-infected guinea pigs enhances development of both of anti pichinde antibodies and cellular responses.

Overall, the treatment of all viral infections, including enveloped and non-enveloped viruses, using PS-targeting antibodies such as bavituximab is taught in U.S. Pat. No. 7,611,704 and U.S. Pat. No. 7,906,115, which supplement the present disclosure concerning such treatments. In particular, Table H, Table J and Table G of those patents exemplify the treatment of viral infections and associated diseases in animals and humans (Table H, Table J), along with common anti-viral drugs that may be used in combination therapies with PS-targeting antibodies such as bavituximab (Table G).

### K. Treating Cancer

Extensive sections of the present application concern treating tumors and cancer using PS-targeting antibodies such as bavituximab. The treatment of benign tumors is included, such as acoustic neuroma, neurofibroma, trachoma, pyogenic granulomas and BPH. The treatment of malignant tumors is preferred. As used herein, “tumor, tumors, cancer and cancers” are intended to indicate malignancy, unless expressly stated otherwise.

The treatment of blood-born tumors, such as leukemias and lymphomas, and various acute or chronic neoplastic diseases of the bone marrow is encompassed. Preferably, the tumors to be treated are solid or vascularized tumors, including tumors in which angiogenesis is active and tumors having prothrombotic blood vessels. “Solid” and “vascularized” tumors are tumors having a vascular component, i.e., which require tumor blood vessels for the provision of oxygen and nutrients to the tumor cells.

All cancers are included, whether primary or metastatic, as exemplified by breast, ovarian, thoracic, lung, liver (hepatocellular carcinoma, HCC), colon, colorectal, rectal, prostate, pancreatic, brain (gliomas and glioblastomas), cervical, uterine, endometrial, head and neck, parotid, esophageal, larynx, thyroid, gastrointestinal, stomach, kidney (renal cell carcinoma, RCC), biliary tract, bladder, testicular and other cancers, including carcinomas (squamous and non-squamous, small cell and non-small cell), adenocarcinomas and neuroblastomas, as well as melanoma, merkel cell carcinoma and hematological malignancies. In certain embodiments, the invention particularly applies to non-small cell lung cancer (NSCLC) or to breast, pancreatic, liver, kidney, rectal or ovarian cancer or melanoma. Most particularly, the invention applies to NSCLC such as non-squamous NSCLC. In addition to published literature, the treatment of all cancers using PS-targeting antibodies such as bavituximab is taught in a number of U.S. patents. For example, U.S. Pat. Nos. 6,406,693; 7,422,738; 8,486,391; 7,247,303; and 7,572,448, all supplement the present disclosure concerning such treatments. See also, the above discussion regarding therapeutically effective anti-cancer amounts (Section H2). As the modes of action of PS-targeting antibodies such as bavituximab are substantially or entirely the same in all solid tumors, it will be understood that the present invention is widely applicable to the treatment of all solid tumors, irrespective of the particular phenotype or genotype of the tumor cells themselves.

### L. Combination Therapies

Considerable sections of the present application, published literature and a number of U.S. patents also concern treating cancer using PS-targeting antibodies, such as bavituximab, in combination therapies (e.g., U.S. Pat. No. 7,422,738; U.S. Pat. No. 8,486,391; U.S. Pat. No. 7,572,448).

The biomarker and treatment methods may thus be combined with any other methods generally employed in the treatment of the particular disease or disorder that the animal or patient exhibits, particularly cancer and viral infections and diseases. So long as a given therapeutic approach is not known to be detrimental to the patient's condition in itself, and does not significantly counteract the PS-targeting antibody therapy, its combination with the present invention is contemplated. Combination therapies for non-malignant diseases are also contemplated.

In connection cancer treatment, the present invention may be used in combination with classical approaches, such as surgery, chemotherapy, radiotherapy, cytokine therapy, anti-angiogenesis and the like, and newer approaches such as immuno-oncology (IO) agents. The invention therefore provides biomarker and combined therapies in which the PS-targeting antibodies such as bavituximab are used simultaneously with, before, or after surgery or radiation treatment; or are administered to patients with, before, or after conventional chemotherapeutic or radiotherapeutic agents, cytokines, anti-angiogenic agents, apoptosis-inducing agents, targeted therapies, IO agents or such like.

In terms of surgery, any surgical intervention may be practiced in combination with the present invention. In connection with radiotherapy, any mechanism for inducing DNA damage locally within tumor cells is contemplated, such as γ-irradiation, X-rays, UV-irradiation, microwaves and even electronic emissions and the like. The directed delivery of radioisotopes to tumor cells is also contemplated, and this may be used in connection with a targeting antibody or other targeting means.

The general use of combinations of substances in cancer treatment is well known. When one or more agents are used in combination with PS-targeting antibodies such as bavituximab, there is no requirement for the combined results to be additive of the effects observed when each treatment is conducted separately. Although at least additive effects are generally desirable, any increased therapeutic effect or benefit (e.g., reduced side-effects) above one of the single therapies would be of value. Also, there is no particular requirement for the combined treatment to exhibit synergistic effects, although this is possible and advantageous.

The “primary therapeutic agents” or “first anti-cancer agents” for use with the present invention, as used herein, are the PS-targeting antibodies such as bavituximab. The “secondary or tertiary therapeutic agents” or “at least a second or third anti-cancer agent”, as used herein, are second or third, distinct therapeutic agents, anti-cancer or anti-viral agents, i.e., therapeutic agents, anti-cancer or anti-viral agents “other than” the primary therapeutic agent. Any secondary or tertiary therapeutic agent may be used in the combination therapies of the present invention. Also, secondary or tertiary therapeutic agents, “second or third anti-cancer agents” or “second or third anti-viral agents” may be selected with a view to achieving additive, greater than additive and potentially synergistic effects, according to the guidance in the present application and the knowledge of those of skill in the art.

To practice combined therapy, anti-tumor therapy or anti-viral therapy, one would simply administer to an animal or patient a PS-targeting antibody such as bavituximab in combination with another, i.e., a second or third, distinct therapeutic agent, anti-cancer or anti-viral agent, in a manner effective to result in their combined therapeutic, anti-tumor or anti-viral actions within the animal or patient. The agents would therefore be provided in amounts effective and for periods of time effective to result in their combined presence within the disease site, e.g., the tumor, tumor environment or microenvironment, and/or to exert their combined therapeutic actions in the animal or patient, preferably, to exert their combined therapeutic actions on the immune system of the animal or patient. To achieve this goal, the primary therapeutic agent and the second or third, distinct therapeutic agent may be administered substantially simultaneously, either in a single composition, or as two or three distinct compositions using different administration routes.

Alternatively, the PS-targeting antibody such as bavituximab may precede, or follow, the second or third, distinct therapeutic agent, anti-cancer or anti-viral agent by, e.g., intervals ranging from minutes to weeks or months. In certain embodiments where the primary therapeutic agent and the second or third, distinct therapeutic agent are applied separately to the animal or patient, one would ensure that an inoperative period of time did not exist between the time of each delivery, such that each agent would still be able to exert an advantageously combined effect. From standard practice, including the clinical experience to date with bavituximab, one or two weeks is not an inoperative period of time between administering bavituximab and a second or third, distinct therapeutic agent. Indeed, an interval of about one week may be preferred.

Moreover, one preferred combined tumor therapy concerns administering to an animal or patient a PS-targeting antibody such as bavituximab in combination with a second, distinct anti-cancer agent, either substantially simultaneously or preferably at intervals of weeks, followed by administering a third, distinct anti-cancer agent at a subsequent time and continuing the periodic administration of the third, distinct anti-cancer agent for a subsequently effective time, such as for a period of months.

The secondary or tertiary therapeutic agents for separately timed combination therapies may be selected based upon certain criteria, including those discussed herein and known in the art. However, a preference for selecting one or more second or third, distinct therapeutic agents for prior or subsequent administration does not preclude their use in substantially simultaneous administration if desired.

In terms of cancer, second or third, distinct anti-cancer agents selected for administration “prior to” the primary therapeutic agents, and designed to achieve increased and potentially synergistic effects, include agents that induce the expression of PS in the tumor microenvironment. For example, agents that stimulate localized calcium production, activate membrane transporters that move PS to the outer surface of the plasma membrane, injure the tumor endothelium, cause preapoptotic changes and/or induce apoptosis in the tumor endothelium or tumor cells will generally result in increased PS expression. Examples of such agents are docetaxel and paclitaxel. The PS can then be targeted using the PS-targeting antibody such as bavituximab, thus amplifying the overall therapeutic effect, and also giving increased attack via host effectors (complement, ADCC, antibody-mediated phagocytosis, CDC).

Drugs that have selectivity for angiogenic, remodeling or activated endothelial cells, such as are present in tumor blood vessels, but not in normal resting blood vessels, can also be used to selectively causes exposure of PS in the tumor microenvironment. Examples of such agents are combretastatins and docetaxel. This again would lead to increased antibody binding and enhanced initiation of host effector mechanisms.

Second or third, distinct anti-cancer agents selected for administration “subsequent to” the primary therapeutic agents, and designed to achieve increased and potentially synergistic effects, include agents that benefit from the effects of the primary therapeutic agent. PS-targeting antibodies such as bavituximab cause tumor necrosis. Accordingly, effective second or third, distinct anti-cancer agents for subsequent administration include anti-angiogenic agents, which inhibit metastasis; agents targeting necrotic tumor cells, such as antibodies specific for intracellular antigens that become accessible from malignant cells in vivo (U.S. Pat. Nos. 5,019,368; 5,882,626); and chemotherapeutic agents and anti-tumor cell immunoconjugates, which attack any tumor cells that may survive at the periphery. The currently most preferred second or third, distinct anti-cancer agents for administration subsequent to the PS-targeting antibody such as bavituximab are immune checkpoint inhibitors, as described below.

In some situations, it may be desirable to extend the time period for treatment significantly, where several days (2, 3, 4, 5, 6 or 7), several weeks (1, 2, 3, 4, 5, 6, 7 or 8) or even several months (1, 2, 3, 4, 5, 6, 7 or 8) lapse between the respective administrations. This would be advantageous in circumstances where one treatment was intended to substantially destroy the tumor, and another treatment was intended to prevent micrometastasis or tumor re-growth and/or to stimulate or assist in a host response against the tumor, such as the administration of an anti-angiogenic agent or an immune checkpoint inhibitor. Anti-angiogenics should be administered at a careful time after surgery, however, to allow effective wound healing. Anti-angiogenic agents may then be administered for the lifetime of the patient.

It is also envisioned that more than one administration of either the primary therapeutic agent or the second or third, distinct therapeutic agent will be utilized. The primary therapeutic agent and the second or third, distinct therapeutic may be administered interchangeably, on alternate days or weeks; or a sequence of one agent treatment may be given, followed by a sequence of the other treatment or treatments. In any event, to achieve a therapeutic effect using a combined therapy, all that is required is to deliver two or more agents in a combined amount effective to exert a therapeutic effect, irrespective of the times for administration.

L1. Chemotherapy

Whether administered substantially simultaneously or sequentially, the PS-targeting antibodies such as bavituximab may be administered in combination with one or more chemotherapeutic agents or drugs. Chemotherapeutic drugs can kill proliferating tumor cells, enhancing the necrotic areas created by the overall treatment. The drugs can thus enhance the action of the primary therapeutic agents of the invention.

Most cancer chemotherapeutic drugs are selective for dividing, oxygenated cells. These have advantages in combined therapy as the chemotherapeutic drug acts on different targets from the primary therapeutic agents, leading to a more complete anti-tumor effect. For example, chemotherapeutic drugs are selectively active against the rapidly dividing, oxygenated tumor cells in the tumor periphery. Anti-angiogenic drugs that are selective for well-oxygenated, angiogenic vessels in the tumor periphery would also be effective in combination.

By inducing the formation of thrombi in tumor vessels, the primary therapeutic agents of the present invention can also enhance the action of the chemotherapeutic drugs by retaining or trapping the drugs within the tumor. The chemotherapeutics are thus retained within the tumor, while the rest of the drug is cleared from the body. Tumor cells are thus exposed to a higher concentration of drug for a longer period of time. This entrapment of drug within the tumor makes it possible to reduce the dose of drug, making the treatment safer as well as more effective.

Further drugs for combined use in the present invention are those that act on cells that are “sensitized” to the drug by the action of the primary therapeutic agent, such that reduced doses of the second drug are needed to achieve its anti-tumor effect. For example, this could occur where a major component of the second drug's action is exerted on tumor blood vessels and the antibodies or agents of the invention sensitize the cells to the drug. The same is true where the primary therapeutic agent of the invention sensitizes tumor cells to a second drug, either directly or through stimulation of cytokine release.

Other suitable second or third anti-cancer agents for combination therapy are those that enhance the activity of host effector cells, e.g., by selectively inhibiting the activity of immunosuppressive components of the immune system. Such agents enable the primary therapeutic agents of the invention, which stimulate attack by effector cells as part of their mechanism, to work more aggressively. Examples of such gents are docetaxel and immune checkpoint inhibitors.

Although an understanding of the precise mechanism(s) of action of the primary therapeutic agents is not necessary to practice the treatment of the invention, data and reasoned deductions concerning such mechanisms can be used to select particular second or third anti-cancer agents for combined use in the present invention. The effectiveness of the chosen combination therapy, in turn, supports the original data and proposed mechanisms of action, and also leads to preferred categories of second or third anti-cancer agents for practicing combination therapy.

Drugs that induce apoptosis may be used in the combination therapies. Docetaxel, for example, induces apoptosis and therefore PS exposure by binding to microtubules and disrupting cell mitosis (Hotchkiss et al., 2002). Treatment of endothelial cells, which line tumor blood vessels, and tumor cells with docetaxel at subclinical concentrations is known to induce PS expression at the cell surface.

The anti-tumor effects of PS-targeting antibodies such as bavituximab include Fc domain-mediated augmentation of immune effector functions, such as ADCC, CDC, stimulation of cytokine production, and such mechanisms in combination. This is also relevant to docetaxel, as other studies have shown that the treatment of breast cancer patients with docetaxel leads to increases in serum IFNγ, IL-2, IL-6 and GM-CSF cytokine levels, augmenting the anti-tumor immune responses in these patients by enhancing the activity of natural killer (NK) and lymphokine activated killer (LAK) cells.

Therefore, docetaxel will both induce PS expression and binding of the administered antibody, and also enhance the activities of immune effectors, which mediate anti-tumor effects. Based upon the foregoing considerations, combination of the antibodies with docetaxel is a preferred embodiment, particularly when also combined with or followed by treatment with an immune checkpoint inhibitor, as described below.

Accordingly, docetaxel and other chemotherapeutic agents that induce apoptosis are certain preferred agents for use in the combination treatments of the present invention. Combinations with chemotherapeutics drugs that induce apoptosis, such as docetaxel, should synergistically attack tumor vasculature endothelial cell and tumor cell compartments, leading to not only significantly enhanced treatment efficacy but also lower toxicity. These combinations are contemplated for use in breast cancer treatment, particularly the combination of metronomic chemotherapy using docetaxel with a PS-targeting antibody.

Exemplary chemotherapeutic agents for combined therapy are described in U.S. Pat. No. 7,572,448 and U.S. Pat. No. 9,421,256 (e.g., as listed in Table D in U.S. Pat. No. 7,572,448 and in Table C in U.S. Pat. No. 9,421,256), such as, e.g., pemetrexed, temozolomide, tamoxifen, erlotinib, sunitinib, sorafenib, paclitaxel, carboplatin, gemcitabine and docetaxel. Other therapeutic antibodies may also be used, such as, e.g., trastuzumab, rituximab and bevacizumab. Each of those chemotherapeutic agents, antibodies and other drugs known in the art are exemplary and not limiting. Variation in dosage can occur depending on the condition treated. The treating physician will be able to determine the appropriate dose for the individual subject. In certain preferred embodiments docetaxel is used, such as docetaxel administered at a starting dose of 60 mg/m² or docetaxel administered to a patient in an amount of 75 mg/m².

### M. Immunotherapy (IO) Combinations

A challenge to effective immunotherapy is to overcome multiple pathways that inhibit innate or adaptive immune activation. The PD-1 immune checkpoint has been identified as a major immunosuppressive pathway and has emerged as a promising target for cancer immunotherapy with less toxicity than chemotherapy. It functions to exhaust activated tumor-specific T cells and dampen their tumor-killing activity. PD-1 is absent on naïve T cells, B cells, macrophages, DCs, and monocytes but expressed highly on their activated counterparts. Notably, tumors and associated myeloid cells exploit the PD-1 pathway to generate innate and adaptive immune resistance through up-regulation of PD-L1 expression. Mechanistic studies indicate that blockade of these immune checkpoints are most effective when there is a de novo or pre-existing anti-tumor immune response. Unfortunately, pre-existing tumor specific immune activity is limited in cancer patients because of the exposure of PS and other immunosuppressive factors that often dominate the tumor microenvironment.

Although durable anti-tumor immune responses have been observed in multiple cancer types with agents that block PD-1 signaling, only a subset of patients respond; consequently, a significant unmet medical need remains. In particular, patients that express low levels of PD-1 and PD-L 1 (a biomarker of immunosuppression and lack of T cell activation) in the tumor microenvironment appear less responsive to checkpoint blockade therapy. Observations of immune activation have shown that PS-targeting antibodies such as bavituximab may increase the proportion of patients who may benefit from anti PD-1/PD-L1 and other checkpoint therapies.

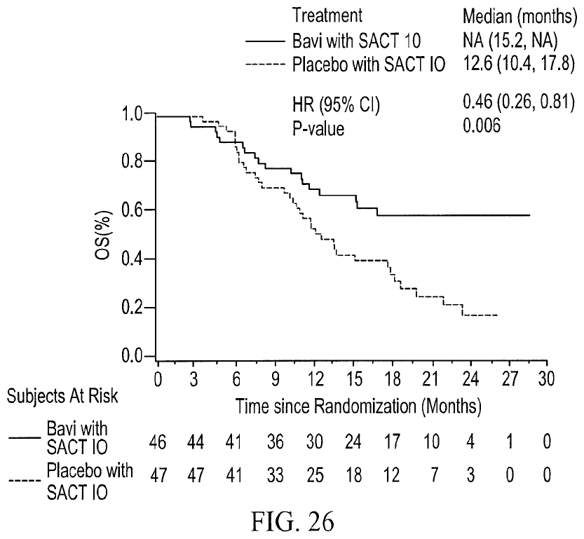

Presented herein are clinical data showing, for the first time, that human patients treated with bavituximab and immunotherapy have a meaningful survival advantage. In particular, the results in Example XIX demonstrate that patients treated with bavituximab (and docetaxel) followed by subsequent immunotherapy (“SACT-IO”) have a statistically significant better overall survival in comparison to patients treated with placebo (docetaxel alone) followed by subsequent immunotherapy. The prolonged survival was statistically significant (p=0.006) and even more impressive because mOS for bavituximab patients receiving subsequent IO has yet to be reached (Example XIX; FIG. 26; Table 16). Thus, bavituximab does, indeed, enhance the activity of immunotherapy agents in human patients.

Accordingly, as exemplified by the data in Example XIX, important embodiments of the present invention are the treatment of cancer patients with PS-targeting antibodies such as bavituximab in combination with immunotherapy or immuno-oncology (IO) agents. Exemplary immunotherapeutic agents for combined therapy are listed in Table C in provisional application Ser. No. 62/406,727, filed Oct. 11, 2016; and in Table D in each of provisional application Ser. No. 62/480,994, filed Apr. 3, 2017 and provisional application Ser. No. 62/507,580, filed May 17, 2017, of which combinations with NK cell and CAR-T therapies are currently preferred.

Certain preferred examples of IO agents are those approved for clinical treatment or in human clinical trials, preferably in late-stage clinical trials, such as those described in Table E in each of provisional application Ser. No. 62/480,994, filed Apr. 3, 2017 and provisional application Ser. No. 62/507,580, filed May 17, 2017. The doses for use and indications for treatment are well-known to those of ordinary skill in the art, as exemplified by the details in Table E in each of provisional application Ser. No. 62/480,994, filed Apr. 3, 2017 and provisional application Ser. No. 62/507,580, filed May 17, 2017. For example, nivolumab at 240 mg or 3 mg/kg every 2 weeks to treat melanoma, non-small cell lung cancer (NSCLC), renal cell carcinoma (RCC), classical Hodgkin (Hodgkin's) lymphoma, squamous cell carcinoma of the head and neck (head and neck cancer), bladder cancer, small cell lung cancer, brain cancers such as malignant glioma, anaplastic astrocytoma (AA) and glioblastoma multiforme (GBM), hepatocellular cancer (HCC), esophageal cancer, gastric cancer, mesothelioma and multiple myeloma; pembrolizumab at 200 mg or 2 mg/kg every 3 weeks to treat melanoma, NSCLC, classical Hodgkin lymphoma, head and neck cancer, gastric cancer, breast cancer, bladder cancer, all solid tumors, colorectal cancer, RCC, multiple myeloma, esophageal cancer and HCC; atezolizumab at 1200 mg every 3 weeks to treat bladder cancer, NSCLC, RCC, colorectal cancer, prostate cancer, melanoma, breast cancer, ovarian cancer and small cell lung cancer; avelumab at 10 mg/kg every 2 weeks to treat metastatic merkel cell carcinoma, NSCLC, ovarian cancer, gastric cancer, bladder cancer, RCC, diffuse large B-cell lymphoma (DLBCL), non-Hodgkin lymphoma and head and neck cancer; and durvalumab (MEDI4736) at 10 mg/kg every 2 weeks to treat NSCLC, head and neck cancer, bladder cancer and small cell lung cancer.

Other suitable IO agents are ipilimumab at 3 mg/kg or 10 mg/kg every 3 weeks to treat unresectable or metastatic melanoma; tremelimumab at 15 mg/kg every 3 months to treat melanoma; REGN2810 to treat NSCLC; PDR001 to treat melanoma; racotumomab to treat NSCLC; MEDI0562 and GSK3174998 to treat advanced solid tumors; urelumab to treat melanoma; utomilumab to treat DLBCL; and each of BMS-986016, LAG525, JNJ-61610588, TSR-022, MBG453, MEDI1873 and INCAGN01876 to treat solid tumors.

Particularly preferred IO agents for combination therapy with PS-targeting antibodies such as bavituximab, as directly supported by the data in Example XIX, are “checkpoint inhibitors”, also termed herein “immune checkpoint antibodies”. Suitable “immune checkpoint antibodies” include agonistic (activating) antibodies that bind to an activating immune checkpoint, receptor or molecule, such as CD28, OX40 and/or GITR, and antagonistic (blocking) antibodies that bind to an inhibitory immune checkpoint, receptor or molecule, such PD-1, PD-L1, CTLA-4, TIM-3 and/or LAG-3. Such blocking antibodies are routinely termed “immune checkpoint inhibitors”, which is also used herein. Several such antibodies are also described in Table E in each of provisional application Ser. No. 62/480,994, filed Apr. 3, 2017 and provisional application Ser. No. 62/507,580, filed May 17, 2017, as being approved for clinical treatment or in late-stage clinical trials.

The currently most preferred examples of immune checkpoint antibodies (immune checkpoint inhibitors) are “blocking antibodies that bind to CTLA-4, PD-1 or PD-L1”. Several such blocking antibodies that bind to CTLA-4, PD-1 or PD-L1, and methods, including functional assays, for their selection, preparation and use, are well-known to those of ordinary skill in the art, as described in Table B. These include blocking antibodies to CTLA-4, such as ipilimumab and tremelimumab; blocking antibodies to PD-1, such as nivolumab (Brahmer et al., 2015), REGN2810 and pembrolizumab (Garon et al., 2015); blocking antibodies to PD-L1, such as durvalumab (MEDI4736) and atezolizumab (Fehrenbacher et al., 2016); and combinations of any one or more of such antibodies, known as an “IO doublet”. Of these, tremelimumab, nivolumab, durvalumab and atezolizumab are currently preferred. The main U.S. patents for tremelimumab, nivolumab, durvalumab and atezolizumab are U.S. Pat. No. 6,682,736, U.S. Pat. No. 8,008,449, U.S. Pat. No. 8,779,108 and U.S. Pat. No. 8,217,149, respectively.

In addition to Table B, other suitable examples of anti-CTLA-4 antibodies are those described in U.S. Pat. No. 6,207,156, which particularly concerns anti-CTLA-4 antibodies that comprise a CDR (CDR3, CDR2 or CDR1) selected from a defined antibody from a deposited hybridoma.

In addition to Table B, other suitable examples of anti-PD-L1 antibodies are those described in U.S. Pat. No. 8,168,179, which particularly concerns treating PD-L1 over-expressing cancers with human anti-PD-L1 antibodies, including chemotherapy combinations; U.S. Pat. No. 9,402,899, which particularly concerns treating tumors with antibodies to PD-L1, including chimeric, humanized and human antibodies; and U.S. Pat. No. 9,439,962, which particularly concerns treating cancers with anti-PD-L1 antibodies and chemotherapy. These anti-PD-L1 antibody compositions and methods include those in development by Ono Pharmaceuticals and collaborators.

Further suitable antibodies to PD-L1 are those in U.S. Pat. No. 7,943,743, U.S. Pat. No. 9,580,505 and U.S. Pat. No. 9,580,507, kits thereof (U.S. Pat. No. 9,580,507) and nucleic acids encoding the antibodies (U.S. Pat. No. 8,383,796). Such antibodies bind to PD-L1 and compete for binding with a reference antibody; are defined by VH and VL genes; or are defined by heavy and light chain CDR3 (U.S. Pat. No. 7,943,743), or heavy chain CDR3 (U.S. Pat. No. 8,383,796), of defined sequences or conservative modifications thereof; or have 90% or 95% sequence identity to reference antibodies. These anti-PD-L1 antibodies also include those with defined quantitative (including binding affinity) and qualitative properties, immunoconjugates and bispecific antibodies. Further included are methods of using such antibodies, and those with defined quantitative (including binding affinity) and qualitative properties, including antibodies in single chain format and those that are in the format of an isolated CDR, in enhancing an immune response (U.S. Pat. No. 9,102,725). Enhancing an immune response, as in U.S. Pat. No. 9,102,725, may be used to treat cancer or an infectious disease, such as a pathogenic infection by a virus, bacterium, fungus or parasite. These anti-PD-L1 antibody compositions and methods include the product, BMS936559.

Further suitable antibodies to PD-L1 are those in U.S. Patent Application No. 2016/0009805, which concerns antibodies to particular epitopes on PD-L1, including antibodies of defined CDR sequences and competing antibodies; nucleic acids, vectors, host cells, immunoconjugates; detection, diagnostic, prognostic and biomarker methods; and treatment methods.

TABLE B
Blocking Antibodies to CTLA-4, PD-1 and PD-L1 and Functional Assays

| | Representative | Representative | U.S. Pat. | |
|---|---|---|---|---|
| Target | Generic Name | Brand Name | No. | Teaching |
| CTLA-4 | Ipilimumab | Yervoy ® | 6,984,720 | Antibodies to CTLA-4 defined by sequences; quantitative and |
| | | | | qualitative properties |
| | | | 7,605,238 | Antibodies to CTLA-4 defined by binding affinity, including |
| | | | | inhibitory and competitive binding assays; sequences |
| | | | 8,318,916 | Nucleic acids for antibodies to CTLA-4 defined by CDR3 sequences |
| | | | 8,784,815 | Increase immune responses and treat cancer with CTLA-4 antibodies |
| | | | 8,017,114 | defined by CDR3 sequences and affinity, or competition with |
| | | | | reference antibody and affinity |
| | Tremelimumab | | 6,682,736 | Antibodies to CTLA-4 (preferably human and primate) defined by |
| | | | | VH genes and aa substitutions therein; binding affinity, quantitative |
| | | | | properties, antibody competition |
| | | | 7,109,003 | Expressing antibodies to CTLA-4, defined by quantitative binding |
| | | | | properties or VL genes; affinity, antibody competition |
| | | | 7,132,281 | Host cells, expressing and purifying antibodies to CTLA-4, defined |
| | | | | by antibody competition (e.g., deposited antibody) and VL genes, |
| | | | | quantitative binding properties, 90%, 95% sequence identity |
| | | | 7,411,057 | Nucleic acids, host cells and expressing antibodies to CTLA-4, |
| | | | | defined by CDR sequences and deposited antibody |
| | | | 7,807,797 | Antibodies to CTLA-4, defined by heavy or light chain sequences |
| | | | 7,824,679 | (chain-shuffling) or CDRs, single chains and bispecifics, and |
| | | | 8,143,379 | treating cancer |
| | | | 8,491,895 | |
| | | | 8,883,984 | |
| PD-1 | Nivolumab | Opdivo ® | 8,008,449 | Antibodies to PD-1 that compete for binding with reference |
| | | | 8,779,105 | antibody; immunoconjugates; bispecifics; quantitative and |
| | | | 9,387,247 | qualitative properties |
| | | | 9,492,539 | Antibodies to PD-1 that compete for binding with reference |
| | | | 9,492,540 | antibody, 80%, 85%, 90%, 95%, 96%, 97%, 98% or 99%, |
| | | | | framework sequence identity, and treating cancer |
| | | | 8,728,474 | Treating tumors with antibodies to PD-1, including chimeric, |
| | | | | humanized and human |
| | | | 9,067,999 | Treating lung cancer with antibodies to PD-1 including |
| | | | | combinations |
| | | | 9,073,994 | Treating melanoma with antibodies to PD-1 including combinations |
| | | | 7,595,048 | Treating PD-L1 or PD-L2 over-expressing cancers with human anti- |
| | | | | PD-1 antibodies, including chemotherapy combinations |
| | Pembrolizumab | Keytruda ® | 8,354,509 | Competitive binding antibodies to PD-1; quantitative and qualitative |
| | | | | properties |
| | | | 8,900,587 | Antibodies to PD-1 defined by CDRs or certain sequences |
| | | | 8,952,136 | Antibodies to PD-1 defined by CDRs or certain sequences |
| PD-L1 | Durvalumab | [MEDI4736] | 8,779,108 | Antibodies to PD-L1 (B7-H1) that compete with deposited antibody |
| | | | | for binding to known epitope or have 90% identity to heavy and |
| | | | | light chain variable domains; competition assays for mutational |
| | | | | analyses |
| | | | 9,493,565 | Antibodies to PD-L1 (B7-H1) defined by CDRs or sequences |
| | Atezolizumab | Tecentriq ® | 8,217,149 | Heavy chain variable regions and antibodies that bind to PD-L1 |
| | | | | defined by CDRs with sequence variants, including effector-less Fc |
| | | | | mutations and aglycosylation; chemotherapy, anti-viral and vaccine |
| | | | | combinations |
| Combos | | | 9,084,776 | Treating tumors with antibodies to PD-1 and CTLA-4 |
| | | | [8,728,474] | |
| | | | [9,067,999] | |
| | | | [9,073,994] | |
| | | | 9,358,289 | Treating tumors with antibodies to PD-1 and CTLA-4, including |
| | | | | sub-therapeutic doses and PD-L1 negative tumors |
| | | | 9,393,301 | Treating tumors with antibodies to PD-L1 and CTLA-4 |
| | | | [9,402,899] | |

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example I

### Generation of the 3G4 Antibody

The present example describes the immunization protocol, the generation and initial characterization of the murine PS-targeting antibody termed 3G4.

To present anionic phospholipids, chiefly PS, to the immune system as stronger immunogens, they were formulated in a cellular context, most particularly as PS-positive cells. The membrane-exposed PS, surrounded by other membrane components, has a better conformation for raising antibodies. The intent was to immunize immunocompetent animals with autologous cells expressing PS, wherein the animals would not produce antibodies against all self, surface antigens, but would recognize the membrane-exposed PS as a foreign element.

Mouse endothelioma cells, bEnd.3 (immortalized mouse (BALB/c strain) endothelial cells) were cultured in 10% DMEM with 9 ml/500 ml HEPES Buffer, in 10% CO₂ incubator. The bEnd.3 cells were expanded in T175 TC flasks until the desired number of cells was obtained. Typically, each flask at ˜70-80% confluency has about 3×10⁶ cells, and each mouse should receive from 1×10⁶ to 20×10⁶ cells, up to 1×10⁷ cells.

bEnd.3 cells were treated with 50 μM to 200 μM of hydrogen peroxide for 1 or 2 hours at 37° C. to expose anionic phospholipids, particularly PS, before immunization. The stock of H₂O₂ was [9.8M]; 30% (v/v). This was diluted 1:1000, then 0.4 ml was added into the T175 TC flask with 40 ml media to a final concentration of 100 μM H₂O₂. The cells were maintained for 1 hour at 37° C. To harvest, the cells were washed 3× with warm phosphate-buffered saline (PBS), +10 mM EDTA, with a view to removing all BSA or serum protein in the medium. The cells were removed with gentle trypsin treatment, washed and centrifuged for 5 minutes at 1000 rpm. The supernatant was aspirated and the cells resuspended in DMEM without additives to the appropriate volume (each mouse received about 1×10⁷ cells in 200 μl) and kept on ice.

Cells treated in this manner were injected (200 μl of cell suspension) into BALB/c mice IP using 1 ml syringe and 23 gauge needle. Mice were first immunized from three to seven times at intervals of 3 to 4 weeks. Immune sera were collected by bleeding the mice ten days after each boost, starting from the second boost. The serum titer for PS antibodies was tested by ELISA.

These immunizations with autologous PS-positive cells did not result in unrestricted production of autoantibodies, but were limited to the production of antibodies reactive with PS, mainly antibodies reactive with PS in combination with other anionic phospholipids. Mice with extremely high titers of antibodies reactive with anionic phospholipids such as PS were obtained. The mice did not show any signs of toxicity.

In further immunizations, various mice were immunized three times with hydrogen peroxide-treated bEnd.3 cells and the serum was tested 54 days after the first immunization. IgG antibodies reactive with PS within serum were detected with an anti-mouse IgG, Fc specific secondary antibody (and IgM antibodies within serum were detected with an anti-mouse IgG mu specific secondary antibody). A number of effective antisera with IgG and IgM antibodies reactive with PS were obtained using this immunization protocol, of which the antisera with IgG antibodies were generally more effective.

Typically, when the IgG titer of the desired antisera for PS reached >200,000, but PC titer was <50,000, fusion was performed to generate the monoclonal antibody. Hybridomas were obtained by fusing splenocytes from immunized animals with myeloma partner P3X63AG8.653 cells (ATCC, Rockville, Md.).

An important aspect of this technique to prepare monoclonal antibodies useful in tumor treatment was the selection strategy, which involved screening to select antibodies that bind to anionic phospholipids, but not to neutral phospholipids. Another important aspect was to select antibodies that bind to PS-coated plates as strongly in the absence of serum as in the presence of serum. This was carried out with a view to excluding antibodies that recognize complexes of PS and serum proteins, such as complexes of PS and β2GPI.

The strategy to isolate monoclonal antibodies reactive with PS involved screening hybridoma supernatants on PS-coated plates using an anti-mouse IgG, Fc gamma specific secondary antibody. Screening was first conducted against four phospholipids (PS, phosphatidylserine; PE, phosphatidylethanolamine; CL, cardiolipin; and PC, phosphatidylcholine), as well as bEnd3 cells. Clones reactive with the neutral phospholipid, PC were discarded, as were clones non-reactive with bEnd3 cells. Clones with high binding to PS were selected. The wells that had PS only reactivity, or strong preference for PS were sub-cloned first, and wells that exhibited PS reactivity in combination with binding to other anionic phospholipids were sub-cloned second.

The isotype of each selected hybridoma was determined. As antibodies of IgG class have numerous advantages over IgM, including typically higher affinity, lower clearance rate in vivo and simplicity of purification, modification and handling, their generation was particularly desired. To focus on wells with homogeneous IgG isotype, wells containing IgM or a mixture of different Igs were discarded or re-cloned. Sub-cloning of highly positive clones was repeated three to four times.

An advantageous mouse IgG antibody (IgG3 κ) initially termed “F3-G4”, and re-designated as 3G4, was selected. The 3G4 antibody was tested for binding to PS in an ELISA in the presence and absence of serum, and was initially characterized as being “serum-independent”, i.e., an antibody that binds to PS in the absence of serum.

The 3G4 antibody was studied using the following “Standard ELISA”, which was used to test binding to PS or other phospholipids. The phospholipid antigen (PS antigen, P-6641 25 mg 10 mg/ml (solvent is Chloroform:MeOH 95:5) in 2.5 ml bottle) stock solution should be aliquoted and stored in an airtight container at −30° C. The preferred 96 well plates are Dynatech U bottom Immulon 1 (from Dynatech Labs, Cat #011-010-3550).

The standard blocking buffer used was 10% bovine serum dissolved in PBS. The primary antibody was the test sample. The secondary antibody was goat, anti-mouse IgG-HRP. The developing solutions were: 10 ml of 0.2M Na₂PO₄, 10 ml of 0.1M citric acid, one 10 mg tablet of OPD, and 10 μl of hydrogen peroxide. The stop solution was 0.18 M H₂SO₄.

The protocol entailed coating a 96-well plate with PS as follows: the PS stock solution was diluted in n-hexane to 10 μg/ml and mixed well. 50 μl was added to each well and allowed to evaporate for one hour. 200 μl of 10% serum (as a blocking buffer) was added to each well, covered and maintained at room temperature for 2 hours or overnight at 4° C. The plate was washed three times with PBS. The primary antibody (diluted in blocking buffer) was added and incubated for 2 hours at 37° C. The plate was washed three times with PBS. 100 μl well of secondary antibody (typically goat, anti-mouse IgG-HRP) was added and incubated for 1 hour at 37° C. The plate was washed three times with PBS. The ELISA was developed by adding 100 μl of developing solution to each of the wells, developed for 10 minutes; then 100 μl of stop solution was added to each plate and the O.D. read at 490 nm.

It was determined that the 3G4 antibody had a relative affinity for PS that was improved compared to prior antibodies, and that the 3G4 antibody bound to PS, CL, PI (phosphatidylinositol), PA (phosphatidic acid) and PG (phosphatidylglycerol). In keeping with the model for targeting PS differentially expressed in tumors, the 3G4 antibody did not react with the neutral phospholipids, PC and SM.

The 3G4 antibody was purified to apparent homogeneity from the supernatant of the cultured hybridoma using a standard Protein A procedure. Briefly, a sample containing the 3G4 antibody at physiological pH was applied to a Protein A column and allowed to slowly pass through, so that the IgG binds to immobilized Protein A. The column was washed with wash buffer to remove non-bound serum components. The 3G4 antibody was eluted from the column using an acidic elution buffer (about pH 2.8), and fractions containing the eluted antibody then neutralized or dialyzed to return to physiological pH. In testing this highly purified 3G4 antibody for binding to PS in an ELISA conducted in the absence of serum, it was still believed that 3G4 bound to PS directly, i.e., without serum or serum proteins.

### Example II

### Pre-Clinical Anti-Tumor Effects of the 3G4 Antibody

In this example, data are provided to exemplify early pre-clinical experience showing some of the anti-tumor effects of the 3G4 antibody in syngeneic and xenogeneic tumor models.

### A. Protocols for Animal Tumor Studies

The effects of 3G4 were first examined in syngeneic and xenogeneic tumor models. The general protocols for the animal tumor treatment studies are as follows.

The animals were obtained from Charles Rivers Laboratories. The mice were 4-5 weeks, female, C.B-17 SCID or Fox Chase SCID mice. Mice were housed in autoclaved caging, sterile food and water, with sterile handling. All procedures were performed in laminar flow hoods. Mice were acclimated 1 week and then ear-tagged and a blood sample (approximately 75-100 μl) taken from the tail vein to check for leakiness by ELISA. Any mice that failed the leakiness ELISA test were not used for test procedures. Mice were injected orthotopically with tumor cells into mammary fat pad (MFP) or subcutaneously into the right flank 2-3 days post ear-tagging and blood sample removal.

In the orthotopic model, 1×10⁷ cells in 0.1 ml DMEM were typically injected into MFP of anesthetized mice. Mice were anesthetized with 0.075 ml of mouse cocktail injected IP. The mouse cocktail is 5 ml Ketamine (100 mg/ml); 2.5 ml Xylazine (20 mg/ml); 1 ml Acepromazine (10 mg/ml); 11 ml sterile water. Dosage was 0.1 ml per 20-30 grams body weight via the IP route for a duration of 30 minutes.

Once the mouse was anesthetized, as measured by no response to toe/foot pinch, the mouse was laid on its left side and wiped with 70% ethanol just behind the head and around the right forearm/back area. A 2-3 mm incision was made just behind the right forearm (lateral thorax), which reveals a whitish fat pad when the skin flap is raised. 0.1 ml of cells were injected into the fat pad using a 1 ml syringe and a 27-gauge needle, producing a bleb in the fat pad. The incision was closed using a 9 mm sterile wound clip. The mouse was returned to its cage and observed until it had wakened from anesthesia and was mobile. Post-operative health status was determined, and if any signs of distress were observed, the animal was given acetaminophen (0.24 mg/ml)+codeine (0.024 mg/ml) in the drinking water. The wound clip was removed after 1 week. This method was used so that the cells are accurately placed into the selected site and not into the subcutaneous region. Tumors were approximately 200 μl in volume (L×W×W) in 14-15 days and the take rate was essentially 100%.

In the subcutaneous model, mice were typically injected with 1×10⁷ cells in 0.2 ml. Mice were not anesthetized, but were restrained using a steady grip of mouse skin exposing the right flank. A 1 ml syringe with a 23 gauge needle was used to inject 1×10⁷ cells in 200 μl, just under the skin of the mice and a bleb was seen. It was not unusual to observe a small amount of fluid leak from the injection site. A twisting motion was used when withdrawing the needle from the subcutaneous injection to reduce this leakage. Tumor volume was measured by L×W×H.

In the perfusion protocol, mice were injected IV with 1000 U of heparin in 0.2 ml saline. Mice were then sedated by injecting the mouse IP with 0.1 ml mouse cocktail. Once the mouse was sedated enough, as measured by no reflex when toe/foot is pinched, the thoracic cavity is opened to expose the heart and lungs. A 30 gauge needle attached to tubing and perfusion pump was inserted into the left ventricle. The right ventricle was snipped so that blood can drip out. Saline was pumped through for 12 minutes at a speed of 1 ml per minute. At the end of the perfusion, the needle and tubing were removed. Tissues were removed for further studies, either immunohistochemistry or pathology.

Differences in tumor growth rates were tested for statistical significance using a nonparametric test (Mann-Whitney rank sum test).

### B. Tumor Treatment Results

For the syngeneic model, Meth A mouse fibrosarcoma tumor cells were used with BALB/c mice. In xenogeneic models, human MDA-MB-231 breast tumor cells or MDA-MB-435 cells were seeded into the mammary fat pad of SCID mice. In another xenogeneic model, a large human Hodgkin's lymphoma L540 xenograft was established by injecting cells and allowing the tumor to grow to a size of over 500 mm³ before treatment. Tumor-bearing mice (8-10 animals per group) were injected i.p. with 100 μg of 3G4 antibody, purified to apparent homogeneity, as opposed to control antibody (termed BBG3, a mouse IgG3 κ antibody against a Babesia bovis antigen, secreted by a hybridoma obtained from the ATCC as 23.8.34.24; HB-10113). Treatment was repeated 3 times a week. Animals were monitored twice or thrice a week for tumor measurements.

The growth of both syngeneic and xenogeneic tumors was effectively inhibited by treatment with the 3G4 antibody (P<0.05). At the end of the studies, the average reduction in tumor growth in the 3G4-treated mice, as opposed to control mice, was 65% for MDA-MB-435 (FIG. 1A), 75% for MDA-MB-231 (FIG. 1B), 90% for Meth A (FIG. 1C) and 50% for L540 (FIG. 1D). No retardation of tumor growth occurred in control mice treated with the isotype-matched control antibody, BBG3. The treatment of the syngeneic, Meth A tumor cells was particularly successful. Even in mice bearing large L540 tumors, known to be resistant to necrosis, the 3G4 antibody treatment inhibited tumor growth in comparison to control. No toxicity was observed in mice treated with the 3G4 antibody.

In summary, the 3G4 antibody thus caused tumor vascular injury, localized thrombosis, tumor necrosis and retarded tumor growth, with no evidence of toxicity.

### Example III

### Generation of the Chimeric 3G4 Antibody, Bavituximab

The present example provides the full sequences of the heavy and light chain variable regions of the 3G4 antibody, which together include the six complementarity determining regions (CDRs), and describes the generation of chimeric versions of the 3G4 antibody, including the mouse-human chimeric antibody (ch3G4), now called bavituximab.

### A. 3G4 Antibody Sequences

The original sequences of the 3G4 antibody variable regions were obtained by RACE from the hybridoma that produces the 3G4 antibody and the sequences verified. The nucleic acid and amino acid sequences of the variable region of the heavy chain (Vh) of the 3G4 antibody are shown in FIG. 18A in U.S. Pat. No. 7,572,448. The heavy chain variable region sequence encompasses VH CDR1, VH CDR2 and VH CDR3, at locations predictable by Kabat (Kabat et al., 1991). The BstEII site in the nucleic acid sequence can be used as a convenient site to prepare a functional mouse variable region, e.g., for use in grafting onto a human constant region.

In practice, the 3G4-2BVH sequence has been grafted onto a human γ1 constant region at the BstEII site using a Lonza pEE vector. The resultant product contains the mouse leader sequence and its VH is joined to the human CH1 sequence in the manner shown in FIG. 18A in U.S. Pat. No. 7,572,448.

The nucleic acid and amino acid sequences of the variable region of the light chain (Vκ) of the 3G4 antibody are shown in FIG. 18B in U.S. Pat. No. 7,572,448. The light chain variable region sequence encompasses VL CDR1, VL CDR2 and VL CDR3, at locations predictable by Kabat (Kabat et al., 1991). The BbsI site in the nucleic acid sequence can be used as a convenient site to prepare a functional mouse variable region, e.g., for use in grafting onto a human constant region.

In practice, the 3G4-2BVL sequence has been grafted onto a human κ constant region at the BbsI site using a Lonza pEE vector. The resultant product contains the mouse leader sequence and its VL is joined within the human CL1 sequence in the manner shown in FIG. 18B in U.S. Pat. No. 7,572,448.

### B. Generation of the Mouse Chimeric Antibody, 2aG4

As described immediately below, the human chimera of the murine 3G4 antibody (ch3G4) is a human IgG₁ isotype (hIgG₁). The murine IgG homolog of ch3G4 corresponds to a mouse IgG₂ₐ isotype (mIgG₂ₐ). This construct was made and tested, and shown to behave essentially the same as the original mouse IgG₃ antibody.

Briefly, the 3G4 light chain coding sequence was amplified by RT-PCR from total RNA isolated from the 3G4 hybridoma cell line. RT-PCR primers were designed such that the amplified fragment contained XmaI and EcoRI restriction enzyme sites on either end of the amplified product for cloning into the Lonza expression vector, pEE12.4 vector. The variable region of the 3G4 heavy chain was amplified by RT-PCR from total RNA isolated from the 3G4 hybridoma cell line. Primers were designed such that the amplified fragment contained HindIII and XmaI restriction enzyme sites on either end of the amplified product for cloning into the Lonza expression vector, pEE6.4 vector.

The murine IgG2a constant region was amplified by PCR from a plasmid vector. PCR primers were designed with BstII and EcoRI restriction enzyme sites at either end of the amplification product for cloning into the pEE6.4+3G4VH vector. The BstEII site was designed to be in-frame with the 3G4 VH variable region sequence upstream. The heavy and light chain constructs were combined into a single double gene vector (12.4 3G4 IgG2a) by cutting both vectors with SalI and NotI. The heavy and light chain coding regions were verified by sequencing.

The 12.4 3G4 IgG2a vector was transfected into NS0 cells by electroporation. Following transfection, the NS0 cells were diluted and plated into 96-well plates in media lacking glutamine. Only cells transfected with the construct (which contains the glutamine synthease gene for positive selection) can grow in the absence of glutamine. Transfectants were identified and screened for antibody secretion using the Standard ELISA of Example I and those transfectants secreting the highest amounts of antibody were grown in large culture to generate purified antibody.

The resultant 2aG4 antibodies were purified to apparent homogeneity and shown to have essentially the same affinity and binding profile as the 3G4 antibody.

C. Generation of the Human Chimeric Antibody, ch3G4 (Bavituximab)

The chimeric construct containing the murine variable regions and the human constant regions has been produced (ch3G4) and shown to have essentially the same characteristics as the original murine antibody.

The murine 3G4 antibody was converted into a human-mouse chimeric antibody. The murine V_{H} was cloned and grafted onto the human γ₁ constant region at the BstEII site of the Lonza 2BVH vector. The murine V_{K} was cloned and grafted onto the human K constant region at the BbsI site of the Lonza 2BVL vector. The sequences were verified. The entire construct was expressed in CHO (Chinese hamster ovary) cells and the antibody purified. This is the antibody now called bavituximab.

The resultant ch3G4 bound at least as well as the murine 3G4 to phospholipid-coated ELISA plates using the Standard ELISA of Example I. The in vitro binding profile of chimeric 3G4 to the panel of phospholipids, PS, PA, CL, PI and PG, was shown to be the same as 3G4. The binding was antigen-specific, since no binding was observed with control antibodies of irrelevant specificity. In vivo, ch3G4 was also shown to localize to tumor vascular endothelium and to exert anti-tumor effects and anti-viral effects in a wide range of studies.

However, when the chimeric 3G4 construct was expressed in CHO cells under serum-free conditions, and the purified antibody tested for binding to PS in an ELISA in the absence of serum, binding to PS was lost.

### Example IV

### The 3G4 Antibody and Bavituximab Target PS in a β2GPI-Dependent Manner

This example provides data to resolve the apparent discrepancy in the PS binding profiles of the 3G4 antibody from the original hybridoma and the chimeric antibody expressed in CHO cells. In so doing, the present example demonstrates that the interaction between the 3G4 antibody and PS is dependent on the plasma protein, β2-glycoprotein I (β2GPI).

### A. Materials and Methods

### 1. Materials

Dulbecco's modified Eagle's medium (DMEM) and trypsin/EDTA were obtained from Mediatech, Inc. (Herndon, Va.). Fetal bovine serum (FBS), normal human serum, normal rat serum and normal mouse serum were obtained from Biomeda (Foster City, Calif.). Fresh human plasma was obtained from Carter Blood Care (Dallas, Tex.). Serum-free Hybridoma Media, Synthechol NS0 supplement, L-α-phosphatidylserine (PS), bovine serum albumin (BSA) and ovalbumin from chicken egg white (OVA) and were obtained from Sigma Chemical Co. (St. Louis, Mo.). DEAE cellulose, heparin-Sepharose and Hybond-P membranes were obtained from Amersham Biosciences (Buckinghamshire, UK). 1-palmitoyl-2-hydroxy-sn-glycero-3-phosphocholine [lysophosphatidylcholine (LPC)] was obtained from Avanti PolarLipids (Alabaster, Ala.). Ninety six-well Immulon-1B and -2HB microtiter plates were obtained from Thermo Lab Systems (Franklin, Mass.). Tris-HCl gradient SDS-PAGE gels and an Opti-4CN Substrate kit were obtained from Biorad (Hercules, Calif.). Eight-well glass chamber slides were obtained from BD Biosciences (Bedford, Mass.).

### 2. Antibodies

The 3G4 mouse monoclonal antibody, which was generated to bind the anionic phospholipid PS, is the antibody described in Example I. 3G4 was produced originally in hybridoma supernatant (Example I; Example II). 3G4 was also converted to a mouse IgG2a isotype (Example III, B) and was produced in the NS0 mouse myeloma cell line. NS0 cells were cultured in DMEM supplemented with 10% FBS or serum-free Hybridoma Media with Synthechol NS0 supplement. The human chimeric version of 3G4 (ch3G4; bavituximab) was generated (Example III, C) and the antibody produced from CHO cells under serum-free conditions.

The mouse anti-human β2GPI (anti-β2GPI or α-β2GPI) mAb was obtained from US Biological (Swampscott, Mass.). A hybridoma secreting C44, a colchicine-specific mouse IgG2a mAb, was obtained from the American Type Culture Collection (Rockville, Md.) and used as a control for 3G4 and anti-β2GPI. Rituximab (human IgG1 chimeric mAb) was used as a control for ch3G4. All secondary antibodies were obtained from Jackson Immunoresearch Labs (West Grove, Pa.).

### 3. Preparation of Antibody Fragments

3G4 F(ab')2 was generated by incubation with the protease pepsin. 3G4 Fab and control Fab 7H11 (anti-adenovirus) were generated by incubation with the protease papain. All antibody cleavage products were purified by FPLC, and verified by SDS-PAGE.

4. Purification of β2GPI from Human Plasma

Human β2GPI (hβ2GPI) for use in the present example was purified from human plasma essentially as described previously (Polz et al., 1980; Wurm et al., 1984). Briefly, perchloric acid (70%) was added to pooled plasma to a final concentration of 1.57% (v/v). The precipitate was discarded and the supernatant was adjusted to pH 7.5 with saturated Na₂CO₃, followed by extensive dialysis against 50 mM Tris, pH 8.0. This material was applied to a DEAE cellulose column equilibrated with 50 mM Tris, pH 8.0 to remove contaminants. The DEAE column flow-through was then applied to a heparin-Sepharose affinity column equilibrated with 50 mM Tris, pH 8.0, and bound proteins were eluted using 1.0 M NaCl. Finally, the β2GPI preparation was dialyzed against PBS and purified further by protein A/G to remove contaminating IgG. The final preparation contained a homogeneous band at 50 kDa, as shown by non-reduced SDS/PAGE and Coomassie® staining.

### 5. Construction and Expression of β2GPI and β2GPI Domains

To generate pure recombinant full-length and deleted forms ofβ2GPI, the yeast shuttle expression vector pPIC6αA (Invitrogen) and host strain Mut⁺X-33 (Invitrogen) were used. The expression vector contains the 5′ promoter and the 3′ transcription termination sequences of the alcohol (methanol) oxidase gene (AOX1). The vector also has a yeast α mating factor signal sequence downstream of the AOX1 promoter to which foreign cDNA can be fused for secretion of recombinant heterologous protein into the culture medium. Expression in P. pastoris provides glycosylation and disulfide bond formation similar to that in mammalian cells.

To generate the expression constructs, the following five expression constructs were made using human β2GPI cDNA, as described in Luster et al., 2006 and U.S. Pat. No. 8,956,616: the entire coding region of β2GPI cDNA without its cognate signal peptide (domain I-V); domain I deleted (domain II-V); domains I and II deleted (domain III-V); domains I, II and III deleted (domain IV-V); and domain V only (domains I, II, III and IV deleted). A common 3′ primer was used for PCR of all fragments.

PCR amplified fragments were inserted in-frame between the EcoR1 and XbaI restriction sites of pPICαA, directly downstream from the α mating factor signal sequence. A Stop codon was introduced at the end of each fragment to prevent fusion of the recombinant proteins to a c-myc epitope or a His tag at the C-terminus. Plasmid constructs were propagated in E. coli in presence of 100 μg/ml blasticidin and verified by restriction analysis and nucleotide sequencing. Recombinant proteins expressed by the above five constructs encoded proteins of approximately 36, 29, 24, 16 and 9 kDa, respectively, before glycosylation.

For the transformation and screening of expression clones, the recombinant plasmid constructs were linearized with restriction enzyme SacI, purified and 10 μg was used to transform host strain X-33 by the spheroplasts method (Invitrogen). Transformants for each of these constructs were selected on YPD (Yeast extract Peptone Dextrose Medium) plates containing 400 μg/ml blasticidin for 4 days. Several clones for each of these constructs were restreaked on YPD plates with 400 μg/ml blasticidin to determine the true integrants. Ten clones of each construct were then streaked on Minimal Dextrose (MD) and Minimal Methanol (MM) plates. Five clones of each construct, growing equally well on both MD and MM plates, were then grown in liquid MD and MM medium for 24, 48, 72, 96 and 120 hours. Supernatants and pellets for each clone at each time point were analyzed by Western blot using anti-human β2GPI polyclonal antibody. Clones that showed highest expression of the protein in supernatant were further used for large-scale preparation.

For the large scale purification of the recombinant proteins, recombinant proteins were produced using culture conditions recommended by Invitrogen. A starter culture of each clone was cultured in 5 ml of buffered minimal glycerol-complex medium (BMGY) at 30° C. with vigorous shaking overnight. Cells were collected, used to inoculate 25 ml of BMGY and grown for 2 days. Cells from the 25 ml culture were then used to inoculate 1 L of buffered minimal methanol-complex (BMMY) medium (1.0% methanol). Culture was continued for 4 days at 30° C. with vigorous shaking and 100% methanol was added every 24 hours (final concentration of 1.0%) to maintain protein expression. Culture medium was clarified by centrifugation (4000 ×g, 15 min) and supernatant was dialyzed for 2 days at 4° C. in 50 mM Tris buffer before being applied to a DEAE-sephacel column equilibrated with 50 mM Tris buffer. Flow through solution was collected and applied to a heparin-sepharose column. β2GPI was eluted from heparin-sepharose column with 1 M NaCl, dialyzed against 50 mM Tris buffer, concentrated using Amicon concentrator and analyzed by Western blot. The N-terminus of each protein was sequenced to confirm cleavage of the a-factor leader sequence. Protein yields varied from 10 mg/L (full-length β2GPI) to 25 mg/L (β2GPI domain V).

6. Preparation of “Nicked” hβ2GPI

Nicked hβ2GPI was prepared from intact hβ2GPI purified from human plasma as described above in the present example. hβ2GPI was incubated with plasmin-coated beads at 37° C. for 17 hrs. The beads were removed by centrifugation and the supernatant containing the cleaved protein was recovered. Western blotting of the purified product indicated the nicked β2GPI preparations were plasmin-free and did not contain plasmin autoproteolytic products (no reactivity with anti-plasmin or anti-angiostatin antibodies). N-terminal sequence analysis revealed two N-termini that corresponded to the N-terminus of β2GPI and a new sequence generated at the Lys317/Thr318 cleavage site.

### 7. PS ELISAs

The Standard ELISA of Example I was adapted with the following modifications. PS-coated Immunlon 1B microtiter plates were blocked overnight in 1% OVA (w/v). The following day, serial 2-fold dilutions of 3G4 purified from serum-containing or serum-free supernatant were prepared from an initial concentration of 13.33 nM. Dilutions were performed in 1% OVA or 10% non-heat inactivated sera from cow, human, rat or mouse. Plates were incubated for 1 hr. at 37° C. and binding of 3G4 was detected. All ELISA studies were performed at least three times.

### 8. Anti-hβ2GPI ELISA

The assay was performed as described above with the following modifications. hβ2GPI, nicked hβ2GPI, or recombinant hβ2GPI peptides were coated on 96-well Immunlon 2HB microtiter plates overnight at a concentration of 10 μg/ml. Plates were then blocked in 1% OVA for 1 hr. at room temperature. 3G4, ch3G4, or anti-β2GPI were diluted in 1% OVA to an initial concentration of 13.33 nM and serial 2-fold dilutions were prepared. Plates were incubated for 1 hr. at 37° C. and antibody binding was detected. All ELISA studies were performed at least three times.

### 9. Western Blot

Protein samples were heated to 95° C. for 5 min in non-reducing SDS sample buffer. The samples were then loaded onto a Tris-HCl 4-15% gradient SDS-PAGE gel and separated using a Mini Protean II apparatus (Biorad). Separated proteins were transferred to a PVDF membrane and blocked overnight in 3% BSA (w/v). Membranes were probed with anti-β2GPI, 3G4, or control mouse IgG diluted to 1 μg/ml in 3% BSA, washed thoroughly and incubated with peroxidase-labeled goat anti-mouse IgG. Membranes were developed using an Opti-4CN Substrate kit.

### 10. Induction and Detection of PS Exposure on Endothelial Cells

Adult bovine aortic endothelial (ABAE) cells were maintained in DMEM supplemented with 10% FBS and 2 mM L-glutamine. ABAE cells were removed from subconfluent cultures by brief exposure to 0.25% trypsin/0.02% EDTA and 8-well chamber slides were seeded with 2×10⁴ cells/well. Following overnight culture, cells were washed gently with PBS and treated with 200 μM lysophosphatidylcholine (LPC) to induce PS exposure. LPC-treatment was performed in the presence of 3G4, ch3G4, or control IgG for 30 min at 37° C. in either 10% FBS or 10% normal mouse serum (MS). If LPC-treatment was performed in 10% MS, hβ2GPI was added as a co-factor because 3G4/ch3G4 cannot bind PS in MS (see Results below in the present example).

PS exposure was determined by immunofluorescence staining. Cells were washed thoroughly in PBS, fixed in 4% paraformaldehyde (w/v) and incubated with a biotin-conjugated anti-mouse secondary antibody. Next, cells were incubated with FITC-conjugated streptavidin (Jackson Immunoresearch) to detect antibody binding. Cells were then permeabilized with 0.1% Triton-X100 in PBS and counterstained with Texas Red-conjugated phalloidin (Molecular Probes, Eugene, Oreg.) and 4′, 6-diamidino-2-phenylindole (DAPI; Molecular Probes). Images were captured using a Coolsnap digital camera (Photometrics, Tucson, Ariz.) mounted on a Nikon microscope and processed with MetaVue software (Universal Imaging Corporation, Downingtown, Pa.).

### 11. Quantification of Antibody Binding to ABAE Cells

The area of antibody binding was determined using MetaVue image analysis software, which is able to quantify the number of illuminated pixels in an image. Images of FITC fluorescence were used to quantify antibody binding. Corresponding images of DAPI fluorescence were used to normalize the FITC images for the number of cells present in the field. A small FITC/DAPI ratio indicates a small antibody binding area, whereas a large FITC/DAPI ratio indicates a large binding area. The FITC/DAPI ratios were used to determine increases or decreases in antibody binding area relative to a basal amount of antibody binding under the selected conditions. Five images at 200x magnification were used for each analysis. Data are analyzed as average relative FITC/DAPI ratios with standard deviation.

### B. Results

Overall, the following data demonstrate that the interaction between the 3G4 (and bavituximab) antibodies and PS is dependent on the plasma protein, β2GPI. 3G4 is shown to bind to β2GPI at domain II, which is not linked to pathogenic antibodies isolated from patients with Anti-Phospholipid Syndrome (APS), which commonly recognize β2GPI domain I. The data show that divalent 3G4/β2GPI complexes are required for enhanced PS binding, including to PS-positive cells, since 3G4 Fab′ fragments do not have this activity.

### 1. 3G4 Requires a Serum Factor to Bind PS-Coated Microtiter Plates

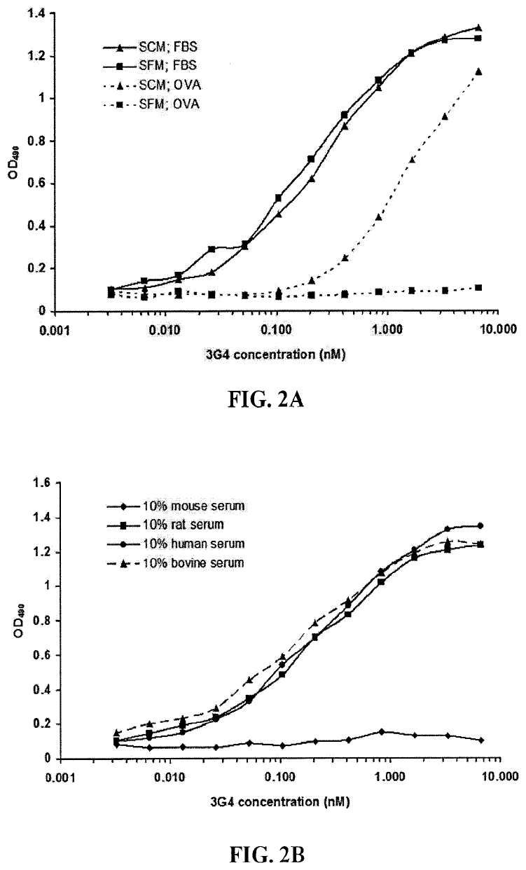

The 3G4 antibody purified from serum-containing media (SCM) or serum-free media (SFM) binds to PS-coated microtiter plates when serial dilutions are performed in 10% FBS (FIG. 2A, solid lines). In contrast, when serial dilutions are performed in 1% OVA (which lacks bovine serum proteins), 3G4 purified from SFM no longer binds PS (FIG. 2A, dashed line, ▪). This finding indicates that a factor present in bovine serum mediates the interaction between 3G4 and PS.

Interestingly, 3G4 purified from SCM still binds to PS when serial dilutions are performed in 1% OVA (FIG. 2A, dashed line, ▴). Clearly, serum proteins from the serum-containing media are mediating the interaction between 3G4 and PS. This indicates that even though 3G4 grown in SCM was purified, the low levels of serum proteins that can exist in the purified antibody are still sufficient to support PS binding.

In light of these findings, the studies described below were performed using 3G4 purified from SFM.

2. 3G4 Binding to PS in Sera from Different Species

To determine whether sera from other mammalian species can mediate the interaction between the 3G4 antibody and PS, serial dilutions of 3G4 were performed in 10% mouse, rat, human or other sera. The 3G4 antibody bound PS in the presence of rat and human serum, much like in the presence of bovine serum (FIG. 2B). However, 3G4 did not bind PS in the presence of mouse serum (FIG. 2B). In related studies, 3G4 bound PS in the presence of hamster, ferret, guinea pig, rabbit and monkey serum. Therefore, the serum protein epitope recognized by 3G4 is conserved among all mammalian species tested except mouse.

### 3. 3G4 Binds the Serum Glycoprotein, β2GPI

In the early 1990s, it was shown that many so-called anti-phospholipid (aPL) antibodies do not recognize phospholipids directly, but instead bind to serum proteins that, in turn, have affinity for phospholipids (Galli et al., 1990; McNeil et al., 1990). Therefore, a panel of human serum proteins known to interact with anionic phospholipids was screened for reactivity with the 3G4 antibody.

For example, human β2GPI (hβ2GPI) was coated on a microtiter plate and incubated with mouse anti-human β2GPI antibody (anti-β2GPI), the 3G4 antibody, or a control mouse IgG2a of irrelevant specificity (control mIgG). As expected, anti-β2GPI bound to hβ2GPI, while the control mIgG did not (FIG. 3). The 3G4 antibody also bound strongly to the hβ2GPI coated plate (FIG. 3).

To determine whether β2GPI is the only serum protein recognized by the 3G4 antibody, purified hβ2GPI and 10% human serum were run on an SDS-PAGE gel and transferred to a membrane support for immunoblot. 3G4 detected the 50 kDa purified hβ2GPI and a single band of similar size in human serum. Importantly, the 3G4 immunoblot was virtually identical to a blot generated using the anti-β2GPI antibody. The control mIgG antibody did not detect any protein.

Other human serum proteins known to interact with anionic phospholipids were tested to confirm lack of reactivity with the 3G4 antibody in ELISAs. Equal amounts of the particular protein were coated on microtiter plates, blocked in 1% OVA and incubated with a serial dilution of the 3G4 antibody. Plates were washed thoroughly and antibody binding was detected with a peroxidase-labeled secondary detection antibody. All studies included positive and negative control antibodies, which performed as expected. The results of the immunoblot and ELISA studies are: positive, β2GPI; negative, annexin V, Factor XII, kininogen (low- or high-molecular weight), oxidized LDL, Protein C, Protein S, prothrombin and tissue plasminogen activator (tPA). Together, these data indicate that the 3G4 antibody binds the serum protein, β2GPI.

### 4. 3G4 Binds β2GPI at Domain II

β2GPI has five domains, of which the fifth domain is responsible for binding to anionic phospholipids such as PS. To determine which domain of β2GPI is recognized by the 3G4 antibody, recombinant human β2GPI constructs were generated with different domain structures and tested alongside recombinant full-length hβ2GPI. These domain constructs were made by serial truncations from the N-terminus, and so lack each of the N-terminal domains in turn, as follows: recombinant full-length hβ2GPI contains domains I-V; hβ2GPI from which domain I has been deleted contains domains II-V; hβ2GPI from which domains I and II have been deleted contains domains III-V; hβ2GPI from which domains I, II and III have been deleted contains domains IV-V; and hβ2GPI from which domains I, II, III and IV have been deleted contains domain V only.

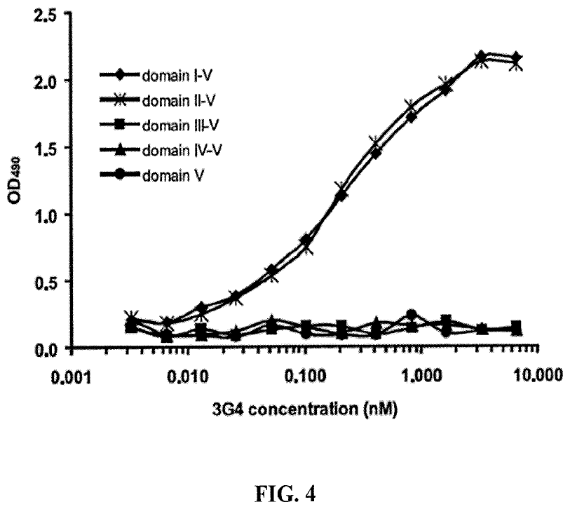

Equal amounts of the full-length hβ2GPI and each of the above hβ2GPI domain constructs were coated on microtiter plates and incubated with a serial dilution of the 3G4 antibody. This study showed that only hβ2GPI constructs containing domain II of β2GPI (domains I-V and domains II-V) were detected by 3G4 (FIG. 4). When domain I was deleted, 3G4 bound equally well to domains II-V (FIG. 4). Thus, the 3G4 antibody binds to β2GPI at domain II.

The finding that the 3G4 antibody binds to β2GPI at domain II is important in light of the information known about pathogenic antibodies isolated from patients with APS. Pathogenic anti-β2GPI antibodies isolated from patients with APS commonly recognize domain I of β2GPI (de Laat et al., 2005). Anti-β2GPI antibodies from APS patients that recognize domain II are not often pathogenic. This likely explains the lack of toxicity associated with 3G4 following toxicological studies performed in a variety of animal models, and in extensive clinical experience, such as described herein.

5. Co-Binding of 3G4 and β2GPI to Cells with Exposed PS

To verify the above findings under more physiological conditions, a live-cell binding assay was developed. This assay detects and measures antibody binding to cell membrane surfaces following treatment with the membrane disrupting agent, lysophosphatidylcholine (LPC) to induce PS exposure.

In this assay, ABAE cells were incubated with the 3G4 antibody or control mIgG in DMEM +10% FBS in the presence or absence of 200 μM LPC for 30 min. Cells were then washed, fixed and stained with fluorescent markers to visualize binding of antibody to the cell surface. The pixel area of 3G4 or mIgG binding was quantified using MetaVue software. All values were relative to the binding of 3G4 to non-LPC treated cells, which was set to one.

When the 3G4 antibody was added to ABAE cell culture media under normal conditions, no binding to the cells was observed. However, when ABAE cells are incubated with 3G4 in the presence of LPC, numerous pinpoints of 3G4 antibody binding are readily detectable. LPC is known to induce temporary membrane distortions (Kogure et al., 2003), which likely cause a loss of membrane asymmetry and exposure of PS.

Quantification showed that the area of 3G4 antibody binding increased greater than 500-fold upon LPC-treatment, while binding of a control mIgG remained undetectable. Similar results were obtained previously, when 3G4 and the PS-binding molecule annexin V were shown to bind endothelial cells following induction of PS exposure with H₂O₂ (Ran et al., 2005; U.S. Pat. No. 8,486,391). LPC-treated ABAE cells were not stained by the membrane impermeant dyes propidium iodide or DAPI, indicating 3G4 bound PS on the cell surface, not the inner leaflet of the plasma membrane.

To confirm that β2GPI is required for binding of the 3G4 antibody to cells with exposed PS, the live-cell binding assay was performed in media containing 10% mouse serum rather than 10% FBS to prevent interference from bovine β2GPI. As demonstrated above, the 3G4 antibody does not bind PS in the presence of mouse serum. Furthermore, the 3G4 antibody did not detect any protein in 10% mouse serum by immunoblot, indicating that 3G4 does not recognize murine β2GPI.

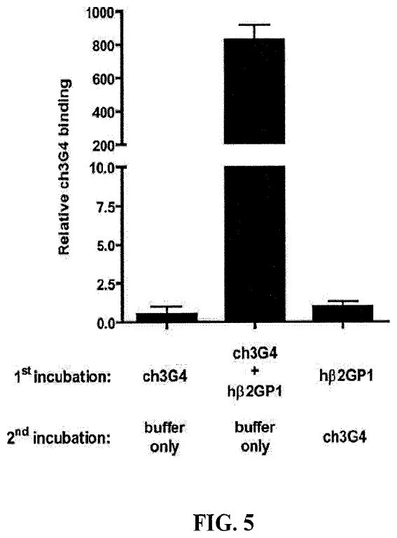

For this study, the human chimeric 3G4 antibody (ch3G4) was used to prevent background caused by detection of murine IgG present in mouse serum. When ABAE cells were incubated with ch3G4 in the presence of 10% mouse serum and LPC, no antibody binding was detected (FIG. 5). In contrast, addition of purified hβ2GPI to the binding reaction supported widespread binding of ch3G4 (FIG. 5), demonstrating that the ch3G4 antibody binding is dependent upon hβ2GPI. In all situations, ch3G4 binding was dependent upon LPC treatment and no binding was detected using a control human IgG of irrelevant specificity.

Interestingly, when ABAE cells were incubated with hβ2GPI in the presence of 10% mouse serum and LPC, washed thoroughly, then incubated with the ch3G4 antibody to detect binding of hβ2GPI, very little ch3G4 binding was detected (FIG. 5). This finding indicates that hβ2GPI does not bind cells with exposed PS in the absence of the ch3G4 antibody, and is consistent with reports that β2GPI has a low affinity for PS membrane surfaces under physiological conditions (Willems et al., 1996; Bevers et al., 2004; Bevers et al., 2005). Together, these data show that the ch3G4 antibody and hβ2GPI must be present simultaneously to bind ABAE cells with exposed PS, suggesting the ch3G4 antibody enhances the affinity of β2GPI for PS.

### 6. The Lipid Binding Region of β2GPI is Required for Co-Binding of 3G4

To confirm that the lipid binding region of β2GPI is required for co-binding of β2GPI and the 3G4 and ch3G4 antibodies to cells with exposed PS, the live-cell binding assay was performed using “nicked” hβ2GPI. Nicked hβ2GPI is unable to bind PS due to plasmin-mediated cleavage within the lipid binding region of domain V (Hunt et al., 1993; Hunt & Krilis, 1994).

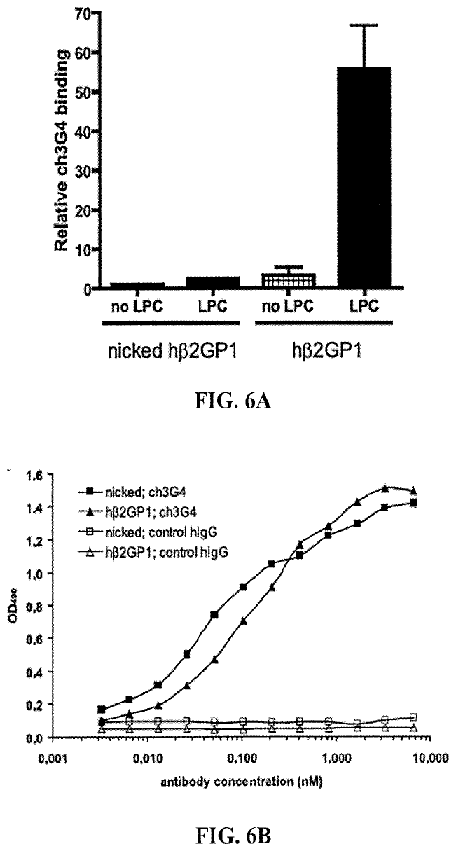

When ABAE cells are incubated with the ch3G4 antibody and hβ2GPI or nicked hβ2GPI in the absence of LPC, no ch3G4 binding is detected (FIG. 6A). In the presence of LPC, hβ2GPI is able to mediate binding of ch3G4 to ABAE cells with exposed PS, while nicked hβ2GPI is not able to mediate binding (FIG. 6A). The lack of binding in the live-cell assay is not due to an inability of the ch3G4 antibody to bind nicked hβ2GPI, since ch3G4 binds nicked hβ2GPI as well as hβ2GPI when equal amounts of protein are coated on microtiter plates (FIG. 6B). These findings demonstrate that the ch3G4/hβ2GPI complex detects PS exposed on ABAE cells through the lipid binding region of hβ2GPI domain V.

### 7. Antibody Divalency is Required for Co-Binding of β2GPI

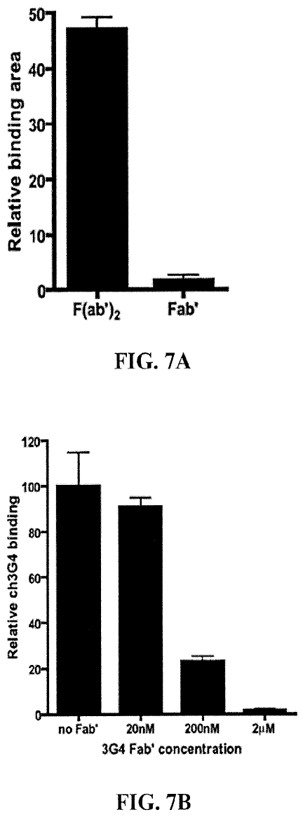

The data presented above suggest that the 3G4 antibody detects PS by enhancing the avidity of β2GPI for anionic phospholipids. To determine whether divalency is required for 3G4/β2GPI binding to cells with exposed PS, 3G4 F(ab′)₂ and 3G4 Fab′ monomers were generated and used in live-cell binding assays with the intact 3G4 antibody. As expected, the intact 3G4 antibody bound to LPC-treated ABAE cells, but not to untreated cells. An equivalent concentration of 3G4 F(ab′)₂ also bound to LPC-treated ABAE cells (FIG. 7A), but binding of 3G4 Fab′ was negligible (FIG. 7A). An apparent decrease in binding of 3G4 F(ab′)₂ relative to 3G4 was detected and is likely due to lost binding of the polyclonal secondary antibody to Fc epitopes missing on 3G4 F(ab′)₂. No binding of 3G4 Fab′ was detectable on ABAE cells even at a concentration of 2 μM, which is 1,000-fold above the concentration required to bind β2GPI coated on microtiter plates.

Moreover, 3G4 Fab′ inhibited ch3G4/β2GPI binding to LPC-treated ABAE cells in a concentration-dependent manner (FIG. 7B), while a control Fab′ of irrelevant specificity did not. The ability of 3G4 Fab′ to inhibit ch3G4 binding confirms that 3G4 Fab′ is able to bind β2GPI and that monomeric 3G4 Fab′/β2GPI complexes cannot bind cells with exposed PS. These data show that divalent 3G4/β2GPI complexes are required to bind to PS exposed on cell surfaces.

In summary, as shown in FIG. 4, the 3G4 antibody binds to β2GPI at domain II, and as shown in FIG. 6A and FIG. 6B, the lipid binding region of β2GPI domain V is required for co-binding of 3G4 (and ch3G4) and β2GPI to PS exposed on cells. In addition, as demonstrated in FIG. 7A and FIG. 7B, antibody divalency is required for such co-binding of 3G4 (and ch3G4) and β2GPI to exposed PS. Accordingly, there is presented a model of antibody and β2GPI co-binding to PS exposed on the outer surfaces of membranes, such as occurs on activated endothelial cells, tumor vascular endothelial cells and tumor cells, as well as on virally infected cells (FIG. 8).

### Example V

### Pre-Clinical Modelling of the Interactions Between Bavituximab and β2GPI

The present example provides pre-clinical data concerning interactions between the bavituximab family of antibodies, β2GPI and PS. Overall, the data show that relatively low levels of β2GPI, markedly below the typical amounts in the human population, are sufficient for effective binding of bavituximab to PS.

### A. Low β2GPI Supports Anti-Tumor Effects in Mice

In the initial development of the murine 3G4 antibody, the antibody was purified to apparent homogeneity from the supernatant of the cultured hybridoma using a standard Protein A procedure (Example I). In early studies in mice, this purified antibody was shown to exert anti-tumor effects in several models (Example II).

After it was determined that the 3G4 antibody requires β2GPI for PS binding (Example IV), and that all species of β2GPI support PS binding except mouse (FIG. 2B), it was deduced that the anti-tumor effects of Example II are under-estimates of the potency of the 3G4 antibody. That is, the anti-tumor effects of the 3G4 antibody in FIG. 1A, FIG. 1B, FIG. 1C and FIG. 1D are supported by only the low level of bovine β2GPI that could have co-purified with the 3G4 antibody through the Protein A column. Basically, although the majority of the protein delivered was pure 3G4 antibody, low levels of bovine β2GPI must have been co-administered to the mice (originating from the 10% fetal bovine serum (FBS) used to culture the hybridoma cells). These initial data therefore suggest that high levels of β2GPI are not necessary for the 3G4 antibody to inhibit tumor growth in vivo.

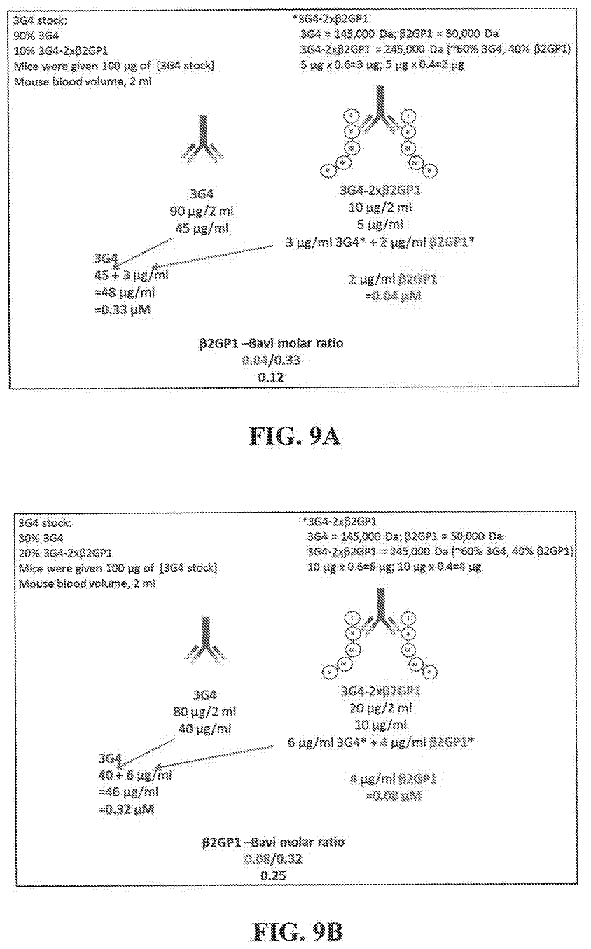

The 3G4 antibody was purified to apparent homogeneity using a Protein A column, which purifies antibodies based on the affinity for IgG. There are further reasons why β2GPI from the culture media would have separated from the 3G4 antibody during purification. Firstly, the affinity of β2GPI for the 3G4 antibody is low (and less than the affinity between Protein A and 3G4), such that β2GPI would have separated from 3G4 during loading and washing. Secondly, the low pH elution step (to separate the 3G4 antibody from Protein A) would have removed β2GPI complexed to 3G4. Also, on collecting eluted protein in the main antibody peak, the smaller β2GPI protein would not have been collected. Nonetheless, as shown in FIG. 9A and FIG. 9B, even taking the hypothetical position that 10-20% of the apparently pure 3G4 antibody delivered to the mice was actually in form of 3G4-β2GPI complexes, such levels of bovine β2GPI are still very low in absolute terms and in comparison to the antibody.

In more detail, the 3G4 antibody was produced from hybridoma cells in 10% FBS. A one liter volume would typically yield 10 mg/L of 3G4 antibody. Assuming that FBS contains 200 μg/ml of bovine β2GPI (similar to the level of human β2GPI in human sera), 10% FBS would contain 20 μg/ml of bovine β2GPI. At collection, the one liter hybridoma cell supernatant would contain 10 mg of the 3G4 antibody and a maximum of 20 mg of bovine β2GPI (20 μg/ml×1,000=20 mg). Upon purification with Protein A, the resulting material would contain 10 mg of protein. Assuming a 3G4 purity of 80-90%, the remaining 10-20% is a 3G4:β2GPI “complex”, in which one antibody is attached to two β2GPI (3G4-2xβ2GPI).

Mice were administered 100 μg of such protein. At 90% purity, 90 μg of the 100 μg of administered protein is pure 3G4, with 10 μg of 3G4-2xβ2GPI complex. Taking the molecular weight (MW) of the antibody to be 145 kD, and the MW of β2GPI to be 50 kD (Example IV, A4,B3; McNeil et al., 1990; Luster et al., 2006), and with two β2GPI for each antibody in the complex, the ratio by weight is approximately 3:2 (precisely, 59.2% antibody and 40.8% antibody). Therefore, of the 10 μg of complex, approximately 6 μg is 3G4, and 4 μg is β2GPI (precisely, 5.92 μg 3G4, and 4.08 μg is β2GPI).

The volume of blood of a mouse is 2 ml. The pure 3G4 antibody is present at 90 μg per mouse, or 45 μg/ml. At the ratio of about 3:2, 10 μg of complex per mouse contains about 6 μg of 3G4 and about 4 μg of β2GPI (3 μg/ml of 3G4 and 2 μg/ml of β2GPI). At 2 μg/ml of β2GPI capable of antibody binding, this is about 1% of the average β2GPI levels in human sera, and yet supported anti-tumor activity (FIG. 1A, FIG. 1B, FIG. 1C and FIG. 1D).

The 2 μg/ml of β2GPI in the mouse corresponds to 0.04 μM. As to 3G4, adding the 3 μg/ml of 3G4 from the administered 3G4-2xβ2GPI complex to the 45 μg/ml pure 3G4, there is 48 μg/ml of the 3G4 antibody. This corresponds to antibody at 0.33 μM. At a starting point of 90% purity, with β2GPI at 0.04 μM and the 3G4 antibody at 0.33 μM, this is a molar ratio of β2GPI to antibody of 0.12 (FIG. 9A). Even at a starting point of 80% purity, the same calculations show the in vivo concentration of β2GPI to be 4 μg/ml (0.08 μM), with the in vivo antibody concentration being 46 μg/ml (0.32 μM). This is still a molar ratio of β2GPI to antibody of only 0.25 (FIG. 9B). Subsequent studies, including sensitive western blotting of the antibody preparation (which could then identify the β2GPI band on a gel), confirmed that the 3G4 antibody was always at least 80-90% pure. These calculations of molar ratios of β2GPI to antibody of between 0.12 and 0.25 therefore add a quantitative perspective to the original explanation that the initial mouse data using the 3G4 antibody show that high levels of β2GPI are not necessary for anti-tumor activity.

### B. Low β2GPI Supports Cell Binding

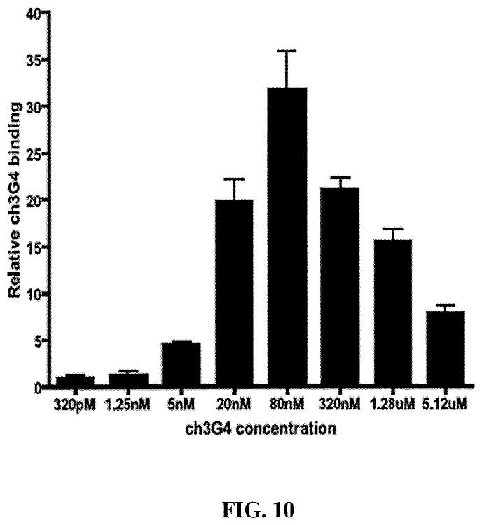

In a first study to analyze the β2GPI levels required for binding of 3G4-β2GPI complexes to PS on cells, ABAE cells were treated with LPC to induce PS exposure (Example IV) and then incubated with 40 nM purified human β2GPI and varying concentrations of the ch3G4 antibody (bavituximab). It was determined that relative ch3G4 binding increased in a concentration-dependent manner from 320 pM to an apparent peak of 80 nM ch3G4, which is an antibody to β2GPI ratio of 2:1 (FIG. 10). As shown in Table 1 (antibody MW, 145 kD; human β2GPI MW, 50 kD), the apparent peak binding in this study corresponds to a molar ratio of β2GPI to antibody of 0.5.

TABLE 1
Bavituximab and β2GPI in Cell Binding

| ch3G4 | ch3G4 | ch3G4 | β2GPI | β2GPI | β2GPI | Molar Ratio |
|---|---|---|---|---|---|---|
| (nM) | (μM) | (μg/ml) | (μg/ml) | (μM) | (nM) | β2GPI to Ab |
| 0.32 | 3.2 × 10⁻⁴ | 0.0464 | 2 | 0.04 | 40 | 125 |
| 1.25 | 1.25 × 10⁻³ | 0.1812 | 2 | 0.04 | 40 | 32 |
| 5 | 5 × 10⁻³ | 0.725 | 2 | 0.04 | 40 | 8 |
| 20 | 0.02 | 2.9 | 2 | 0.04 | 40 | 2 |
| 80 | 0.08 | 11.6 | 2 | 0.04 | 40 | 0.5 |
| 320 | 0.32 | 46.4 | 2 | 0.04 | 40 | 0.125 |
| 1280 | 1.28 | 185.6 | 2 | 0.04 | 40 | 0.03125 |
| 5120 | 5.12 | 742.4 | 2 | 0.04 | 40 | 0.0078125 |

The bell-shaped relationship between the concentration of ch3G4 and binding to cells with exposed PS (FIG. 10) further supports the formation of divalent ch3G42xβ2GPI complexes on the membrane surface, and suggests that monovalent ch3G4-β2GPI complexes form at very high antibody concentrations. At such concentrations, it is believed that competition between monovalent (non-binding) and divalent (binding) complexes caused a decrease in the amount of ch3G42xβ2GPI complex bound to the cells (also known as the “hook effect”).

The maximum relative binding in this study occurred at an antibody concentration of 80 nM, which is a β2GPI to antibody ratio of only 0.5 (FIG. 10; Table 1). This is a lower ratio than the predicted bivalent interactions as shown in FIG. 8, but is in general agreement with the ratios reported above for treating mice with the 3G4 antibody purified from the hybridoma. However, in this study, the exact density of PS on the cells is not known. More importantly, in the ch3G4 concentrations tested, saturated binding (plateauing) was not observed and further testing at ch3G4 concentrations between 20 and 80 nM, and between 80 and 320 nM, would have been informative. Nonetheless, even without such intermediate testing, it can be concluded that optimal antibody binding occurs at a molar ratio of β2GPI to antibody of between 0.125, 0.5 and 2. These in vitro numbers are in good agreement with those reported above for in vivo treatment using the hybridoma-purified 3G4 antibody, in which the molar ratios of β2GPI to antibody were between 0.12 and 0.25, depending on the antibody purity. This first in vitro study therefore also shows that low levels of β2GPI effectively support bavituximab binding to cells with exposed PS.

### C. Low β2GPI Supports PS Binding

In a follow-on study, the binding of the 2aG4 antibody (Example III) to PS was tested in an ELISA in the presence of varying concentrations of human β2GPI. Solutions containing a fixed amount of the 2aG4 antibody and increasing amounts of human β2GPI were prepared. Briefly 62.5 ng/ml (0.4 nM) of 2aG4 was added to 0.0032, 0.016, 0.08, 0.4, 2, 10 or 50 nM of human β2GPI in ovalbumin. The different 2aG4-β2GPI mixtures were added to PS microtiter plates and the molecules were allowed to bind for 1-2 h at 37° C. Unbound molecules were removed by washing with PBS. The secondary antibody was HRP-conjugated anti-human IgG in binding buffer. Plates were left for 1 hour at 37° C. before unbound antibody was removed by washing five times with PBS. TMB substrate was added to each well at a volume of 100 μl and left for 15 minutes at room temperature to allow the colorimetric reaction to occur. The reaction was stopped by addition of 100 μl of 2M H₂SO₄. Absorbances (optical density, OD) were read by a plate spectrometer at a wavelength of 450 nm within 30 minutes of adding the stop solution and analyzed using SoftMax Pro software (including subtracting the average OD of the control without β2GPI from the average OD of the test samples).

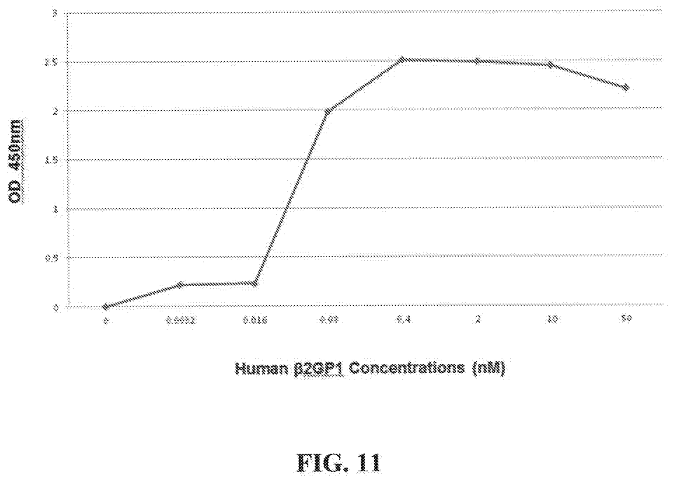

This study showed that 2aG4 antibody binding to PS started to plateau at a molar ratio of β2GPI to antibody of about 1, where both molecules were present at approximately 0.4 nM (FIG. 11). More precisely, as shown in Table 2 (antibody MW, 145 kD; human β2GPI MW, 50 kD), a molar ratio of β2GPI to antibody of 0.93 is effective in supporting antibody binding to plates coated with PS.

TABLE 2
Antibody and β2GPI in PS Binding

| 2aG4 | 2aG4 | β2GPI | Molar Ratio |
|---|---|---|---|
| (ng/ml) | nM | nM | β2GPI to Ab |
| 0 | 0 | 50 | — |
| 62.5 | 0.43 | 0 | — |
| 62.5 | 0.43 | 0.0032 | 7.42 × 10⁻³ |
| 62.5 | 0.43 | 0.016 | 0.037 |
| 62.5 | 0.43 | 0.08 | 0.186 |
| 62.5 | 0.43 | 0.4 | 0.93 |
| 62.5 | 0.43 | 2 | 4.65 |
| 62.5 | 0.43 | 10 | 23.26 |
| 62.5 | 0.43 | 50 | 116.28 |

Extending the observations from the first in vitro study above, this study showed that antibody binding to PS already reaches saturation at a low β2GPI to antibody molar ratio, after which a plateau is reached (a small loss of PS binding at 50 nM β2GPI was observed (FIG. 11), which is likely related to typical saturating effects commonly detected in ELISA assay formats). In the present study, the effective molar ratio of β2GPI to antibody was about 1 (0.93). Increasing the ratio of β2GPI to antibody to 5 or above did not result in improved binding (FIG. 11; Table 2).

A series of related studies were conducted testing the binding of bavituximab to PS in ELISAs in the presence of varying concentrations of human β2GPI in ovalbumin. Both bavituximab and β2GPI titrations were conducted. These studies also showed that low levels of β2GPI, including down to concentrations of 0.5 μg/ml, were effective in supporting a range of antibody concentrations in binding to plates coated with PS.

### D. Antibody Binding and Activity in Dilute Human Sera

Another series of studies was conducted to test the binding and functions of bavituximab to PS in varying dilutions of human sera. These included binding to PS in ELISAs, FACS analyses using PS-positive cells and functional assays in the form of an NFAT surrogate ADCC bioassay.

### 1. ELISA

An ELISA was conducted to test bavituximab binding to PS in varying percentages of six different individual human serum samples. Different human sera (from BioReclamationlVT, North America) were diluted in PBS to prepare a range of % human serum down to 0.1%. Bavituximab-HRP (Example XVI, A3) at 2 μg/ml was added to each % human serum solution. The different bavituximab-HRP mixtures were added to PS microtiter plates and allowed to bind for 1-2 h at 37° C. Plates were washed with PBS. TMB substrate was added to each well at a volume of 100 μl and left for 15 minutes at room temperature to allow the colorimetric reaction to occur. The reaction was stopped by addition of 100 μl of 2M H₂SO₄. Absorbances were read by a plate spectrometer at a wavelength of 450 nm within 30 minutes of adding the stop solution and analyzed using SoftMax Pro software.

### 2. FACS

Bavituximab binding to PS-positive cells (etoposide-treated HT 1080 cells) was tested using different percentages of FBS, as a source of bovine β2GPI, and measured by fluorescence-activated cell sorting (FACS), also known as flow cytometry.

Different percentages of FBS in PBS solutions were made and 10 μg/ml of bavituximab added to each solution. To induce PS exposure on the cell surface, HT 1080 cells were treated with 50 μM etoposide for 18 h. Cells were then incubated with the different bavituximab-percentage FBS solutions, followed by secondary antibody against bavituximab to allow visualization of antibody bound to cells via flow cytometry. Negative controls included cells not treated with etoposide, which will not expose PS on the surface, and PBS solutions lacking any FBS.

### 3. NFAT

Nuclear factor of activated T-cells (NFAT) is a general name applied to a family of transcription factors shown to be important in immune response. The NFAT signal transduction pathway and NFAT response elements (NFAT-RE) have been used in the development of assays, and commercially available kits, to monitor NFAT signal transduction pathways in cultured cells.

An NFAT bioassay has been developed for use with the bavituximab family of antibodies (Larson et al., 2013). Jurkat cells engineered to express the FcγRIIIa-V158 receptor on the cell surface (Promega) were also transfected with a genetic element containing the luciferase gene under the control of a minimal TATA promoter containing multiple NFAT-RE. These are the NFAT effector cells, which are co-cultured with PS-positive target cells. PS-targeting antibodies such as bavituximab bind to PS on the surface of target cells. The PS-targeting antibody's Fc region then binds to the FcγRIIIa-V158 receptor on the NFAT effector cells and signaling through the NFAT pathway is triggered. NFAT binds to the NFAT-RE and activates luciferase expression, which can be quantified. This NFAT assay is therefore a surrogate ADCC bioassay for bavituximab and other PS-targeting antibodies.

### 4. Results

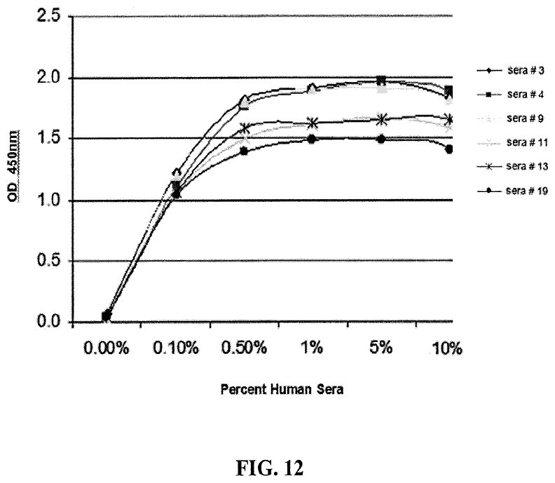

Exemplary results from the ELISA assay described above are presented in FIG. 12, which again show that dilute human sera, with low levels of β2GPI, effectively support antibody binding. As with the above PS ELISA using purified β2GPI (FIG. 11), there was some loss of PS binding at 50% and 100% human sera, which is related to typical saturating effects commonly detected in ELISA assay formats, particularly when using undiluted sera. Such effects are not observed in FACS assays, and bavituximab binding to cells with exposed PS was shown to be essentially the same in 50%, 75% and 100% fetal bovine serum.

From FIG. 12, it can be seen that bavituximab binding to PS in the ELISA started to plateau at about 1% human sera. As normal human sera contains, on average, 200 μg/ml β2GPI (Steinkasserer et al., 1991; Mehdi et al., 1999; Miyakis et al., 2004), 1% human sera contains about 2 μg/ml or 0.04 μM of β2GPI. FIG. 12 shows that bavituximab binding to PS was already at saturation at this concentration of β2GPI. As shown in Table 3 (antibody MW, 145 kD; human β2GPI MW, 50 kD), the 1% human sera in the ELISA corresponds to a molar ratio of β2GPI to antibody of 2.86. This generally accords with the rationale that each molecule of bavituximab needs to bind to two molecules of β2GPI to form a stable complex with PS on the cell surface (FIG. 8).

TABLE 3
Amount of β2GPI in human Sera for PS Binding

| Bavi | Bavi | Human | β2GPI | β2GPI | Molar Ratio |
|---|---|---|---|---|---|
| (μg/ml) | (μM) | Sera % | (μg/ml) | (μM) | β2GPI to Ab |
| 2 | 0.014 | 10 | 20 | 0.4 | 28.57 |
| 2 | 0.014 | 5 | 10 | 0.2 | 14.28 |
| 2 | 0.014 | 1 | 2 | 0.04 | 2.86 |
| 2 | 0.014 | 0.5 | 1 | 0.02 | 1.43 |
| 2 | 0.014 | 0.1 | 0.2 | 0.004 | 0.28 |
| 2 | 0.014 | 0 | 0 | 0 | — |

As shown in FIG. 12, even at 0.5% human sera, bavituximab binding to PS is approaching the plateau, most particularly for serum sample #13, and this corresponds to a molar ratio of β2GPI to antibody of 1.43 (Table 3). Results from the NFAT surrogate ADCC bioassay also indicated that molar ratios of β2GPI to antibody in this general range were effective to support bavituximab function. For example, although the study was not designed to identify optimal ratios for bavituximab activity, a molar ratio of (bovine) β2GPI to antibody of 1.9 was shown to effectively support bavituximab activity in the NFAT assay.

In summary, the present example shows that molar ratios of β2GPI to antibody from as low as 0.12 to 2.86 support antibody binding to PS and PS-positive cells, facilitate activity in functional assays and permit effective treatment of mice with tumors. In light of all the above data, and taking a precautionary approach, it was deduced that to maximize bavituximab binding and function, a molar ratio of β2GPI to antibody should be about 2.86 (Table 3), but that it does not need to be higher than about 3.

### Example VI

### Pharmacokinetics of Bavituximab in Clinical Studies

This example concerns pharmacokinetics of bavituximab when administered to human subjects having diseases in which PS is a marker, particularly cancer and viral infections. The clinical experience is shown to be consistent with the pre-clinical modelling, as described above.

### A. Initial Phase I Study

A Phase I, multicenter, open-label, dose escalation study was conducted to evaluate the safety, tolerability and pharmacokinetics (PK) of bavituximab when administered intravenously (bavituximab monotherapy) to 26 patients with refractory advanced solid tumors. Patients were enrolled into four sequential dose-escalation cohorts (0.1, 0.3, 1 or 3 mg/kg bavituximab weekly) with two dosing schedules. In the 0.1 mg/kg and 0.3 mg/kg cohorts, patients received bavituximab on days 0, 28, 35 and 42; and in the 1 mg/kg and 3 mg/kg cohorts, patients were administered bavituximab on days 0, 7, 14 and 21.

The upper dose of 3 mg/kg weekly was selected based on preclinical modeling (Example V) and experience in other patient populations. In extensive animal model studies subsequent to those of Example II, maximal efficacy was achieved at antibody doses of 0.5 mg/kg 3 times weekly, yielding a Cₘₐₓ of 5.5 μg/ml with a half-life of 48 hours and a simulated average blood concentration of 2 μg/ml over the course of treatment. Beyond such a dose, PS binding by bavituximab was presumably saturated, based on observations of the concentration at which binding of bavituximab to PS-positive cells becomes saturated in vitro (Example V).

Samples were collected from patients in the 0.1 and 0.3 mg/kg dose cohorts before the study, on days 0, 1, 2, 4, 7, 10, 14, and every 7 days from days 21 to 70. Samples were collected from patients in the 1 and 3 mg/kg dose cohorts before the study, on days 0, 1, 2, 4, 7, 14, 21, 22, 23, 25, and every 7 days from days 28 to 56. Bavituximab blood levels were determined by a validated ELISA.

Table 4 presents a summary of the mean (coefficient of variation, CV) PK parameters of bavituximab following single-dose administration (day 0) and multiple-dose administration (day 21) in this Phase I trial, including maximum concentration (Cₘₐₓ), clearance (CL), half-life (t_{1/2}) and area under plasma concentration-time curve from time zero to infinity (AUC_{inf}).

TABLE 4
Pharmacokinetic Parameters of Bavituximab in Phase I Trial

| | | Mean (CV %) | | | |
|---|---|---|---|---|---|
| Dose | | Cₘₐₓ | CL | t_{1/2} | AUC_{inf} |
| (mg/kg) | N= | (μg/ml) | (ml/h/kg) | (h) | (d μg/ml) |
| | Day 0 | | | | |
| 0.1 | 8 | 2.11 (27.3) | 1.10 (48.7) | 43.9 (48.5) | 113 (50.1) |
| 0.3 | 6 | 5.13 (42.4) | 1.39 (34.3) | 39.8 (34.1) | 241 (39.8) |
| 1.0 | 6 | 16.6 (30.9) | 1.14 (36.7) | 40.3 (20.2) | 966 (30.0) |
| 3.0 | 6 | 56.4 (25.8) | 1.34 (72.2) | 37.2 (34.5) | 3,017 (50.3) |
| | Day 21 (for 1 and 3 mg/kg) | | | | |
| 1.0 | 6 | 18.7 (31.8) | 1.12 (52.1) | 46.8 (38.4) | 1,053 (38.0) |
| 3.0 | 4 | 59.6 (27.6) | 1.51 (61.4) | 46.0 (44.4) | 2,672 (63.4) |

As shown in Table 4, following single-dose administration, it was determined that the mean half-life of bavituximab ranged from 37.2 to 43.9 hours. On day 0, the mean maximum serum concentration (Cₘₐₓ) ranged from 2.11 to 56.4 μg/ml (depending on dose) at the median time after administration when the maximum serum concentration was reached (Tₘₐₓ) (values ranging from 2.04 to 3.73 hours). For bavituximab administered at 3 mg/kg, the maximum serum concentration was 56.4 μg/ml. For the study overall, the bavituximab half-life ranged from 37 to 47 hours. No maximum tolerated dose was reached in this study.

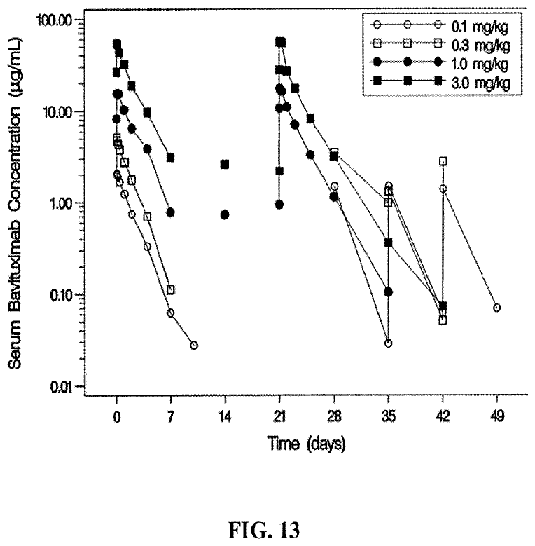

Bavituximab exhibited linear single-dose (day 0) and multiple-dose (days 21 or 42) PK characteristics (FIG. 13). Bavituximab did not exhibit appreciable accumulation or time-dependent PK differences following multiple-dose administration. In summary, this study showed that bavituximab was well tolerated at doses ranging up to 3 mg/kg weekly and the pharmacokinetics support a weekly dosing regimen. In particular, it was determined that at the dose of 1 mg/kg, the bavituximab concentration remained above 2 μg/ml, the predicted therapeutic threshold based on preclinical modeling, for 6 days; and at the dose of 3 mg/kg, the bavituximab concentration remained above this 2 μg/ml for 7 days (FIG. 13). The dose of 3 mg/kg weekly was therefore selected for future use in oncology.

### B. Further Pharmacokinetic Studies

In addition to the above Phase I trial, the PK of bavituximab given as a single dose, weekly or twice weekly infusion (60-90 minutes) has now been evaluated in over 120 patients across several other clinical studies in patients with cancer or viral infections. It was confirmed that bavituximab exhibits linear single-dose and multiple-dose PK characteristics at doses ranging from 0.1 to 6 mg/kg, with no evidence of appreciable accumulation of bavituximab or time-dependent PK differences. The median Tₘₐₓ was shown to occur within the first 2 to 3 hours following the end of the infusion. Serum bavituximab concentrations decline in an apparent mono-exponential or bi-exponential first-order manner. The more rapid distribution phase, where observed, is essentially complete within 6 hours and the terminal elimination half-life is approximately 1 to 2 days (21.9 to 46.8 hours).

### 1. PK in Viral Infections

Bavituximab PK characteristics are generally similar in patients with cancer and chronic viral infections, as tested in patients chronically infected with HCV, with and without HIV.

A Phase I, open-label, single center, dose escalation study evaluated a single intravenous infusion of bavituximab in patients chronically infected with HCV (Example VII, A). As shown in Table 5, it was found that the observed concentrations of bavituximab were very consistent with the predictions from the PK modeling data.

TABLE 5
Predicted and Measured Bavituximab Concentrations

| Parameters | Values | | | | |
|---|---|---|---|---|---|
| Doses (mg/kg) | 0.1 | 0.3 | 1 | 3 | 6 |
| Predicted Cₘₐₓ (μg/ml) | 2.2 | 6.5 | 21.8 | 65.4 | 130.8 |
| Observed Cₘₐₓ (μg/ml) | 2.5 | 5.7 | 24.3 | 75.8 | 135.0 |

In the corresponding Phase Ib, multi-center, open-label, non-randomized, escalating repeat-dose study in patients with chronic HCV, analysis of the PK data showed linear single-dose PK characteristics on day 0 and linear multiple-dose characteristics on day 10 at all dose levels, with no evidence of accumulation of bavituximab or time-dependent PK differences after 2 weeks dosing.

In the Phase Ib study in patients co-infected with chronic HCV and HIV (Example VII, C), bavituximab exhibited linear single-dose PK characteristics on day 0 and linear multiple-dose PK characteristics on day 49 following once weekly administration at doses ranging from 0.3 to 6 mg/kg. Bavituximab did not exhibit time-dependent PK differences or accumulation following multiple-dose administration once weekly for 8 weeks.

### 2. PK in Combination Therapies

Importantly, when bavituximab and other drugs (particularly chemotherapeutic agents) were given in combination, there did not appear to be any clinically relevant pharmacokinetic interactions for either of the drugs. This includes when bavituximab and docetaxel were given in combination.

In this regard, a Phase Ib, multi-center, open-label, non-randomized study first evaluated the safety, tolerability and PK of weekly intravenous administration of 3 mg/kg bavituximab when used in combination with gemcitabine, paclitaxel plus carboplatin or docetaxel in patients with refractory advanced solid tumors. It was determined that there were no significant differences in any measurable parameter among the three treatment groups following a single-dose (day 0) or multiple-dose bavituximab administration (day 21). Evaluation of Cₘₐₓ and AUC indicated that no accumulation of bavituximab following multiple-dose administration once weekly for eight weeks.

Within a Phase II, randomized, double-blind, placebo-controlled study evaluating bavituximab plus docetaxel in patients with previously treated locally advanced or metastatic non-squamous NSCLC (Example XIII), a subset of the overall study population (6 patients per arm) also participated in a PK sub-study to investigate any drug-drug interactions between bavituximab and docetaxel. Additional blood draws were performed for these patients during cycles 1 and 2 at specified time points. No clinically relevant pharmacokinetic drug-drug interaction was observed for bavituximab with docetaxel. In addition, docetaxel exhibited similar pharmacokinetic characteristics with or without the administration of bavituximab. Thus, no clinically relevant pharmacokinetic drug-drug interaction was observed for docetaxel with bavituximab in these patients.

### Example VII

### Treating Viral Infections in Patients using Bavituximab

In this example, data are presented to exemplify some of the clinical experience in treating viral infections in patients using bavituximab, including bavituximab in combination with ribavirin. Data are also presented to show that, at the selected clinical dose, administration of bavituximab does not appreciably reduce β2GPI levels in human subjects.

### A. Phase I Studies in HCV Patients

Bavituximab was first evaluated in Phase I, open-label, dose escalation studies and Phase Ib, open-label, escalating repeat-dose studies in patients chronically infected with hepatitis C virus (HCV). These studies concerned the safety, tolerability, PK profile, viral kinetics, maximum tolerated dose (MTD) and maximum effective dose (MED) of bavituximab. Doses of 0.1, 0.3, 1, 3 and 6 mg/kg were administered in Phase I (30 patients; successive cohorts of 6 patients), and doses of 0.3, 1, 3 and 6 mg/kg were administered in Phase Ib (24 patients; four cohorts of 6 patients).

In the Phase I and Phase Ib studies in HCV patients, all dose levels of bavituximab were well tolerated. In Phase I, transient reductions in viral load suggestive of anti-viral activity were observed at all dose levels. In Phase Ib, small decreases in viral load resulted after treatment with bavituximab at doses of 0.3, 1 and 6 mg/kg; those decreases were often transient, but at least one patient in each cohort had a sustained decrease in viral load. Notably, at doses of bavituximab of 3 mg/kg, consistent decreases in HCV were demonstrated throughout study treatment and follow-up.

### B. Bavituximab Does Not Deplete β2GPI

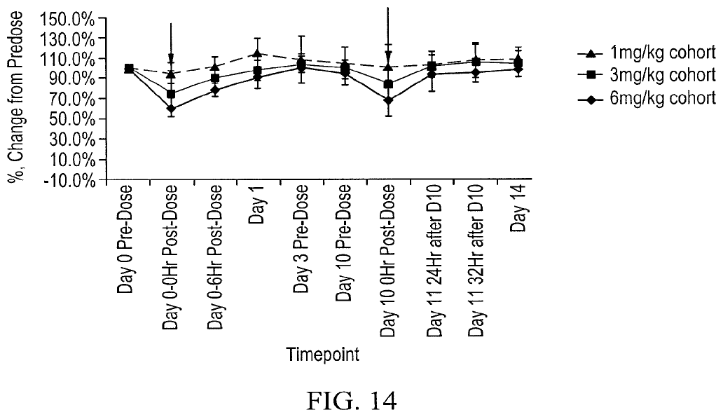

The Phase Ib study described above also measured levels of β2GPI in the patients, to determine whether administration of bavituximab altered β2GPI levels in these human subjects. The results are depicted in FIG. 14. In patients receiving 1 mg/kg bavituximab, β2GPI levels were virtually unchanged. A transient reduction (20 to 25%) in serum levels of β2GPI was observed in patients receiving 3 mg/kg bavituximab. However, such a reduction was not statistically significantly changed from the pre-dose levels (FIG. 14). Indeed, at the 3 mg/kg bavituximab dose, β2GPI levels remained within the normal range and returned to the pre-treatment level within 24 hours. In contrast, in patients receiving 6 mg/kg bavituximab, β2GPI levels were significantly reduced (p<0.02) (FIG. 14). At the 6 mg/kg dose, β2GPI levels fell by 40% relative to the pretreatment levels, to approximately the lower limit of the normal range. Nonetheless, even in human subjects treated with bavituximab at 6 mg/kg, β2GPI recovered to baseline levels in 3 days.

These data therefore validated the selection of the 3 mg/kg dose of bavituximab for use in humans. This dose was determined to be the maximal dose at which bavituximab and β2GPI were present together at concentrations effective to allow the bavituximab-β2GPI complex to form and bind to PS exposed on cells in the disease site without depleting plasma β2GPI levels. However, the data also show that any reductions in β2GPI during bavituximab treatment are only temporary and that β2GPI levels are restored within 3 days.

### C. Phase I Study in HCV-HIV Patients

A separate Phase Ib, multi-center, open-label, non-randomized, dose-escalating, repeat-dose study was conducted to evaluate bavituximab in patients co-infected with chronic HCV (majority of HCV genotype 1) and human immunodeficiency virus (HIV). The primary objectives were to determine the safety, tolerability, PK profile, viral kinetics, MTD and/or MED. The study involved 16 scheduled visits over approximately 16 weeks. Bavituximab was administered to successive cohorts of patients at the following doses: 0.3 mg/kg, six patients; 1 mg/kg, six patients; 3 mg/kg, nine patients; and 6 mg/kg, six patients. Patients received intravenous bavituximab weekly for 8 weeks. Dose escalation proceeded after all patients in the cohort had completed the first 4 weeks of dosing with no thrombotic events classified as serious adverse events (SAEs).

The median baseline HCV viral load was 6.76 log₁₀ and the median baseline for HIV was 3.99 log₁₀. Plasma viral loads of HCV and HIV were measured at specific time points during the study. When treated with bavituximab at all dose levels, several patients in each treatment group exhibited transient antiviral activity (maximum reduction in HCV and/or HIV viral load of ≥0.5 log₁₀ from baseline).

### D. Phase II Study in HCV Patients

A Phase II, multi-center, randomized, active-control study was conducted to evaluate bavituximab in combination with ribavirin for the initial treatment of chronic HCV (genotype 1) infection. The primary endpoint was the proportion of patients who showed an early virological response (EVR) at Study Week 12, with an EVR being defined as equal to or greater than a 2-log₁₀ international unit (IU) reduction in HCV RNA level. Safety was included amongst the secondary endpoints.

Patients underwent a screening/washout period of up to 28 days followed by randomization (in a 1:1:1 ratio) to receive 0.3 or 3 mg/kg weekly bavituximab infusion or pegylated interferon alpha-2a (pegylated interferon, also referred to as PEG-IFNα-2a) subcutaneous injection for 12 weeks, all with twice-daily oral ribavirin 1000 mg (weight<75 kg) or 1200 mg (weight≥75 kg). Patients who showed an EVR after 12 weeks received off-study treatment with pegylated interferon plus ribavirin up to a 48 week course.

A total of 66 patients (38 males and 28 females) with a mean age of 39.1 years were enrolled to the study. Twenty-two patients each received 0.3 mg/kg bavituximab, 3 mg/kg bavituximab and pegylated interferon. The median number of 0.3 and 3 mg/kg bavituximab doses received was 12 doses each, and the mean duration of treatment was 78 and 75 days, respectively.

In this study, a gradual viral reduction over 12 weeks was seen in some patients treated with bavituximab plus ribavirin. Interestingly, an EVR was seen in twice as many patients treated with the lower dose of bavituximab (0.3 mg/kg), as opposed to the higher dose of 3 mg/kg bavituximab (18% vs. 9%). Although the EVR rate was higher in patients receiving pegylated interferon than bavituximab at either dose, bavituximab displayed a more favorable safety profile; almost twice as many patients in the pegylated interferon arm reported AEs compared to either bavituximab-containing arms.

### Example VIII

### Treating Breast Cancer Patients with Bavituximab and Paclitaxel

Turning to clinical cancer treatment, the present example provides data from the treatment of patients with HER2-negative metastatic breast cancer using bavituximab in combination with the taxane, paclitaxel.

In a single-center, investigator-sponsored study, 14 patients with HER2-negative metastatic breast cancer received bavituximab at 3 mg/kg weekly in combination with paclitaxel (80 mg/m²) given on days 1, 8 and 15 in 4-week cycles. Bone pain, fatigue, headache and neutropenia were the most common adverse events (AEs). Manageable infusion-related reactions were the most common AE related to bavituximab. Bavituximab showed no evidence for increased thrombogenicity. Treatment resulted in an overall response rate (ORR) of 85%, with 2 patients having a complete response, and a median progression-free survival (PFS) of 7.3 months (95% CI: 2.8, 10.8).

In summary, this study showed that bavituximab in combination with paclitaxel is well tolerated for the treatment of patients with metastatic breast cancer, with promising results observed in terms of clinical response rates (RRs) and PFS.

### Example IX

### Treating Breast Cancer Patients with Bavituximab and Paclitaxel-Carboplatin

This example reports results from a Phase II, open-label, single arm study evaluating the safety and efficacy of bavituximab plus paclitaxel and carboplatin in patients with locally advanced or metastatic breast cancer, unrestricted by hormone or HER2 status.

This Phase II study utilized a Simon 2-stage design. Fifteen patients were enrolled into Stage A and the trial was expanded to an additional 31 patients in Stage B, for a total of 46 patients. The primary objective was to determine the overall response rate (ORR), defined as complete response (CR) plus partial response (PR), CR+PR. Secondary objectives included time to tumor progression, duration of response (DOR or DR), overall survival (OS) and safety.

Bavituximab (3 mg/kg) was given weekly until disease progression, in combination with carboplatin (at a dose of AUC=2) and paclitaxel 100 mg/m² on days 1, 8, and 15 of a 28-day cycle for up to 6 cycles. Sixteen of the 46 patients (34.8%) were treatment naive.

The most common Grade 4 treatment-emergent adverse event (TEAE) was neutropenia (12 patients, 26.1%), which is the expected incidence in patients treated with the chemotherapies used in this study. The most common Grade 3 TEAEs were leukopenia (11 patients, 23.9%), neutropenia (9 patients, 19.6%), and anemia (5 patients, 10.9%). These are also the expected incidences in patients treated with the chemotherapies used in this study.

An objective response per Response Evaluation Criteria in Solid Tumors (RECIST) occurred in 34 of 46 patients (73.9%); 5 of 46 patients (10.9%) had a CR and 29 patients (63.0%) had a PR. The median duration of response (DOR) was 3.7 months (95% confidence interval [CI]: 3.1, 5.8) and the median PFS was 6.9 months (95% CI: 5.6, 7.7). At study closure, the median OS was determined to be 23.2 months (95 CI: 553 days to ‘not determined’). These results are very encouraging for the ongoing development of bavituximab, particularly in combination therapies.

### Example X

### Treating Breast Cancer Patients with Bavituximab and Docetaxel

The present example reports results from another Phase II, open-label, single arm study evaluating the safety and efficacy of bavituximab, this time in combination with docetaxel in patients with locally advanced or metastatic breast cancer.

This trial was also a Phase II, multicenter trial utilizing a Simon 2-stage design. Fifteen patients were enrolled into Stage A and the trial was expanded to an additional 31 patients in Stage B, for a total of 46 patients. The primary objective was to determine the ORR (CR+PR). Secondary objectives included time to tumor progression, DOR, OS and safety.

Bavituximab (3 mg/kg) was given weekly until progression, in combination with docetaxel (35 mg/m²), given on days 1, 8, and 15 of planned 4-week cycles for up to 6 cycles. All patients received one prior chemotherapy regimen. Of the most common TEAEs reported, only fatigue, headache, back pain and hypertension were Grade≥3.

In this study, it was determined that an objective response occurred in 28 of 46 patients (60.9%); 5 of 46 patients (10.9%) had a CR and 23 of 46 patients (50.0%) had a PR. The median DOR of 6.1 months (95% CI: 5.7, 7.5) and median PFS of 7.4 (95% CI: 6.1, 9.1) months. At the time of final analysis, median OS was approximately 20.7 months (95% CI: 16.1 months to ‘not determined’). These data provide strong support for the further development of bavituximab, including in combination therapies with docetaxel.

### Example XI

### Treating Liver Cancer Patients with Bavituximab and Sorafenib

In this example, data are presented from the treatment of patients with advanced hepatocellular carcinoma (HCC) using bavituximab in combination with sorafenib.

A Phase II, single institution study of bavituximab and sorafenib in advanced hepatocellular carcinoma (HCC) was conducted. Patients received weekly bavituximab at 3 mg/kg intravenously (IV) and 400 mg sorafenib by mouth, two times per day (PO BID) until radiologic progression. Secondary endpoints included overall survival (OS), disease specific survival, 4 month progression free survival, safety and response rate. The study accrued 38 patients.

In related translational data from six patients in this study, it was determined that half of the patients evaluated had an increase in tumor fighting immune cells following one cycle of bavituximab treatment, similar to what has been shown for related PS-targeting antibodies in multiple preclinical cancer models. In addition, the increase in immune response was associated with patients that remained on study treatment for longer time periods, suggestive of a clinically meaningful anti-tumor immune response. Three of the six patients evaluated had increased infiltration of activated tumor-fighting T-cells (CD8) into the tumor microenvironment, which correlated with a prolonged time to disease progression. In addition, these responding patients initially expressed lower levels of PD-1 positive cells, an established marker of T-cell activation and disease outcome, prior to the initiation of therapy that was followed by a measurable rise post bavituximab treatment.

Clinically, there were no grade 4 or 5 adverse events recorded. The most common all grade events were diarrhea (32%), fatigue (26%) and anorexia (24%). The median OS (mOS) was 6.2 months. Two patients achieved partial response and the four month PFS was 61%.

These results demonstrated that bavituximab and sorafenib were well tolerated in patients with advanced HCC, with no indications of autoimmune adverse events that have been seen with other checkpoint immunotherapies. The clinical outcomes of time to progression, disease control rate and 4-month progression-free survival are encouraging, especially in this heavily pretreated patient cohort with very poor prognosis due to their unfavorable disease biology including a high rate of macrovascular invasion.

### Example XII

### Treating Pancreatic Cancer Patients with Bavituximab and Gemcitabine

In the present example, data are presented from the treatment of patients with previously untreated stage IV pancreatic cancer using gemcitabine in combination with bavituximab.

This study (PPHM 1002) was a Phase II, randomized, open-label study to evaluate gemcitabine when administered with or without bavituximab in patients with previously untreated stage IV pancreatic cancer. The primary objective was to compare the OS of patients among the treatment arms. Secondary objectives included comparing PFS, ORR, DR and safety.

Enrolled patients were randomized in a 1:1 ratio to receive study treatment of gemcitabine alone or gemcitabine with weekly 3 mg/kg bavituximab. Gemcitabine (1000 mg/m²) was given on days 1, 8, and 15 of each 28-day cycle (4 weeks) until disease progression or unacceptable toxicities. A total of 70 patients were enrolled to the study. In general, the patient population had very extensive disease burden, which may have reduced the response in both arms.

The most common TEAEs for the bavituximab plus gemcitabine treatment group were nausea (44.1%), anemia (35.3%), and fatigue, constipation and anorexia (each occurring in 32.4% of patients). Three (9.1%) patients randomized to gemcitabine only had Grade 5 (fatal) events (sudden death [1 patient], liver abscess [1 patient], and cardiac arrest [1 patient]). None of the Grade 5 (fatal) events occurred in the gemcitabine plus bavituximab group.

Although most efficacy endpoints were comparable across treatment groups, there was a numerically higher response rate and survival probability at 1 year in the bavituximab and gemcitabine group. At study closure, the median overall survival (95% CI) was 5.2 (4.0 to 6.3) months in the gemcitabine only treatment group and 5.6 (4.7 to 7.0) months in the bavituximab plus gemcitabine treatment group. These outcomes for the addition of bavituximab are encouraging, particularly in this patient population with very extensive disease burden.

After the Phase III trial of Example XIV, and the functional β2GPI analyses of Example XVII, showing that functional β2GPI levels correlate with treatment outcomes, stored samples from the present Phase II trial were also tested for functional β2GPI. Results from these analyses, as reported in Example XVIII, strengthen the finding that levels of functional β2GPI are a biomarker for successful bavituximab treatment.

### Example XIII

### Phase II Trial of Bavituximab and Docetaxel in NSCLC Patients

Building on the Phase I and single arm Phase II experience, the present example concerns a Phase II trial testing bavituximab plus docetaxel in patients with previously-treated Stage IIIb/IV non-squamous non-small cell lung cancer (NSCLC).

This study (PPHM 0902) was a Phase II, randomized, double-blind, placebo-controlled trial evaluating bavituximab plus docetaxel in patients with previously treated locally advanced or metastatic non-squamous NSCLC. The primary objective of this study was to compare the ORR (CR+PR) among the treatment arms. Secondary objectives included comparing PFS, DR, OS, safety and PK.

Patients were randomized in a 1:1:1 ratio to receive docetaxel plus placebo, docetaxel plus bavituximab at 1 mg/kg, or docetaxel plus bavituximab at 3 mg/kg. Docetaxel 75 mg/m² was given on day 1 of each 21-day cycle for up to 6 cycles, and placebo or the assigned dose of bavituximab was given weekly. Patients continued to receive assigned blinded treatment (placebo, 1 mg/kg bavituximab or 3 mg/kg bavituximab) weekly until progression or toxicity.

A subset of the overall study population (6 patients per arm) participated in a PK sub-study to investigate the drug-drug interaction between bavituximab and docetaxel. Additional blood draws were performed for these patients during Cycles 1 and 2 at specified time points.

A total of 121 patients (76 males and 45 females) with a mean age of 60.0 years were enrolled in the study. Study treatment was unblinded following an Independent Data Monitoring Committee (IDMC) meeting, in which it was determined that the primary endpoint of ORR had been reached and unblinding of study treatment was thus recommended. Additionally, no safety concerns or issues were identified by the IDMC.

After study unblinding, a labeling error by the package and labeling vendor was discovered involving the placebo and 1 mg/kg arms. An investigation summary was submitted to the Food and Drug Administration (FDA) and data from patients dosed with placebo or 1 mg/kg bavituximab were pooled to form a combined control arm for exploratory analyses and comparison to the 3 mg/kg bavituximab group.

Overall, no significant difference was observed in the incidence of AEs by toxicity grade between the treatment groups. No notable differences were observed in SAEs between treatment groups. Three patients (3.8%) in the combined control group and 2 patients (5.0%) in the 3 mg/kg bavituximab with docetaxel group had Grade 5 (fatal) events. The combined control patients with fatal events included 1 patient with sepsis, 1 patient with a cerebrovascular accident, and 1 patient experiencing both pneumonia and pseudomonal sepsis. In the 3 mg/kg bavituximab plus docetaxel group, 1 patient had fatal sepsis unrelated to bavituximab, and 1 patient had an event of failure to thrive, also unrelated to bavituximab.

A summary of efficacy endpoints is presented in Table 6, in which the analyses are based on the Intend-To-Treat (ITT) population and central review data. All endpoints (ORR, PFS, and OS) demonstrated trends towards superiority for bavituximab 3 mg/kg, compared to the combined control arm (placebo or 1 mg/kg bavituximab). The ORR was approximately 50% greater for bavituximab 3 mg/kg compared to the combined group. Although median PFS was similar between the combined groups and the 3 mg/kg bavituximab group, median OS was approximately 60% longer for patients receiving bavituximab 3 mg/kg. In particular, the patients treated with 3 mg/kg bavituximab plus docetaxel had a mOS of 11.7 months vs. only 7.3 months mOS for the patients in the combined arm (HR=0.66).

TABLE 6
Summary of Efficacy Analysis Based in Phase II Trial

| | Placebo + | Bavituximab |
|---|---|---|
| Efficacy Measure | Bavituximab 1 mg/kg | 3 mg/kg |
| N= | 80 | 41 |

Overall Response Rate (CR + PR)

| | | | | |
|---|---|---|---|---|
| N (%) | 9 | (11.3) | 7 | (17.1) |

| | | |
|---|---|---|
| 95% CI | (4.3, 18.2) | (5.6, 28.6) |

Progression-Free Survival

| | | | | |
|---|---|---|---|---|
| Median in Days (Months) | 119 | (3.9) | 127 | (4.2) |

| | | |
|---|---|---|
| 95% CI (Days) | (79, 126) | (82, 197) |

Overall Survival

| | | | | |
|---|---|---|---|---|
| Number of Deaths (%) | 60 | (75.0) | 22 | (53.7) |
| Median in Days (Months) | 221 | (7.3) | 355 | (11.7) |

| | | |
|---|---|---|
| 95% CI (Days) | (169, 367) | (157, 525) |

Subsequent to the Phase III trial of Example XIV, and the analyses of functional β2GPI in Example XVII, which showed that functional β2GPI levels correlate with treatment outcomes, stored samples from the present Phase II trial were also tested for functional β2GPI. Results from these analyses, which are described in Example XVIII, further validate that levels of functional β2GPI are a biomarker for successful bavituximab treatment.

### Example XIV

### Phase III Trial of Bavituximab and Docetaxel in NSCLC Patients

As reported in the previous examples, the overall results from Phase I and Phase II studies have demonstrated a clinically meaningful treatment effect of bavituximab. Based on such results, and particularly on the double-blind Phase II trial described above, a Phase III trial was undertaken and the present example describes the Phase III trial and the resulting data.

The Phase III trial (PPHM 1202) was a randomized, double-blind, placebo-controlled multicenter trial of bavituximab plus docetaxel in patients with previously-treated Stage IIIb/IV non-squamous non-small cell lung cancer (NSCLC). This global, double-blind Phase III trial was initiated in 2012. Selection criteria were for patients with Stage IIIb/IV non-squamous NSCLC who progressed on platinum-doublet chemotherapy (should have progressed on appropriate targeted therapy if known EGFR or ALK mutation), with ECOG PS 0-1 and prior immunotherapy allowed. The trial accrued 597 such patients in a 1:1 ratio to receive up to six 21-day cycles of docetaxel (at 75 mg/m²) in combination with either weekly 3 mg/kg bavituximab (bavituximab plus docetaxel) or placebo (docetaxel alone) until progression or toxicity. The primary endpoint was overall survival (OS) and secondary endpoints included objective response rate (Independent Central Review, ICR), progression-free survival (ICR), safety, PK, Quality of Life (LCSS) and exploratory biomarkers, including immune correlates. The baseline characteristics of the selected patients are shown in Table 7, in which the ‘Placebo’ column refers to patients treated with docetaxel alone and the ‘Bavituximab’ column refers to patients treated with bavituximab plus docetaxel.

TABLE 7
Baseline Characteristics of Patients in Phase III Trial

| | Placebo | Bavituximab |
|---|---|---|
| | n = 300 | n = 297 |
| Median Age, yrs (Range) | 62 (30-82) | 63 (37-85) |
| >75 (%) | 5 | 8 |
| Male/Female % | 61/39 | 60/40 |
| Disease stage % | | |
| Stage IIIb | 5 | 5 |
| Stage IV | 95 | 95 |
| Current/former smoker % | 75 | 79 |
| Genetic mutation % | | |
| EGFR | 8 | 12 |
| ALK | 2 | 2 |
| Other | 14 | 9 |
| None | 52 | 52 |
| Unknown or not tested | 26 | 28 |
| Performance status % | | |
| 0 | 29 | 32 |
| 1 | 70 | 66 |
| Prior therapy % | | |
| Maintenance and/or targeted therapy | 57 | 57 |
| Immunotherapy | 4 | 2 |
| Baseline β2GP1 ≥ 200 μg/ml % | 49 | 56 |

### A. Safety

With 70% of the targeted OS events reached, the median OS (mOS) was assessed (see below). Throughout the study, it was determined that the safety profile was generally similar between groups. The treatment and safety summary has recently been published (Palmero et al., 2017). As reported therein, the safety profile of the combination of bavituximab with docetaxel is similar to placebo plus docetaxel. Grade 3 or higher adverse events occurred in 68% of patients in the bavituximab plus docetaxel group and 60% of those in the docetaxel alone group. Treatment-related AEs reported in greater than 15% of patients were reported recently (Palmero et al., 2017). In addition, treatment-related Grade 3/4 febrile neutropenia was slightly higher for bavituximab plus docetaxel (8%) than for docetaxel alone (5%). Note that the number of patients treated with docetaxel alone (Placebo, n=300) in the baseline characteristics of Table 7 is based on the ITT population, i.e., including all randomized patients, whereas the number of patients treated with docetaxel alone (Placebo, n=299) as published in Palmero et al., 2017 is based on the Safety population, i.e., including all randomized patients who received treatment.

### B. Efficacy

With 70% of the targeted OS events reached, the mOS was 10.7 months (95% confidence interval [CI], 8.6-11.5) among 297 patients in the bavituximab plus docetaxel group and 10.8 months (95% CI, 9.2-12.6) among 300 patients in the docetaxel alone group (hazard ratio (HR) for death, 1.10 (0.89, 1.37)). Progression-free survival (PFS) was also similar in the two arms when 70% of the targeted OS events were reached, with a median PFS of 4.1 months for the bavituximab plus docetaxel group and 3.9 months for the docetaxel alone group. Subsequent immunotherapy was received by about 15% of the patients in the study, evenly distributed between the bavituximab plus docetaxel arm and the docetaxel alone arm (see Example XIX).

With 12 months follow-up from the last patient randomized and about 85% of the targeted OS events reached, the median OS is 10.5 months (95% confidence interval [CI], 8.4-11.9) among 297 patients in the bavituximab plus docetaxel group and 10.9 months (95% CI, 9.2-12.1) among 300 patients in the docetaxel alone group (HR, 1.06; P=0.533). PFS at this stage was 4.2 months (95% CI, 3.9-4.6) in the bavituximab plus docetaxel group and 4.1 months (95% CI, 3.2-4.8) in the docetaxel alone group (HR, 1.02; P=0. 876). The ORR at this stage was 15% in the bavituximab plus docetaxel group vs. 11% in the docetaxel alone group (odds ratio, 0.7; P=0.15).

The efficacy analysis (ITT) at this stage is listed in Table 8, in which the P-value is based on the two-sided stratified Cochran-Mantel-Haenszel exact method. Stratification factors include disease stage (IIIB vs. IV), geographic region (North America, Europe, Rest of World), previous maintenance and/or targeted therapy (Yes vs. No).

TABLE 8
Phase III Trial, Efficacy Analysis

| | Placebo | | Bavituximab | |
|---|---|---|---|---|
| Efficacy Measure | n = 300 | | n = 297 | |
| Overall Survival (OS) | | | | |
| Number of Patients who | 212 | (70.7%) | 206 | (69.4%) |
| Died (%) | | | | |
| Median in Months | 10.9 | (9.2 to 12.1) | 10.5 | (8.4 to 11.9) |
| (95% CI) | | | | |
| HR (95% CI); p-value | 1.06 (0.88-1.29); p = 0.533 | | | |
| Objective Response Rate | | | | |
| (ORR) - CR + PR | | | | |
| % of Patients (95% CI) | 11 | (7-15) | 14 | (11-19) |
| Odds Ratio (95% CI); | 0.7 (0.4-1.2); p = 0.18 | | | |
| p-value | | | | |
| Duration of | | | | |
| Response (DOR) | | | | |
| Median in Months | 4.6 | (0.03+ to 17.6) | 4.2 | (0.03+ to 21.0) |
| (Range) | | | | |
| Progression Free | | | | |
| Survival (PFS) | | | | |
| Number of Patients | 200 | (66.7%) | 190 | (64.0%) |
| with Events (%) | | | | |
| Median in | 3.9 | (2.8 to 4.4) | 4.1 | (3.3 to 4.6) |
| Months (95% CI) | | | | |

| | |
|---|---|
| HR (95% CI); p-value | 1.00 (0.82-1.22); p = 0.990 |

These results in median OS are unexpectedly different from the Phase II data described above in Example XIII and the assumed mOS used for study powering, the latter of which were 9.1 months mOS for bavituximab plus docetaxel vs. 7.0 months mOS for docetaxel alone (473 OS events to provide 80% power and 1-sided 2.5% level of significance, assuming 9.1 vs 7.0 months mOS; HR 0.77).

Retrospective VeriStrat® proteomic testing demonstrated a VS Good signature in 80% of the bavituximab plus docetaxel group and 84% of the docetaxel alone group (Example XV). Although this Phase III trial in patients with previously treated non-squamous NSCLC did not meet the primary objective of superior OS in the bavituximab plus docetaxel arm, this outcome may be impacted by the higher than expected proportion of VS Good signature overall, and particularly in the docetaxel alone group.

### Example XV

### Initial Biomarker Analyses of the Bavituximab Phase III Trial

In connection with the Phase III trial described above, biomarker analyses were conducted with a view to identifying one or more biomarkers, or a pattern of biomarkers (a bavituximab “signature”), for patients who receive the most benefit from a bavituximab-containing therapeutic regimen. The present example concerns the sample collection techniques that apply to later studies and describes the initial proteomic signature analyses.

### A. Sample Collection

The Phase III trial was designed, and informed consent was obtained, for the collection of patient blood samples. Patient blood specimens were obtained using proper phlebotomy techniques. A tourniquet was placed 7 to 10 cm above the venipuncture site, but tourniquet application for preliminary vein selection was not permitted to exceed one minute. The patients were requested to close, but not pump, their first and the venipuncture site was cleaned with a 70% isopropyl alcohol pad using a circular motion from the center to the periphery and allow to air dry.

Using a 21 gauge needle, patient blood was collected in a 5.0 ml gold top Serum Separator Tube (SST). The tourniquet was released as soon as possible after the blood began to flow and the tube permitted to fill completely. The tube was immediately inverted 5 times after collection and allowed to clot for at least 30 minutes. To separate the serum, the tube was centrifuged within 30 to 60 minutes of collection at 1,000 to 1,300 g for 15 minutes. A pipette was used to transfer approximately 1.25 ml of serum into 3.6 ml cryovial tubes x2 and those samples were frozen.

The frozen vials were placed into a specimen bag and sealed tightly. The bottom of a dry ice shipper was layered with dry ice and the specimen bag placed in the box. Dry ice was added until the box was full, the lid was secured in place, and the samples were shipped to Central Lab for storage at −70° degrees Celsius.

The Central Lab prepared the vials for sub-aliquotting by thawing the samples. Using a pipette at least 250 μl of serum was transferred into 2 ml natural cap cryovial tubes ×4 and refrozen at −70° Celsius. Repeating the same shipping directions, the sub-aliquoted samples were shipped frozen on dry ice to the testing labs for biomarker for testing.

### B. VeriStrat® Analyses

Understanding the multi-dimensional characteristics of cancer is important to patient selection and treatment planning. The VeriStrat® test is a commercially available, blood-based predictive and prognostic proteomic test for patients with advanced NSCLC. In addition to being prognostic, VeriStrat is predictive of differential treatment benefit when selecting between single-agent treatment options. VeriStrat was retrospectively performed on patient samples from the Phase III trial.

Pre-treatment serum samples from patients in the Phase III trial were tested for protein expression using mass spectrometry, classifying patients as VeriStrat (VS) Poor (VS-P), which correlates with a more aggressive disease, or VS Good (VS-G), which correlates with a more favorable prognosis. OS was analyzed by VeriStrat subgroups using Kaplan-Meier statistical methods.

VeriStrat classification was available for 569 patients of the 597 randomized patients. In the bavituximab plus docetaxel group, 80% were VS Good and 20% were VS Poor. In the docetaxel alone group, 84% were VS Good and 16% were VS Poor. The VeriStrat Good/Poor signature was thus largely balanced between the treatment groups in the Phase III trial.

The median overall survival (mOS) in all VS Good is 11.5 months (95% confidence interval [CI], 10.6-12.9) and 5.7 (95% CI, 4.2-7.2) in all VS Poor; p<0.0001. HR OS (VS-G vs. VS-P) 0.49 (95% CI 0.37-0.64); p<0.001. These VeriStrat results are consistent with PROSE Trial (Gregorc et al., 2014) and are overall prognostic for PFS and OS.

Among VS Good patients, mOS of the bavituximab plus docetaxel arm is 11.2 months (95% CI, 10.2-12.8) and 11.8 months (95% CI, 10.4-13.5) in the docetaxel alone group; p=0.38. Among VS Poor patients, mOS of the bavituximab plus docetaxel arm is 5.8 months (95% CI, 5.0-11.3) and 4.7 months (95% CI, 3.4-7.2) in the docetaxel alone group; p=0.27. The ability of bavituximab to improve OS in VS Poor patients is important, given the limited treatment options for this group of patients.

In conclusion, the VeriStrat results in the Phase III trial are overall prognostic for PFS and OS, but not predictive for bavituximab treatment response. The unexpected OS result in the docetaxel arm may have been impacted by the relatively high overall proportion of VeriStrat Good patients. In particular, the percentage of VeriStrat Good patients in this Phase III trial (greater than 80%) is higher than previously reported (approximately 67%), indicating that patients had better prognosis overall, thus partially explaining the better than expected performance of the docetaxel arm.

Aside from the foregoing Veristrat analyses, separate proteomic approaches were also explored specifically for bavituximab. Although extensive mass spectrometry and correlative analyses were conducted to investigate possible tests able to identify a subgroup of patients benefitting from the addition of bavituximab, such gene set enrichment analyses did not result in the identification of any markers associated with clinical benefit, so emphasizing the need for further work and likely new approaches.

### Example XVI

### Assay for Functional β2GPI

The present example concerns the development of a β2GPI assay explicitly designed for the detection of functional (active) β2GPI in fluid samples. This test method is uniquely adapted to detect and quantify functional β2GPI, meaning β2GPI that is able to bind to both PS and to bavituximab. The present example thus provides a previously unavailable tool required for further meaningful biomarker analyses in connection with bavituximab treatment.

### A. Materials and Methods

### 1. Materials and Equipment

The following particular materials and equipment were used in the assay to generate the Results presented in this example under Sections B1 and B2. Materials: 96-well medium binding flat bottom plates (Greiner BioOne, cat #655001); 96-well non-binding round bottom plates (Costar, cat #3605); hexane (Sigma, cat #32293); PS antigen (Sigma, cat #P6641); ovalbumin (Sigma, cat #A5503); chromogenic substrate, tetramethylbenzidine (TMB), (KPL, cat #50-76-00); 2M H₂SO₄ (Fisher, Cat #SA818-4); plate covers (Fisher 015-027-11); adhesive plate sealer (VWR 232701); reagent reservoirs (VistaLab Cat #3054-1000). 1.5 ml microcentrifuge tubes, 50 ml conical tubes and 15 ml conical tubes were also utilized.

Equipment: vortex (Scientific Industries); timer (VWR 62344-64); pipettors from 10 to 1,000 μl (Rainin); multichannel pipettors from 100 to 300 μl (Rainin); plate reader at 450 and 650 nm (EN1835). A scale, stir bar and 37° C. incubator were also utilized. The SoftMax® Pro Software was used with the assay.

### 2. Buffers and Techniques

The Wash Buffer is 1× phosphate-buffered saline (PBS) and the Blocking Buffer is 2% Ovalbumin in 1× PBS.

Throughout the assay, subtractive pipetting was utilized when working with large volumes (e.g., ≥500 μl). The full amount of diluent was first pipetted. An equivalent volume of diluent was removed prior to adding additional reagents. All potentially hazardous vapors were handled in a fume hood.

### 3. Bavituximab-HRP

The bavituximab antibody was conjugated to horseradish peroxidase (HRP) to prepare a bavituximab-HRP detection agent for use in the assay. The conjugation was performed using EZ-Link® Plus Activated Peroxidase (Thermo Scientific, Cat #31487) following the procedure for conjugating activated peroxidase to an antibody at pH 7.2 provided by the manufacturer. Briefly, 1 mg of bavituximab was diluted to 1 mg/ml in PBS, pH 7.2. This was added to 1 mg of lyophilized EZ-Link Plus activated peroxidase to reconstitute. Immediately following reconstitution, 10 μl of 5M sodium cyanoborohydride solution was added to the reaction and incubated for 1 hour at room temperature. Once incubation was completed, 20 μl of quenching buffer was added and incubated for 15 minutes at room temperature. Conjugated bavituximab-HRP (1 mg/ml) was stored at 4° C. for up to 4 weeks.

### 4. Coating

The ELISA plates were coated with the PS antigen as follows: 5 μg/ml PS antigen was prepared and diluted into 6 ml of hexane in a fume hood with the blower off. 50 μl of PS solution was added to each well using a 12-channel pipette. The fume hood blower was turned back on and the hexane allowed to evaporate for 30-45 minutes, typically 30 minutes.

### 5. Blocking

The PS-coated ELISA plates were blocked as follows: 100 ml per plate of the Blocking Buffer (2% Ovalbumin in 1× PBS) was prepared. 200 μl of Blocking Buffer was added to each well using a 12-channel pipette. The blocked ELISA plates were incubated at 37° C. for 120 minutes (±10 minutes, which did not alter the performance of the assay).

### 6. Sample Preparations

The standard, positive control and sample preparations for the assay were performed as described below.

The β2GPI standards for the positive control were obtained from Haematologic Technologies, Inc. (HTI; cat #B2G1-0001-C; 1.0 mg/ml) in a buffer of 0.2 M Glycine, 0.15 M NaCl, pH 7.4. A vial of β2GPI was thawed and the standard and positive control preparation was performed as follows:
1 ml of β2GPI Substock A at 10 μg/ml was prepared in Blocking Buffer;
1 ml of β2GPI Substock B at 1,000 ng/ml was prepared in Blocking Buffer by subtractive pipetting 100 μl from Substock A;
1 ml of β2GPI standard at 250 ng/ml was prepared in Blocking Buffer by subtractive pipetting 250 μl from Substock B; and
control samples at 200 ng/ml, 75 ng/ml, 30 ng/ml and 5 ng/ml were prepared from the 1000 ng/ml substock according to Table 9 using subtractive pipetting.

TABLE 9
Positive Control Preparation for Functional β2GPI Assay

| Positive Control | Substock B | Blocking Buffer | Total |
|---|---|---|---|
| Concentration ng/ml | Volume (μl) | Diluent Volume (μl) | Volume (μl) |
| 200 | 200 | 800 | 1000 |
| 75 | 75 | 925 | 1000 |
| 30 | 30 | 970 | 1000 |
| 5 | 5 | 995 | 1000 |

The unknown samples were prepared for testing as follows: unknown samples were prepared in Blocking Buffer with a final dilution of 1:4000 and 1:8000; a 1:100 dilution of the unknown sample was prepared first; a 1:40 dilution was prepared from the 1:100 dilution to achieve a 1:4000 dilution; and a 1:80 dilution was prepared from the 1:100 dilution to achieve a 1:8000 dilution.

The non-binding plate preparation was performed as follows: 75 μl of Blocking Buffer was added to columns 1-3 of rows B-H; 150 μl of 250 ng/ml standard was added to columns 1-3, row A; using a multichannel pipette, 75 μl from columns 1-3 was serially diluted from row A through row G; 75 μl of positive controls and samples was added to the designated wells; and 75 μl of Blocking Buffer was added to any blank wells. The plate setup is shown in Table 10.

TABLE 10
Plate Setup for Functional β2GPI Assay

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | | |
|---|---|---|---|---|
| A | 250 ng/ml STD | (+) Control 200 ng/ml | Sample 3 Dilution 1 | Sample 7 Dilution 1 |
| B | 125 ng/ml STD | (+) Control 75 ng/ml | Sample 3 Dilution 2 | Sample 7 Dilution 2 |
| C | 62.5 ng/ml STD | (+) Control 30 ng/ml | Sample 4 Dilution 1 | Sample 8 Dilution 1 |
| D | 31.3 ng/ml STD | (+) Control 5 ng/ml | Sample 4 Dilution 2 | Sample 8 Dilution 2 |
| E | 15.6 ng/ml STD | Sample 1 Dilution 1 | Sample 5 Dilution 1 | Sample 9 Dilution 1 |
| F | 7.8 ng/ml STD | Sample 1 Dilution 2 | Sample 5 Dilution 2 | Sample 9 Dilution 2 |
| G | 3.9 ng/ml STD | Sample 2 Dilution 1 | Sample 6 Dilution 1 | Sample 10 Dilution 1 |
| H | 0 ng/ml STD | Sample 2 Dilution 2 | Sample 6 Dilution 2 | Sample 10 Dilution 2 |

### 7. Detection

Prior to finish of the block, 6 ml of 300 ng/ml bavituximab-HRP was prepared in Blocking Buffer. The assay plate was washed with 1× PBS by pipetting 250 μl into each well and this was repeated 2 more times. A plate washer may be used, in which case, the plate is washed once with 1× PBS. It was ensured that the plate was as dry as possible.

50 μl of 300 ng/ml bavituximab-HRP was added to all wells of the assay plate. 50 μl was added from each corresponding well of the non-binding plate. Using an assay plate and a non-binding plate in this way means that the detectably-labeled bavituximab is added to the PS-coated assay plate first, prior to adding the samples containing β2GPI from the non-binding plate. This sequence avoids cross-contamination during pipetting. Bavituximab-HRP and the samples containing β2GPI are incubated together on the plate and incubation was conducted at 37° C. for 90 minutes.

### 8. Development

The TMB peroxidase substrate and TMB peroxidase Solution B was removed from the refrigerator at least 1 hour before use. The assay plate was washed with 1× PBS by pipetting 250 μl into each well and this was repeated 2 more times. A plate washer may be used, in which case, the plate is washed once with 1× PBS. It was ensured that the plate was as dry as possible.

12 ml of TMB mixture was prepared by mixing 6 ml of TMB peroxidase substrate with 6 ml of TMB solution B. 100 μl of TMB solution was added to each well of the assay plate and allowed to develop for 5-6 minutes. Development was stopped by adding 100 μl of 2M H₂SO₄ to each well of the assay plate. The assay plate was read and optical density (OD) determined at 450nm within 30 minutes of stopping the reaction. The microplate reader was used in conjunction with the SoftMaxPro plate data and analysis template, which provides a printout of assay data.

### 9. Preparation of Nicked β2GPI

Samples of β2GPI purified from human plasma and recombinant human β2GPI were both treated with plasmin (enzyme hydrolysis) to prepare samples that contained a majority of nicked β2GPI. The nicked β2GPI was not purified to homogeneity for initial studies, but nicked β2GPI was determined to be present in excess over the non-nicked, or “intact” β2GPI.

### 10. Assay for Total β2GPI

An assay was designed that should detect total β2GPI, based on the manufacturer's specifications for the antibodies used. This is an assay using commercially available antibodies from US biological, in which plates are coated with a capture antibody against β2GPI and any bound β2GPI is detected using an anti-β2GPI-HRP conjugate as a detection antibody. The antibody catalog numbers are: Capture Antibody, US Biological #A2299-81A, affinity-purified anti-β2GPI and Detecting Antibody, US Biological #A2299-81B, peroxidase-conjugated anti-β2GPI.

A 1:100 dilution of the capture antibody was prepared in carbonate buffer (50 mM Sodium Bicarbonate) at pH 9.6. 100 μl was added to each well of the ELISA plate and incubated at room temperature. The plate was washed with 1× PBS buffer containing Tween-20, then blocked with 200 μl/well of assay diluent containing 1% BSA and incubated at 37° C. Purified β2GPI was used to prepare a two-fold dilution standard curve starting at 500 ng/ml in assay diluent. Samples were diluted in assay diluent to achieve a concentration within the linear region of the standard curve. After the blocking incubation, the plate was washed, followed by the addition of 100 μl/well of the standard curve and samples in either duplicate or triplicate. After the addition of the standard curve and samples, the plate was incubated at 37° C. The detection antibody was diluted 1:400 in assay diluent. After incubating the samples and standard curve, the plate was washed, followed by the addition of 100 μl/well of the detection antibody. The plate was incubated at 37° C. After the secondary antibody incubation, the plate was washed, then developed with TMB. The plate was read on a plate reader at 450nm and the sample concentrations determined from the standard curve.

### B. Results

1. Distinguishing Functional from Nicked β2GPI

β2GPI purified from human plasma (“human”) or following recombinant expression (“recombinant”) was treated with plasmin to prepare β2GPI test samples that contained a majority of plasmin-cleaved (nicked) β2GPI, which does not bind to PS. Those samples were tested alongside plasmin-free (intact) β2GPI, and a 50:50 mixture of each, in the present assay (Table 11B) and using an assay designed to detect total β2GPI using commercially available capture and detection antibodies (Table 11A). The results are shown below.

TABLE 11A
Testing Nicked and Functional β2GPI in Total β2GPI Assay

| Sample | | Conc. (ng/ml) |
|---|---|---|
| Human | Plasmin-treated β2GPI | 104.12 |
| | 50:50 Mix | 119.77 |
| | Plasmin-free β2GPI | 140.90 |
| Recombinant | Plasmin-treated β2GPI | 141.35 |
| | 50:50 Mix | 134.51 |
| | Plasmin-free β2GPI | 140.90 |

TABLE 11B
Testing Nicked and Functional β2GPI in Functional β2GPI Assay

| Sample | | Conc. (ng/ml) |
|---|---|---|
| Human | Plasmin-treated β2GPI | 32.95 |
| | 50:50 Mix | 80.86 |
| | Plasmin-free β2GPI | 136.42 |
| Recombinant | Plasmin-treated β2GPI | 33.26 |
| | 50:50 Mix | 88.29 |
| | Plasmin-free β2GPI | 136.42 |

It can first be seen that the so-called “total β2GPI assay” using commercially available antibodies (Table 11A), and the present, “functional β2GPI assay” (Table 11B), both read out similar concentrations of β2GPI (approximately 141 ng/ml and 136 ng/ml). Using the total β2GPI assay, there is essentially no difference in detecting plasmin-treated recombinant β2GPI, and only a moderate reduction in detection as the amounts of plasmin-treated β2GPI from human plasma are increased (141 to 104 ng/ml). In contrast, using the functional β2GPI assay, increasing amounts of plasmin-treated β2GPI, either recombinant or plasma-derived, result in a significant reduction in binding (136 to 33 ng/ml).

Consistent with the design of the assay, these results therefore show that the present assay is able to effectively detect functional β2GPI, i.e., β2GPI that binds to both PS and to bavituximab, as opposed to nicked β2GPI. This distinguishes the present, functional β2GPI assay from commercially available assay kits (and assays using commercially available anti-β2GPI antibodies), which detect nicked β2GPI (non PS-binding) along with β2GPI that does bind to PS.

### 2. Quantifying Functional β2GPI

The assay is able to successfully determine the amount of functional β2GPI in fluid samples, which is β2GPI that binds to both PS and to bavituximab. This assay has now been routinely performed to prepare reproducible β2GPI standard curves. In this regard, a Four-Parameter Logistic Fit is used, which is a statistical equation used for non-linear regression analysis. The Four-Parameter Fit Equation is:

$$y = \frac{( A - D )}{( 1 + {( \frac{x}{C} )}^{B} )} + D$$

Where:
A is the Y-value corresponding to the asymptote (i.e. the flat part of the curve) for the low values of the X-axis;
B is the coefficient that describes how rapidly the curve makes its transition from asymptotes in the center of the curve, and is commonly called as the slope factor;
C is the X value corresponding to the midpoint between A and D; commonly called the EC50; and
D is the Y-value corresponding to the asymptote for the high values of the X-axis.

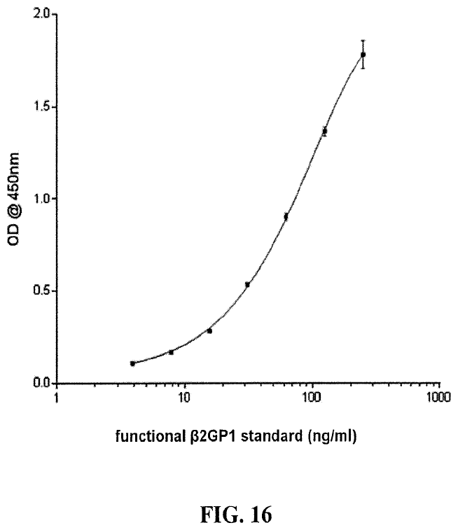

A representative example of a standard curve for functional β2GPI is shown in FIG. 16. From such a standard curve, the concentrations of functional β2GPI in human blood samples, such as plasma or serum samples, can be determined. Mainly for accuracy, but also for economy of sample preparation, the standard curve is prepared in ng/ml (nanogram/ml). As the average levels of β2GPI in the normal human population are about 200 μg/ml (microgram/ml) (Mehdi et al., 1999; Miyakis et al., 2004), the standard curve is prepared in expectation that the test samples will be diluted before analysis in the assay. Diluted plasma or serum test samples are run in the assay and the concentration of β2GPI in the patient then calculated by adjusting for the dilution factor.

This assay has now been used to determine the levels of functional β2GPI in the patients from the above Phase III trial, the results of which are presented in Example XVII, below, and in Example XVIII and Example XX.

### 3. Alternative, Equivalent Assay Components and Steps

In addition to the particular materials, equipment and assay steps described in this example under Sections A1-A8, variations in the components and method steps can be made and executed without departing from the concept of the assay to detect and quantify functional β2GPI. The following results show that related agents may be substituted for the agents described in Sections A1-A8 and essentially the same results achieved.

Certain preferred ELISA plates are those optimized for lipid adsorption, which may be used to replace the ELISA plates in Section A1, above. ELISA plates are known that are optimized for lipid adsorption, which have surface chemistries providing better lipid (PS) binding. One such ELISA plate is the ThermoFisher PolySorp® plate, which has been used in a new assay format.

The hexane-based PS coating method in Section A4, above, may preferably be replaced with an isopropanol-based PS coating method, which can provide certain safety benefits to the user (by avoiding the use of hexane). In using isopropanol as a coating buffer in a new assay format, the ELISA plates are coated with PS antigen using 10 μg/ml PS antigen diluted in isopropanol and the incubation time is 90 min.

To produce an effective β2GPI calibration curve, any known method of obtaining β2GPI may be employed. For example, as purchased from a commercial vendor, such as HTI (Section A6, above). Alternative β2GPI preparations may also be developed for defined, reproducible calibration control. One such preferred method is to express β2GPI in CHO cells and purify the expressed β2GPI.

A preferred purification of β2GPI from CHO cells includes: a harvest clarification, chromatin extraction step, which removes contaminants and allows the clarified harvest to pass through a 0.2 μm filter; use of a tangential flow filtration (TFF) system, to buffer-exchange the clarified harvest and decrease its conductivity without increasing the volume; a capto adhere step in anion flow through mode, to remove further contaminants; a strong cation step using Nuvia™ S to remove aggregates and other contaminants, concentrate the eluate and facilitate any buffer exchange step; and, optionally, use of a TFF system to buffer exchange and concentrate the purified β2GPI. β2GPI has been expressed and purified in this way and used in a new assay format.

In addition to Section A3, above, certain preferred bavituximab-HRP detection agents are conjugates crosslinked using either of two commonly-used, non-proprietary crosslinkers, SMCC (succinimidyl 4-(N-maleimidomethyl)cyclohexane-1-carboxylate) or SATA (N-succinimidyl S-acetylthioacetate). Other preferred bavituximab-HRP detection agents are conjugates in which the number of HRP is in excess to the bavituximab antibody, particularly those resulting in an HRP:bavituximab ratio of 2:1 or 3:1, with essentially no free (unconjugated) antibody. Such conjugates are purified by an S-300 sizing column to remove unreacted reaction components. Bavituximab-HRP detection agents with each of these constituents and properties have been obtained from Columbia Biosciences, 4985 Winchester Blvd., Frederick, Md., 21703, and used at 600 ng/ml in a new assay format.

Whilst one or more of the above alternative components and assay steps may be preferred, particularly for technical reasons, even the combined use of all such alternatives provides a functional β2GPI assay that gives essentially the same results as the assay originally described in this example, i.e., under Sections A1-A8. Such comparative results are shown below in Table 12, which presents the functional β2GPI levels measured in the two different assay formats using four random human samples (Donor) obtained from the San Diego Blood Bank.

TABLE 12
Comparable Performance of Functional β2GPI Assays

| β2GPI Concentration (μg/ml) | |
|---|---|
| Assay Format, | Assay Format, |
| Example XVI, A1-A8 | Example XVI, B9 |

| Donor | Mean | SD | Mean | SD |
|---|---|---|---|---|
| 1 | 214 | 21 | 214 | 20 |
| 2 | 276 | 20 | 281 | 33 |
| 3 | 224 | 22 | 226 | 22 |
| 4 | 223 | 21 | 219 | 23 |

### Example XVII

### β2GPI Biomarker Analyses in the Bavituximab Phase III Trial

Utilizing the functional β2GPI assay described above, the present example reports the levels of pre-treatment functional β2GPI in the patients of the Phase III trial of Example XIV. By correlating the levels of functional β2GPI with treatment outcomes, the present example also concerns functional β2GPI as a biomarker for successful bavituximab treatment, such as in NSCLC patients treated with bavituximab and docetaxel and other combination therapies.

### A. Functional β2GPI Levels in Patients

The Phase III trial described above accrued 597 patients. The collection of blood samples from the patients in the Phase III trial is described in Example XV, A. At the time of the present analyses, there were 592 patient samples evaluable for functional β2GPI. Sub-aliquots of those 592 patient blood samples were tested for functional β2GPI, using the assay described in the example immediately above.

The levels of pre-treatment functional β2GPI in μg/ml and a summary of the statistics are presented in Table 13, in which the ‘Bavituximab’ row refers to patients treated with bavituximab plus docetaxel and the ‘Placebo’ row refers to patients treated with docetaxel alone.

TABLE 13
Functional β2GPI Levels in Phase III Patients

| | N | Mean (SD) | Min | Q1 | Median | Q3 | Max |
|---|---|---|---|---|---|---|---|
| Bavituximab | 294 | 202 (57.3) | 22 | 162 | 207 | 240 | 365 |
| Placebo | 298 | 195 (59.7) | 0.5 | 159 | 199 | 238 | 402 |
| All | 592 | 198 (58.6) | 0.5 | 161 | 203 | 239 | 402 |

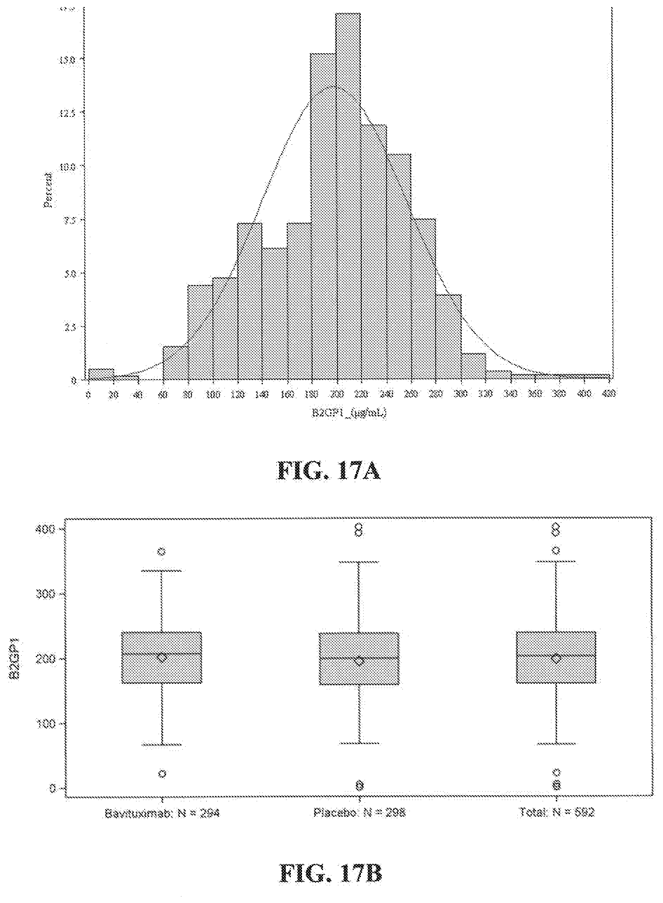

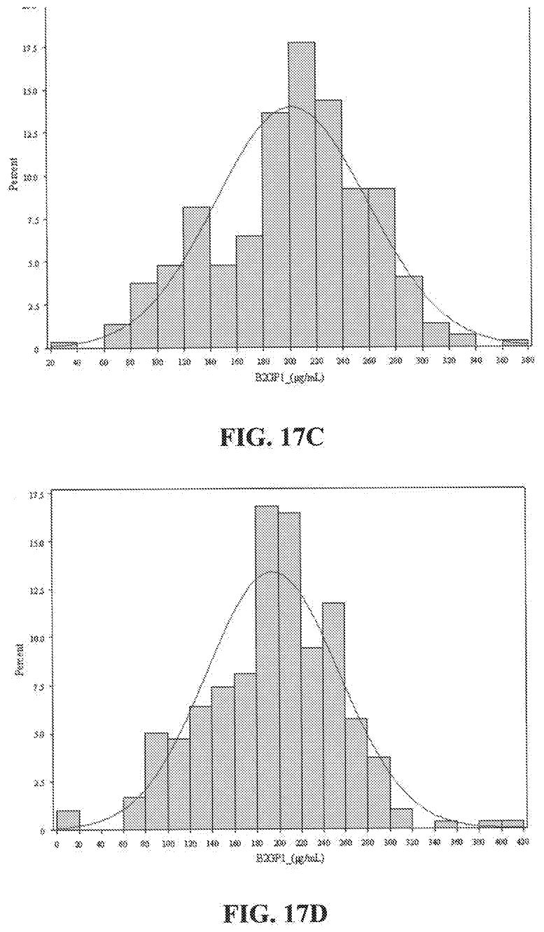

The levels of pre-treatment functional β2GPI ranged from 0.5 to 402 μg/ml and the distribution of functional β2GPI for all patients is shown in FIG. 17A and FIG. 17B. Within the patients treated with bavituximab plus docetaxel, functional β2GPI ranged from 22 to 365 μg/ml, as shown FIG. 17C. The distribution of functional β2GPI in the patients treated with docetaxel alone is shown in FIG. 17D, which covers the full range for the study (0.5 to 402 μg/ml).

For each treatment group (202 and 195 μg/ml), and for the study overall (198 μg/ml), the levels of pre-treatment functional β2GPI are consistent with the average of 200 μm/ml reported in the literature (20 mg/dl by Mehdi et al., 1999 and 200 mg/l by Miyakis et al., 2004).

It was determined that the percentage of patients with levels of pre-treatment functional β2GPI of equal to or greater than 200 μg/ml was 56% for patients treated with bavituximab plus docetaxel, and 49% for patients treated with docetaxel alone.

### B. Single Cutoff β2GPI Biomarker Analyses

Sub-group analyses performed to evaluate functional β2GPI as a predictor of response in patients receiving bavituximab plus docetaxel therapy demonstrated strong trends for prolonged survival.

A single cutoff method was first used to assess the patient β2GPI data. In searching for the optimal cutoff in this manner, step 1 is to search for OS separation or significant OS separation of a High β2GPI vs. Low β2GPI group for patients in the bavituximab plus docetaxel group; step 2 is to search for OS separation or significant OS separation of the bavituximab plus docetaxel group vs. the docetaxel alone group (placebo) for those High β2GPI patients.

Initial analyses of functional β2GPI as a possible biomarker by applying the single cutoff method to 578 evaluable patients surprisingly indicated that in patients with high β2GPI, the mOS was 11.9 months (95% CI, 9.0-14.7) among 167 patients in the bavituximab plus docetaxel group and 9.4 months (95% CI, 7.7-11.7) among 141 patients in the docetaxel alone group (HR for death, 0.77; P=0.1). In these initial analyses, “high β2GPI” is defined as pre-treatment levels of functional β2GPI of equal to or higher than 200 μg/ml (≥200 μg/ml). As these analyses are based on a single cutoff, patients not having “high β2GPI” have functional β2GPI of less than 200 μg/ml (<200 μg/ml).

The single cutoff analyses were then extended to the 592 evaluable patients. Although not statistically significant, these analyses also demonstrated a surprising trend for prolonged survival in the bavituximab plus docetaxel group when patients had pre-treatment levels of functional β2GPI of equal to or greater than 200 μg/ml. These results are represented by the Kaplan-Meier survival curves for functional β2GPI of ≥200 μg/ml in FIG. 18A and FIG. 18B. Of the 592 evaluable patients, FIG. 18A shows that for patients treated with bavituximab, those with functional β2GPI of equal to or higher than 200 μg/ml (167 patients) had a mOS of 11.4 months, vs. only 9.8 months for 127 patients with “low β2GPI” of less than 200 μg/ml (HR for death, 0.76; P=0.054 with CI (0.58, 1.01)). As shown in FIG. 18B, in patients with pre-treatment levels of functional β2GPI≥200 μg/ml, representing approximately 52% of randomized patients, the mOS was 11.4 months (95% CI,8.4-16.6) among 167 patients in the bavituximab plus docetaxel group and 10.2 months (95% CI, 8.5-11.9) among 146 patients in the docetaxel alone group (HR for death, 0.82; P=0.134 with CI (0.63, 1.06)).

### C. Two Cutoff β2GPI Biomarker Analyses

The single cutoff analyses described above were followed by further analyses of the data in 592 evaluable patients using a two cutoff method (Klein & Moeschberger, 2003). In the two cutoff method, Step 1 is to search for significant OS separation of “Within Range” vs. “Outside of Range” for patients treated with bavituximab (plus docetaxel), and Step 2 is to search for significant OS separation of the bavituximab vs. placebo arms for patients “Within Range”.

These sub-group analyses using the two cutoff method in 592 evaluable patients produced a number of statistically significant ranges of functional β2GPI showing a survival benefit for bavituximab, each starting with functional β2GPI at 200 μg/ml or above, thus validating the initial surprising finding that pre-treatment levels of functional β2GPI of equal to or greater than 200 μg/ml are beneficial for treatment with bavituximab. In particular, the two cutoff method showed that pre-treatment levels of functional β2GPI within each of the ranges of 210-270, 210-280, 210-290, 200-280 and 200-290 μg/ml are statistically significant predictors of benefit in overall survival in patients treated with bavituximab plus docetaxel vs. those treated with docetaxel alone. These results for the functional β2GPI ranges of 210-270, 210-280, 210-290, 200-280 and 200-290 μg/ml are shown in Table 14A and Table 14B.

TABLE 14A
Ranges of Pre-Treatment β2GPI
Improve Bavituximab Overall Survival

| | | In Range | | | | | |
|---|---|---|---|---|---|---|---|
| β2GPI | | Patients (N) | | | In Range | | |
| (μg/ml) | | | Pla- | | Hazard | | |
| Low | High | Bavi | cebo | Total | Ratio | CI | P value |
| 210 | 280 | 124 | 102 | 226 | 0.689891086 | 0.51, 0.94 | 0.0184092 |
| 210 | 290 | 131 | 108 | 239 | 0.702335779 | 0.52, 0.95 | 0.0206955 |
| 210 | 270 | 111 | 93 | 204 | 0.704885964 | 0.51, 0.98 | 0.0356625 |
| 200 | 280 | 149 | 129 | 278 | 0.750114990 | 0.57, 1.00 | 0.0455542 |
| 200 | 290 | 156 | 135 | 291 | 0.758545153 | 0.58, 1.00 | 0.0486627 |

TABLE 14B
Ranges of Pre-Treatment β2GPI
Improve Bavituximab Overall Survival

| | | Outside Range | | | | | |
|---|---|---|---|---|---|---|---|
| β2GPI | | Patients (N) | | | Outside Range | | |
| (μg/ml) | | | Pla- | | Hazard | | |
| Low | High | Bavi | cebo | Total | Ratio | CI | P value |
| 210 | 280 | 170 | 196 | 366 | 1.326781940 | 1.03, 1.70 | 0.0260507 |
| 210 | 290 | 163 | 190 | 353 | 1.350309796 | 1.05, 1.74 | 0.0205255 |
| 210 | 270 | 183 | 205 | 388 | 1.267025810 | 1.00, 1.61 | 0.0537042 |
| 200 | 280 | 145 | 169 | 314 | 1.366480252 | 1.05, 1.79 | 0.0217840 |
| 200 | 290 | 138 | 163 | 301 | 1.396948670 | 1.06, 1.84 | 0.0164154 |

Summarizing the results in Table 14A, each of the ranges of functional β2GPI of 210-270, 210-280, 210-290, 200-280 and 200-290 μg/ml have a hazard ratio of less than one, and a statistically significant P value, representing the improvement in survival. Table 14B naturally shows the opposite, in that patients with functional β2GPI outside of those stated ranges have a hazard ratio of more than one, and a statistically significant P value, representing a worsening of survival (or increased chance of death). For example, patients with pre-treatment β2GPI levels of 200-290 μg/ml, representing approximately 49% of randomized patients, had a mOS of 11.4 months when treated with bavituximab (plus docetaxel) vs. only 10.1 months for patients in the control group with the same range of β2GPI levels. This 11.4 month vs. 10.1 month increase reflects a statistically significant improvement in mOS (HR 0.76, P=0.049).

There is no suggestion in the literature that pre-treatment levels of functional β2GPI of equal to or greater than 200 μg/ml would indicate a trend for prolonged survival on bavituximab treatment, and no suggestion that pre-treatment levels of functional β2GPI of 210-270, 210-280, 210-290, 200-280 or 200-290 μg/ml would be predictive of benefit in overall survival in patients treated with bavituximab. Indeed, there is nothing in the significant prior clinical experience with bavituximab to suggest such outcomes. Moreover, such findings are very much at odds with the data from extensive pre-clinical modelling, which indicated that varying levels of serum β2GPI would not significantly impact treatment outcomes for bavituximab. The pre-clinical experience, in particular, rather indicated that quite low levels of serum β2GPI, such as on the order of 10-20 to 50-60 μg/ml or so, would be sufficient to support bavituximab binding and activity (Example V).

In particular, using different assays, Example V shows that molar ratios of β2GPI to antibody of 0.12 to 0.25 (FIG. 9A and FIG. 9B, with FIG. 1A, FIG. 1B, FIG. 1C and FIG. 1D); 0.125, 0.5 to 2 (FIG. 10); 0.93 (FIG. 11); and 1.43 to 2.86 (FIG. 12) are effective in supporting binding of bavituximab to PS. Considering several different binding and functional test systems, including pre-clinical data indicating that bavituximab is effective at molar ratios of β2GPI to antibody of about 2.86 (Table 3), a molar ratio of β2GPI to antibody of above 3 should not be needed. In using a dose of 3 mg/kg of bavituximab in the present Phase III trial, such ratios are achieved at β2GPI levels below 60 μg/ml (FIG. 19). For reference, the amounts of β2GPI, antibody and comparable β2GPI-antibody ratios for the Phase III trial are shown in Table 15, where N=the number of patients (from the 592 evaluable patients) having levels of functional β2GPI within each defined increment.

TABLE 15
β2GPI and Antibody Levels and Ratios in Phase III Patients

| Bavi | Cmax | Bavi | β2GPI | | β2GPI | Molar Ratio |
|---|---|---|---|---|---|---|
| (mg/kg) | (μg/ml) | (μM) | (μg/ml) | N= | (μM) | β2GPI to Ab |
| 3 | 56.4 | 0.389 | 10 | 3 | 0.2 | 0.514 |
| 3 | 56.4 | 0.389 | 20 | | 0.4 | 1.028 |
| 3 | 56.4 | 0.389 | 30 | 1 | 0.6 | 1.542 |
| 3 | 56.4 | 0.389 | 40 | | 0.8 | 2.057 |
| 3 | 56.4 | 0.389 | 50 | 0 | 1.0 | 2.571 |
| 3 | 56.4 | 0.389 | 60 | | 1.2 | 3.085 |
| 3 | 56.4 | 0.389 | 80 | 9 | 1.6 | 4.113 |
| 3 | 56.4 | 0.389 | 100 | 26 | 2.0 | 5.141 |
| 3 | 56.4 | 0.389 | 120 | 28 | 2.4 | 6.170 |
| 3 | 56.4 | 0.389 | 140 | 43 | 2.8 | 7.198 |
| 3 | 56.4 | 0.389 | 160 | 36 | 3.2 | 8.226 |
| 3 | 56.4 | 0.389 | 180 | 43 | 3.6 | 9.254 |
| 3 | 56.4 | 0.389 | 200 | 90 | 4.0 | 10.283 |
| 3 | 56.4 | 0.389 | 220 | 101 | 4.4 | 11.311 |
| 3 | 56.4 | 0.389 | 240 | 70 | 4.8 | 12.339 |
| 3 | 56.4 | 0.389 | 260 | 62 | 5.2 | 13.368 |
| 3 | 56.4 | 0.389 | 280 | 44 | 5.6 | 14.396 |
| 3 | 56.4 | 0.389 | 300 | 23 | 6.0 | 15.424 |
| 3 | 56.4 | 0.389 | 320 | 7 | 6.4 | 16.452 |
| 3 | 56.4 | 0.389 | 340 | 2 | 6.8 | 17.481 |
| 3 | 56.4 | 0.389 | 360 | 1 | 7.2 | 18.509 |
| 3 | 56.4 | 0.389 | 380 | 1 | 7.6 | 19.537 |
| 3 | 56.4 | 0.389 | 402 | 2 | 8.0 | 20.566 |

In comparing Table 15 and FIG. 17A to the data used for modelling (Table 1, Table 2 and Table 3), it can be seen that the vast majority of patients in the Phase III trial had levels of functional β2GPI that equated to β2GPI to antibody molar ratios that were more than sufficient to saturate bavituximab binding (≥2.86), i.e., starting from 60 μg/ml or 1.2 μM (Table 15; FIG. 19), even when bavituximab was at its maximum concentration in the blood (Cmax of 56.4 μg/ml; Example VI; Gerber et al., 2011). In fact, only 4 out of 592 evaluable patients (0.68%) had pre-treatment levels of functional β2GPI of less than 60 μg/ml. Moreover, as the levels of functional β2GPI increase, which was the case for the majority of patients in the trial, the molar ratios of β2GPI to bavituximab are much higher than 2 or 3, such as being over 10 at 200 μg/ml. Nothing in the prior pre-clinical modelling or clinical experience pointed towards such β2GPI levels or ratios being beneficial for bavituximab therapy. Rather, as shown in FIG. 19, pre-clinical data indicated that low levels of serum β2GPI, starting at about 10 μg/ml or even less (β2GPI at 5 μg/ml has a β2GPI:Ab molar ratio of 0.257), and comfortably at about 60 μg/ml, would be sufficient to support bavituximab binding and activity (Example V).

Although unexpected, these detailed analyses of the pre-treatment levels of functional β2GPI as a possible biomarker for bavituximab outcomes are highly encouraging. Measuring pre-treatment concentrations of functional β2GPI in patients thus provides a strategy to predict response to bavituximab therapy, i.e., to select patients who are more, and most, likely to benefit from treatment with bavituximab. This was first observed in the use of bavituximab with docetaxel, particularly in NSCLC. However, as the mechanisms of bavituximab binding in a complex with functional β2GPI and PS, and the immune activating mechanisms of bavituximab overall, are common to all bavituximab therapies, the selection of patients based on pre-treatment levels of functional β2GPI of equal to or greater than 200 μg/ml can therefore be included in all future trials and therapies using bavituximab with a well-founded expectation that this will improve the treatment outcomes. Indeed, further evidence supporting this is provided in Example XVIII and Example XX.

### Example XVIII

### β2GPI Biomarker Analyses in Further Bavituximab Clinical Trials

Following the identification of functional β2GPI as a biomarker for successful bavituximab treatment in Example XVII, the present example extends the use of the functional β2GPI assay to samples from earlier bavituximab clinical trials. The following results show that the same levels of functional β2GPI also correlate with successful treatment outcomes for bavituximab, thus confirming functional β2GPI as a biomarker for bavituximab.

### A. Phase II Trial of Example XIII

Samples from the NSCLC Phase II trial of Example XIII (PPHM 0902) were tested using the functional β2GPI assay of Example XVI. There were 119 patient samples in which levels of pre-treatment functional β2GPI were evaluable, of which 40 patients were in the bavituximab 3 mg/kg arm and 79 patients were in the combined control arm (placebo or 1 mg/kg bavituximab).

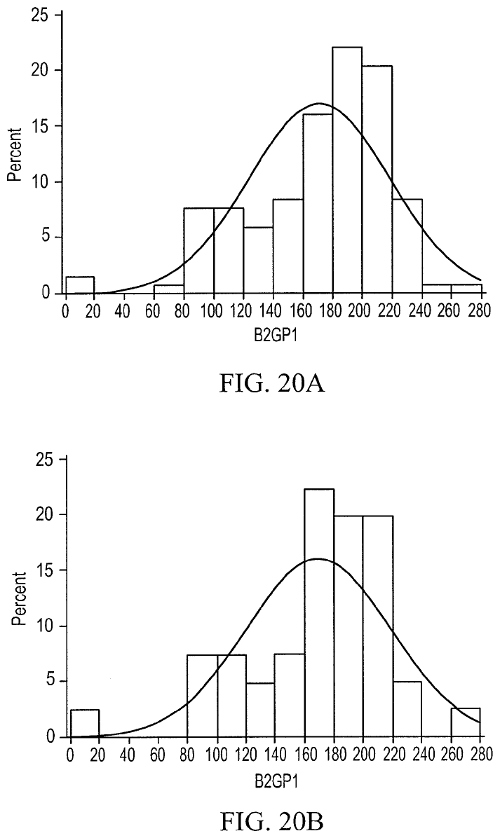

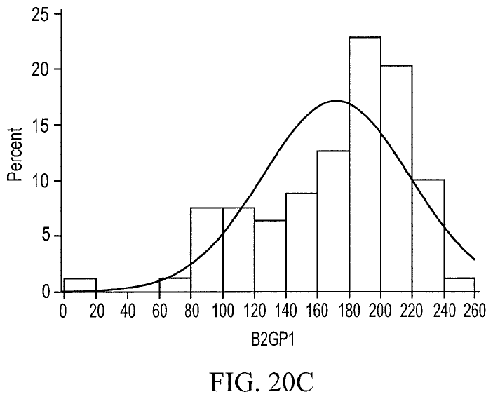

The levels of pre-treatment functional β2GPI ranged from 0.5 to 266 μg/ml for all patients (FIG. 20A). Within the patients treated with bavituximab 3 mg/kg plus docetaxel, functional β2GPI ranged from 0.5 to 266 μg/ml (FIG. 20B). The distribution of functional β2GPI in the patients in the combined control arm was 0.5 to 257.4 μg/ml (FIG. 20C). For each treatment group (169.4 μg/ml for bavituximab 3 mg/kg, and 171.8 μg/ml for combined control arm), and for the study overall (171.0 μg/ml), the levels of pre-treatment functional β2GPI are consistent with the average reported in the literature.

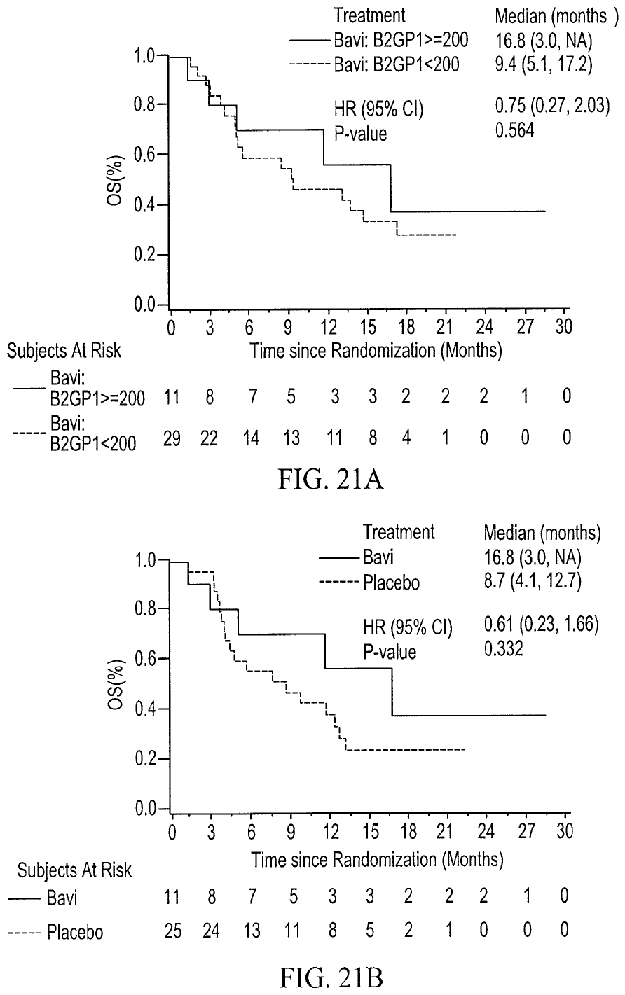

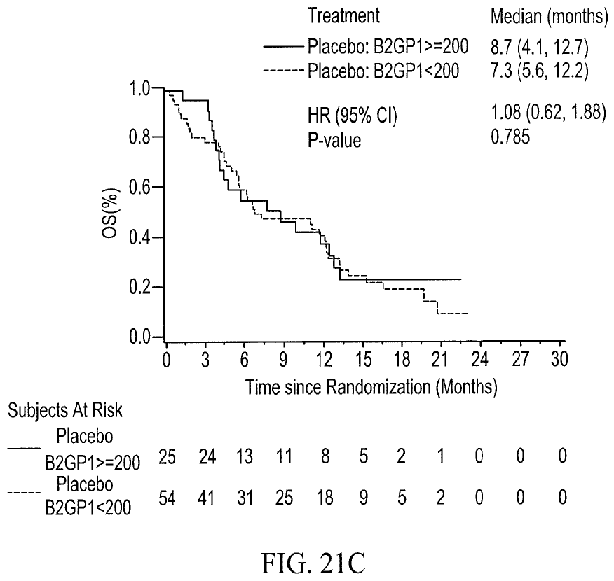

Using a cut-off of “high β2GPI” being defined as pre-treatment levels of functional β2GPI of equal to or higher than 200 μg/ml (≥200 μg/ml), it was determined that β2GPI≥200 μg/ml trended with increased overall survival in the bavituximab 3 mg/kg arm (FIG. 21A and FIG. 21B), but not in the other arm (FIG. 21C). For example, for patients treated with bavituximab 3 mg/kg, those with functional β2GPI of equal to or higher than 200 μg/ml had a mOS of 16.8 months, vs. only 9.4 months for “low β2GPI” of less than 200 μg/ml (FIG. 21A). Also, in patients with functional β2GPI≥200 μg/ml, the 16.8 months mOS for patients treated with bavituximab 3 mg/kg exceeded that of only 8.7 months mOS for patients in the combined control arm (FIG. 21B). Contrast the clear separation of the curves in each of FIG. 21A and FIG. 21B with the super-imposed curves in FIG. 21C (comparing β2GPI≥200 μg/ml to β2GPI<200 μg/ml in the combined control arm).

### B. Phase II Trial of Example XII

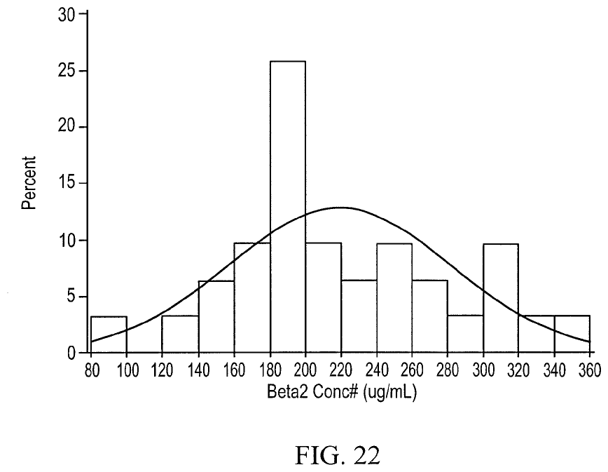

Samples from the Phase II pancreatic cancer trial of Example XII (PPHM 1002) were tested using the functional β2GPI assay of Example XVI. There were 31 patient samples in which levels of pre-treatment functional β2GPI were evaluable. The levels of pre-treatment functional β2GPI ranged from 82.5 to 343.2 μg/ml for all patients (FIG. 22). For these 31 patients, the mean level of pre-treatment functional β2GPI (219.2 μg/ml) was consistent with the average reported in the literature.

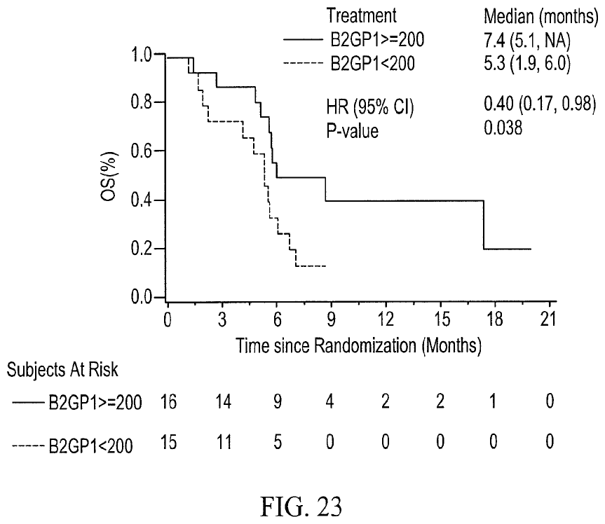

Although the sample size is small, and the disease is very aggressive, using a cut-off of “high β2GPI” of functional β2GPI of equal to or higher than 200 μg/ml (≥200 μg/ml), it was determined that β2GPI≥200 μg/ml trended with increased overall survival for bavituximab. Patients treated with bavituximab having functional β2GPI of equal to or higher than 200 μg/ml had a mOS of 7.4 months, vs. 5.3 months for “low β2GPI” of less than 200 μg/ml (FIG. 23).

### C. Phase II Trial of Bavituximab and Paclitaxel/Carboplatin in NSCLC

A randomized, open-label, Phase II trial (PPHM 1001) of paclitaxel/carboplatin with or without bavituximab was conducted in patients with previously untreated locally advanced or metastatic non-squamous NSCLC. Samples from this trial were tested using the functional β2GPI assay of Example XVI. There were 84 patient samples in which levels of pre-treatment functional β2GPI were evaluable, of which 44 patients were in the bavituximab arm and 40 patients were in the paclitaxel/carboplatin arm.

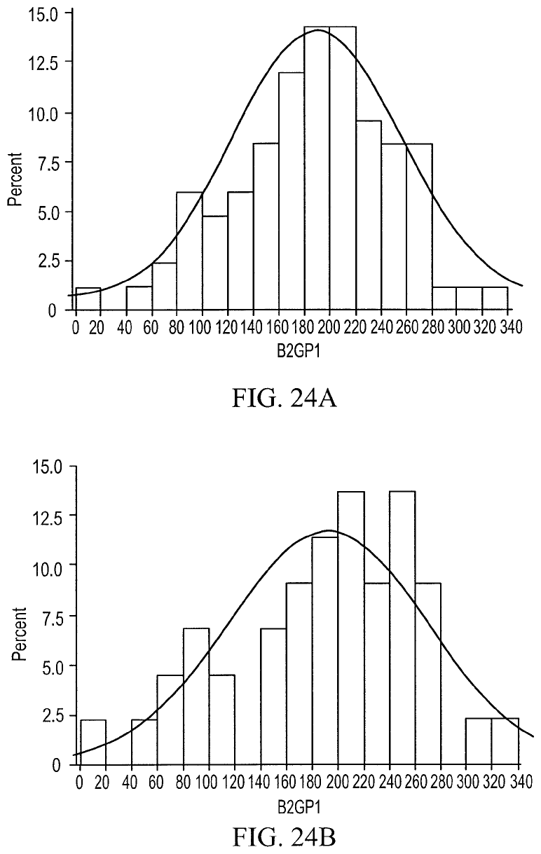

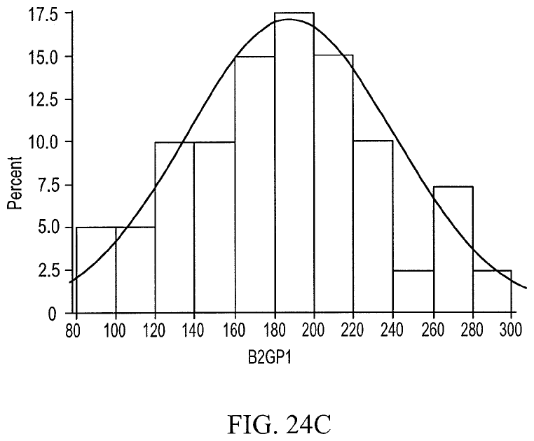

The levels of pre-treatment functional β2GPI ranged from 0.5 to 326 μg/ml for all patients (FIG. 24A). Within the patients treated with bavituximab, functional β2GPI ranged from 0.5 to 326 μg/ml (FIG. 24B). Functional β2GPI in the patients in the paclitaxel/carboplatin arm ranged from 88.8 to 292.7 μg/ml (FIG. 24C). For each treatment group (187.9 μg/ml for bavituximab, and 186.4 μg/ml for the paclitaxel/carboplatin arm), and for the study overall (187.2 μg/ml), the levels of pre-treatment functional β2GPI are again consistent with the average reported in the literature.

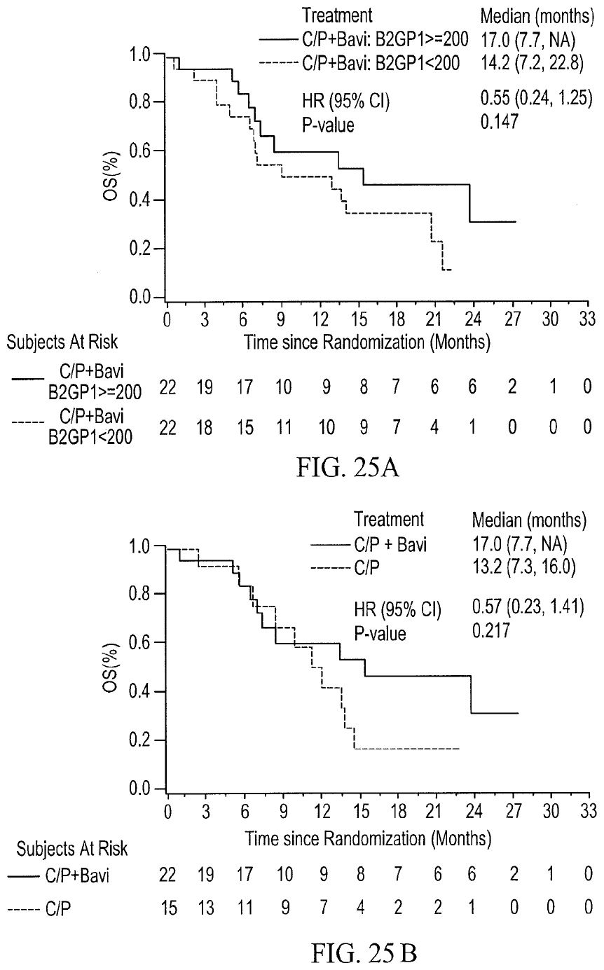

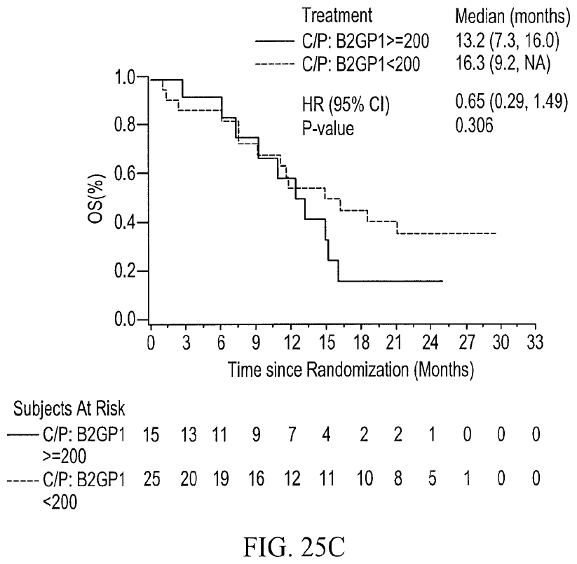

Using the same cut-off of “high β2GPI” as being pre-treatment levels of functional β2GPI of equal to or higher than 200 μg/ml (≥200 μg/ml), it was determined that β2GPI≥200 μg/ml again trended with increased overall survival in the bavituximab arm, but not in the control (paclitaxel/carboplatin) arm. For example, for patients treated with bavituximab, those with functional β2GPI of equal to or higher than 200 μg/ml had a mOS of 17.0 months, vs. 14.2 months for “low β2GPI” of less than 200 μg/ml (FIG. 25A). Also, in patients with functional β2GPI≥200 μg/ml, the 17.0 months mOS for patients treated with bavituximab exceeded that of only 13.2 months mOS for patients in the control arm (FIG. 25B). Contrast the separation of the curves in FIG. 25A and FIG. 25B, particularly FIG. 25A, with FIG. 25C, in which there is a trend for patients in the control arm to survive longer when β2GPI is less than 200 μg/ml.

In conclusion, the data in Example XVII and Example XVIII, from four separate clinical trials, consistently show that functional β2GPI levels correlate with treatment outcomes, thus validating functional β2GPI levels as a biomarker for successful bavituximab treatment.

### EXAMPLE XIX

### Survival Benefit for Bavituximab in Combination with Subsequent Immunotherapy

Although the initial analyses of the Phase III trial of Example XIV did not show superior OS in the bavituximab plus docetaxel arm as compared to the docetaxel alone group, ongoing studies were conducted with a view to identifying other possible indicators of a therapeutic benefit to bavituximab treatment. The present example shows that patients treated with bavituximab and docetaxel followed by subsequent immunotherapy (SACT-IO) have a statistically significant better mOS as opposed to patients treated with docetaxel alone followed by subsequent immunotherapy.

Following treatment with either bavituximab and docetaxel, or docetaxel alone, about 15% of the patients (93 out of 597) received subsequent anti-cancer therapy (SACT), in the form of subsequent immuno-oncology (IO) therapy (SACT-IO or subsequent IO), using an immune checkpoint inhibitors (ICI). These 93 patients were evenly balanced between the treatment arms, with 46 patients receiving prior treatment with bavituximab and docetaxel, and 47 patients receiving prior treatment with docetaxel alone.

Surprisingly, it was determined that there was a dramatic increase in mOS for patients receiving prior treatment with bavituximab, as opposed to placebo, when treated with subsequent IO (FIG. 26). In particular, for patients receiving subsequent IO, mOS has yet to be reached for the bavituximab and docetaxel group (95% CI, 15.2-NA), whereas it was 12.6 months for the docetaxel alone group (95% CI, 10.4-17.8); HR=0.46 and p=0.006 (FIG. 26; Table 16). For patients who did not receive subsequent IO, mOS was 9.2 months in the bavituximab and docetaxel group and 10.2 months in the docetaxel alone group; HR=1.16 and p=0.172.

TABLE 16
Survival Benefit for Bavituximab in Combination
with Subsequent Immunotherapy

| | | Bavituximab + | Placebo + |
|---|---|---|---|
| Treatment | | Docetaxel | Docetaxel |
| Groups | Parameters | n = 46 | n = 47 |
| Subsequent | Median | N/A - not yet | 12.6 (10.4-17.8) |
| IO | Months (95% Cl) | reached (15.2-N/A) | |
| | HR (95% Cl) | 0.46 (0.24-0.79) | |
| | P-value | 0.006 | |
| No | Median | 9.2 (7.2-11.0) | 10.2 (8.9-11.9) |
| Subsequent | Months (95% Cl) | | |

| | | |
|---|---|---|
| IO | HR (95% Cl) | 1.16 (0.94-1.42) |
| | P-value | 0.172 |

Within the subsequent IO groups, the particular immunotherapy agents of the “first subsequent 10” were also identified. Within the 46 patients treated with bavituximab (and docetaxel) and subsequent IO, the immunotherapy agents are shown in Table 17, all of which are checkpoint inhibitor antibodies (immune checkpoint inhibitors) in the form of a blocking antibody that binds to CTLA-4, PD-1 or PD-L1. In particular, the blocking antibodies used were tremelimumab, a blocking antibody that binds to CTLA-4; nivolumab, a blocking antibody that binds to PD-1; and durvalumab (MEDI4736), a blocking antibody that binds to PD-L1. In summary, 42/46 bavituximab patients subsequently received nivolumab; two received durvalumab monotherapy and two received tremelimumab+durvalumab (Table 17).

TABLE 17
Bavituximab and Subsequent Immunotherapeutic Agents
Bavituximab (+Docetaxel)

| Subsequent IO | Number of Patients |
|---|---|
| Durvalumab (MEDI4736) | 4 |
| Nivolumab (Opdivo ®) | 41 |
| Tremelimumab | 2 |
| Nivolumab plus IL-10 | 1 |

It will be noted that four patients received more than one IO agent, i.e., their “first subsequent IO” therapy was itself an “IO combination”, i.e., a first and second checkpoint inhibitor antibody. Therefore, in the “ITT” (Intent to Treat) analysis, there are 46 patients treated with bavituximab who received a first subsequent IO, but there are 48 subsequent IO agents in Table 17. This is because two patients received an “IO doublet”. Overall, four patients received more than one subsequent IO, and each of these received a doublet of MEDI4736 (durvalumab) and tremelimumab. Out of these four subjects, two were in the bavituximab arm and two were in the placebo arm.

Within the 93 patients receiving subsequent IO, patients with prior treatment of docetaxel alone (placebo) also received tremelimumab, nivolumab or durvalumab (MEDI4736). In addition, two patients in the placebo arm received pembrolizumab (formerly MK-3475) and one patient in the placebo arm received REGN2810, which are both blocking antibodies that bind to PD-1. Overall, the first subsequent IO in the 47 patients in the placebo arm was: tremelimumab (3), nivolumab (40), durvalumab (3), pembrolizumab (2) and REGN2810 (1), which is a total of 49 agents in 47 patients, with two patients receiving an IO doublet of durvalumab (MEDI4736)-tremelimumab. That is, 40/47 patients in the control arm subsequently received nivolumab; one received durvalumab monotherapy; one received tremelimumab monotherapy; two received tremelimumab+durvalumab; two received pembrolizumab and one received REGN2810.

FIG. 26 shows the survival benefit of initial treatment with bavituximab prior to subsequent IO in terms of time since randomization. The survival benefit of initial bavituximab treatment prior to subsequent IO is even more pronounced when measured as the time since the first subsequent IO treatment. In this context, for patients receiving subsequent IO, mOS has yet to be reached for the bavituximab and docetaxel group (95% CI, 10.2-NA), whereas it was only 6.2 months for the docetaxel alone group (95% CI, 3.9-8.7); HR=0.42 and p=0.002.

In conclusion, the data in the present example show, for the first time, that bavituximab enhances the activity of immunotherapy agents in human patients. These results therefore strongly support the ongoing and future treatment of cancer patients with bavituximab in combination with immunotherapy agents, particularly immune checkpoint inhibitors.

### Example XX

### β2GPI Biomarker Analyses For Bavituximab and Subsequent Immunotherapy

As shown in Example XIX, patients treated with bavituximab (plus docetaxel) and subsequent IO have a markedly better mOS than patients treated with docetaxel alone and subsequent IO. The present example further validates the use of functional β2GPI as a bavituximab biomarker, showing that the same levels of functional β2GPI also correlate with successful treatment by bavituximab in combination with immunotherapy.

Using the assay of Example XVI, functional β2GPI levels of 200 μg/ml or higher are shown to correlate with successful bavituximab treatment, including in the Phase III trial (Example XVII). Based on the same cut-off of “high β2GPI” as being pre-treatment levels of functional β2GPI of equal to or higher than 200 μg/ml (≥200 μg/ml), it was again determined that β2GPI≥200 μg/ml correlated with increased overall survival in patients treated with bavituximab and subsequent IO, but not in control patients who received subsequent IO (FIG. 27).

In particular, for patients with functional β2GPI of equal to or higher than 200 μg/ml, mOS has yet to be reached for patients treated with bavituximab and subsequent IO, whereas mOS was 12.3 months (10.2-17.6) for patients treated with docetaxel and subsequent IO (FIG. 27; p=0.002). As predicted by the data in Example XVII, in patients without subsequent IO, β2GPI≥200 μg/ml still trended with increased overall survival in patients treated with bavituximab (10.5 months), as compared to control (9.2 months), although the separation of the curves is not as pronounced as observed for the subsequent IO patients (FIG. 27). In contrast to bavituximab treatment, there is a trend for patients in the control arm to survive longer when β2GPI is less than 200 μg/ml, both for those with subsequent IO and without subsequent IO. Detailed analyses of the data for the β2GPI less than 200 μg/ml group are hampered by the relatively small number of patients treated with bavituximab (n=12) and placebo (n=19) and subsequent IO in the β2GPI<200 μg/ml group.

These clinical data therefore show that functional β2GPI≥200 μg/ml is a biomarker for successful treatment with bavituximab in combination with immunotherapy, particularly in combination with immune checkpoint inhibitors such as tremelimumab, nivolumab, pembrolizumab, durvalumab and atezolizumab.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are referenced.
Agostinis et al., “In vivo distribution of β2GPI under pathophysiologic conditions”, Blood, 118(15):4231-4238, 2011.
An et al., “Exosomes serve as tumour markers for personalized diagnostics owing to their important role in cancer metastasis”, J. Extracell. Vesicles, 4:27522, 2015; http://dx.doi.org/10.3402/jev.v4.27522.
Balasubramanian et al., “Estimation of plasma beta-2-glycoprotein levels by competitive ELISA”, Thromb. Res., 92:91-97, 1998.
Beck et al., “Combination of a monoclonal anti-phosphatidylserine antibody with gemcitabine strongly inhibits the growth and metastasis of orthotopic pancreatic tumors in mice”, Int. J. Cancer, 118:2639-2643, 2006.
Best, “Viruses play dead to TAMe interferon responses”, Cell Host & Microbe, 14(2):117-8, 2013.
Bevers, Zwaal, Willems, “The effect of phospholipids on the formation of immune complexes between autoantibodies and β₂-glycoprotein I or prothrombin”,Clin. Immunol., 112:150-160, 2004.
Bevers et al., “Quantitative determination of the binding of β₂-glycoprotein I and prothrombin to phosphatidylserine-exposing blood platelets”, Biochem. 1, 386:271-279, 2005.
Bhattacharyya et al., “Enveloped viruses disable innate immune responses in dendritic cells by direct activation of TAM receptors”, Cell Host & Microbe, 14(2):136-147, 2013.
Birge et al., “Phosphatidylserine is a global immunosuppressive signal in efferocytosis, infectious disease, and cancer”, Cell Death Differ., 23(6):962-78, 2016.
Brahmer et al., “Nivolumab versus docetaxel in advanced squamous-cell non-small-cell lung cancer”, N. Engl. J. Med., 373(2):123-135, 2015.
Chalasani et al., “A Phase I Clinical Trial of Bavituximab and Paclitaxel in Patients with HER2 Negative Metastatic Breast Cancer”, Cancer Medicine, 4(7):1051-1059, 2015.
Chen et al., “Phosphatidylserine Vesicles Enable Efficient En Bloc Transmission of Enteroviruses”, Cell, 160:619-630, 2015.
Cheng et al., “Antibody-Mediated Blockade of Phosphatidylserine Enhances the Antitumor Effect of Sorafenib in Hepatocellular Carcinomas Xenografts”, Ann. Surg. Oncol., 5107-5, DOI 10.1245/s10434-016-5107-5, 2016.
Clayson et al., “Release of Simian Virus 40 Virions from Epithelial Cells is Polarized and Occurs without Cell Lysis”, J. Virology, 63(5):2278-2288, 1989.
Czuczman et al., “Listeria monocytogenes exploits efferocytosis to promote cell-to-cell spread”, Nature, 509:230-234, 2014.
DaMatta et al., “Trypanosoma cruzi exposes phosphatidylserine as an evasion mechanism”, FEMS Microbiol. Lett., 266:29-33, 2007.
de Laat, Derksen, Urbanus, de Groot, “IgG antibodies that recognize epitope Gly40-Arg43 in domain I of β₂-glycoprotein I cause LAC, and their presence correlates strongly with thrombosis”, Blood, 105(4):1540-5, 2005.
de Laat, Derksen, van Lummel, Pennings, de Groot, “Pathogenic anti-β₂-glycoprotein I antibodies recognize domain I of β₂-glycoprotein I only after a conformational change”, Blood, 107(5):1916-24, 2006.
DeRose et al., “Development of bavituximab, a vascular targeting agent with immune-modulating properties, for lung cancer treatment”, Immunotherapy, 3(8):933-944, 2011.
Digumarti et al., “Bavituximab Plus Paclitaxel and Carboplatin for the Treatment of Advanced Non-Small-Cell Lung Cancer”, Lung Cancer, 86:231-236, 2014.
Eda & Sherman, “Cytoadherence of Malaria-Infected Red Blood Cells Involves Exposure of Phosphatidylserine”, Cell Physiol. Biochem., 12:373-384, 2002.
Fehrenbacher et al., “Atezolizumab versus docetaxel for patients with previously treated non-small-cell lung cancer (POPLAR): a multicentre, open-label, phase 2 randomised controlled trial”, The Lancet, 387(10030):1837-1846, 2016.
Francis et al., “Mycobacterium tuberculosis ESAT-6 is a leukocidin causing Ca2+ influx, necrosis and neutrophil extracellular trap formation”, Cell Death and Disease, 5:e1474; doi:10.1038/cddis.2014.394, 2014.
Freimark et al., “Antibody-Mediated Phosphatidylserine Blockade Enhances the Antitumor Responses to CTLA-4 and PD-1 Antibodies in Melanoma”, Cancer Immunol. Res., 4(6):531-40, 2016.
Galli et al., “Anticardiolipin antibodies (ACA) directed not to cardiolipin but to a plasma protein cofactor”, Lancet, 335(8705):1544-1547, 1990.
Garon et al., “Pembrolizumab for the treatment of non-small-cell lung cancer”, N. Engl. J. Med., 372(21):2018-2028, 2015.
Gerber et al., “Phase I Safety and Pharmacokinetic Study of Bavituximab, a Chimeric Phosphatidylserine-Targeting Monoclonal Antibody, in Patients with Advanced Solid Tumors”, Clin. Cancer Res., 17(21):1-9, 2011.
Gerber et al., “Docetaxel Combined with Bavituximab in Previously Treated, Advanced Nonsquamous Non-Small-Cell Lung Cancer”, Clinical Lung Cancer, 17(3):169-176, 2016.
Gong et al., “Measuring Response to Therapy by Near-Infrared Imaging of Tumors Using a Phosphatidylserine-Targeting Antibody Fragment”, Molecular Imaging, 12(4):244-256, 2013.
Goth & Stephens, “Rapid, Transient Phosphatidylserine Externalization Induced in Host Cells by Infection with Chlamydia spp”, Infect. Immun., 69(2):1109-1119, 2001.
Gray et al., “Phosphatidylserine-targeting antibodies augment the anti-tumorigenic activity of anti-PD-1 therapy by enhancing immune activation and downregulating pro-oncogenic factors induced by T-cell checkpoint inhibition in murine triple-negative breast cancers”, Breast Cancer Research, 18(1):50, DOI 10.1186/s13058-016-0708-2, 2016a.
Gray et al., “LAG3 is an immunotherapeutic target in murine triple negative breast cancers whose activity is significantly enhanced in combination with phosphatidylserine targeting antibodies”, Poster B019, CRI-CIMT-EATI-AACR International Cancer Immunotherapy Conference, New York, N.Y., September 25-28, 2016b.
Gregorc et al., “Predictive value of a proteomic signature in patients with non-small-cell lung cancer treated with second-line erlotinib or chemotherapy (PROSE): a biomarker-stratified, randomised phase 3 trial”, Lancet Oncology, 15(7):713-721, 2014.
Hägele et al., “Legionella pneumophila kills human phagocytes but not protozoan host cells by inducing apoptotic cell death”, FEMS Microbiol. Lett., 169(1):51-58, 1998.
He et al., “Radiation-enhanced vascular targeting of human lung cancers in mice with a monoclonal antibody that binds anionic phospholipids”, Clin. Cancer Res., 13(17):5211-5218, 2007.
He et al., “Antiphosphatidylserine antibody combined with irradiation damages tumor blood vessels and induces tumor immunity in a rat model of glioblastoma”, Clin. Cancer Res., 15(22):6871-80, 2009.
Hogg et al., “Retargeting Adenoviral Vectors to Improve Gene Transfer into Tumors”, Cancer Gene Therapy, 18:275-287, 2011.
Hotchkiss et al., “Inhibition of endothelial cell function in vitro and angiogenesis in vivo by docetaxel (Taxotere): association with impaired repositioning of the microtubule organizing center”, Mol. Cancer Ther., 1 (13):1191-200, 2002.
Huang, Bennett, Thorpe, “A monoclonal antibody that binds anionic phospholipids on tumor blood vessels enhances the antitumor effect of docetaxel on human breast tumors in mice”, Cancer Res., 65(10):4408-4416, 2005.
Hunt, Simpson, Krilis, “Identification of a region of β₂-glycoprotein I critical for lipid-binding and anticardiolipin antibody cofactor activity”, Proc. Natl. Acad. Sci. USA, 90:2141-2145, 1993.
Hunt and Krilis, “The fifth domain of β₂-glycoprotein I contains a phospholipid-binding site (Cys281-Cys288) and a region recognized by anticardiolipin antibodies”, J. Immunol., 152:653-659, 1994.
Ioannou et al., “Binding of antiphospholipid antibodies to discontinuous epitopes on domain I of human β₂-glycoprotein I: mutation studies including residues R39 to R43”, Arthritis Rheum., 56(1):280-90, 2007.
Izquierdo-Useros et al., “HIV and mature dendritic cells: Trojan exosomes riding the Trojan horse?”, PLoS Pathog, 6(3):e1000740, 2010.
Jennewein et al., “Vascular Imaging of Solid Tumors in Rats with a Radioactive Arsenic-Labeled Antibody that Binds Exposed Phosphatidylserine”, Clin. Cancer Res., 14(5):1377-1385, 2008.
Jemielity et al., “TIM-Family Proteins Promote Infection of Multiple Enveloped Viruses through Virion-Associated Phosphatidylserine”, PLoS Pathogens, 9(3):e1003232; 2013.
Judy et al., “Vascular Endothelial-Targeted Therapy Combined with Cytotoxic Chemotherapy Induces Inflammatory Intratumoral Infiltrates and Inhibits Tumor Relapses after Surgery”, Neoplasia, 14:352-359, 2012.
Kabat et al., “Sequences of Proteins of Immunological Interest” 5th Ed. Public Health Service, National Institutes of Health, Bethesda, MD, 1991, pp 647-669 in particular.
Kamboh et al., “Genetic Studies of Human Apolipoproteins. IV. Structural Heterogeneity of Apolipoprotein H (β2-Glycoprotein I)”, Am. J. Hum. Genet., 42:452-457, 1988.
Kennedy et al., “Attenuating a sickle cell crisis with annexin V”, Medical Hypotheses, http://dx.doi.org/10.1016/j.mehy.2015.01.037, 2015.
Klein & Moeschberger, “Survival Analysis. Techniques for censored and truncated data”, 2^{nd} Edition, New York, 2003 (ISBN-10: 038795399X; ISBN-13: 978-0387953991).
Kogure et al., “Temporary membrane distortion of vascular smooth muscle cells is responsible for their apoptosis induced by platelet-activating factor-like oxidized phospholipids and their degradation product, lysophosphatidylcholine”, Chemistry and Physics of Lipids, 126:29-3 8, 2003.
Larson et al., “Customization, Scale-Up and Qualification of an Antibody-dependent Cell-mediated Cytotoxicity (ADCC) Bioassay”, IBC's 23^{rd} International Intensive Symposium Development, Validation and Maintenance of Biological Assays Conference, Seattle, Wash., May 14-16, 2013; Poster Board #7.
Li et al., “Phosphatidylserine (PS) is Exposed in Choroidal Neovascular Endothelium: PS-Targeting Antibodies Inhibit Choroidal Angiogenesis In vivo and Ex Vivo”, Invest. Ophthalmol. Vis. Sci., 56:7137-7145, 2015.
Liang et al., “Targeting Mutant P53 Protein and the Tumor Vasculature: An Effective Combination Therapy for Advanced Breast Tumors”, Breast Cancer Res. Treat., 125:407-420, 2011.
Lonsdale et al., “Phosphatidylserine as a Therapeutic Target for the treatment of Francisella tularensis and Yersinia pestis infections”, Chemical & Biological Defense Science & Technology Conference, 2011 Las Vegas, Nev. Abstract W15-048.
Luster et al., “Plasma Protein β₂-glycoprotein 1 Mediates Interaction between the Anti-tumor Monoclonal Antibody 3G4 and Anionic Phospholipids on Endothelial Cells”, J. Biol. Chem., 281(40):29863-29871, 2006.
Mallat et al., “Shed Membrane Microparticles With Procoagulant Potential in Human Atherosclerotic Plaques”, Circulation, 99:348-353, 1999.
Marconescu & Thorpe, “Coincident Exposure of Phsophatidylethanolamine and Anionic Phospholipids on the Surface of Irradiated Cells”, Biochemica et Biophysica Acta, 1778(10):2217-2224, 2008.
McNeil, Simpson, Chesterman, Krilis, “Anti-phospholipid antibodies are directed against a complex antigen that includes a lipid-binding inhibitor of coagulation: β₂-glycoprotein I (apolipoprotein H)”, Proc. Natl. Acad. Sci. USA, 87(11):4120-4124, 1990.
Meckes et al., “Human tumor virus utilizes exosomes for intercellular communication”, Proc. Natl. Acad. Sci. USA, 107(47):20370-20375, 2010.
Meckes and Raab-Traub, “Microvesicles and Viral Infection”, J. Virology, 85(24):12844-12854, 2011.
Mehdi et al., “Genetic variation in the apolipoprotein H (β2-glycoprotein I) gene affects plasma apolipoprotein H concentrations”, Hum. Genet., 105:6371, 1999.
Meertens et al., “The TIM and TAM families of phosphatidylserine receptors mediate dengue virus entry”, Cell Host & Microbe, 12(4):544-557, 2012.
Mercer and Helenius, “Vaccinia virus uses macropinocytosis and apoptotic mimicry to enter host cells”, Science, 320:531-535, 2008.
Miyakis et al., “β2-glycoprotein I—function in health and disease”, Thromb. Res., 114:335-346, 2004.
Moller-Tank & Maury, “Phosphatidylserine receptors: Enhancers of enveloped virus entry and infection”, Virology, 468-470 (2014) 565-580, 2014.
Moody et al., “Anti-phospholipid human monoclonal antibodies inhibit CCR5-tropic HIV-1 and induce β-chemokines”, J. Exp. Med., 207(4):763-776, 2010.
Morizono et al., “The soluble serum protein Gas6 bridges virion envelope phosphatidylserine C254 to the TAM receptor tyrosine kinase Axl to mediate viral entry”, Cell Host Microbe, 9:286-298. 2011.
Murata-Kamiya et al., “Helicobacter pylori Exploits Host Membrane Phosphatidylserine for Delivery, Localization, and Pathophysiological Action of the CagA Oncoprotein”, Cell Host Microbe, 7:399-411, 2010.
Palmero et al., “Final Clinical Results from SUNRISE: A Phase III, Randomized, Double-Blind, Placebo-Controlled Multicenter Trial of Bavituximab Plus Docetaxel in Patients with Previously Treated Stage IIIb/IV Nonsquamous Non-Small Cell Lung Cancer”, Annals Oncol., 28(Suppl. 5):487, Abstract 1364P, 2017.
Pattanapanyasat et al., “Febrile temperature but not proinflammatory cytokines promotes phosphatidylserine expression on Plasmodium falciparum malaria-infected red blood cells during parasite maturation”, Cytometry, Part A, 77A:515-523, 2010.
Petersen & Krogfelt, “Helicobacter pylori: an invading microorganism? A review”, FEMS Immunol. Med. Microbiol., 36:117-126, 2003.
Polz, Wurm, Kostner, “Investigations on β2-glycoprotein-I in the rat—isolation from serum and demonstration in lipoprotein density fractions”, Int. J. Biochem., 11:265-270, 1980.
Prakasam and Thiagarajan, “β2-Glycoprotein I—A Protein in Search of Function”, In Antiphospholipid Syndrome, Ed. Alena Bulikova, ISBN: 978-953-51-0526-8, InTech, Available from: http://www.intechopen.com/books/antiphospholipid-syndrome/beta2-glycoprotein-i-in-search-of-function, 2012.
Ran et al., “Antitumor effects of a monoclonal antibody that binds anionic phospholipids on the surface of tumor blood vessels in mice”, Clin. Cancer Res., 11:1551-1562, 2005.
Sabatier et al., “Type 1 And Type 2 Diabetic Patients Display Different Patterns of Cellular Microparticles”, Diabetes, 51:2840-2845, 2002.
Saha et al., “An Orthotopic Lung Tumor Model for Image-Guided Microirradiation in Rats”, Radiat. Res., 174:62-71, 2010.
Schubert-Unkmeir et al., “Gene Expression Pattern in Human Brain Endothelial Cells in Response to Neisseria meningitidis”, Infect. Immun., 75(2):899-914, 2007.
Seabra et al., “Toxoplasma gondii exposes phosphatidylserine inducing a TGF-beta1 autocrine effect orchestrating macrophage evasion”, Biochem. Biophys. Res. Comm., 324(2):744-752, 2004.
Sheng et al., “Impaired Thrombin Generation in β2-Glycoprotein I Null Mice”, J. Biol. Chem., 276(17):13817-13821, 2011.
Sims et al., “Neural stem cell-derived exosomes mediate viral entry”, Int. J. Nanomedicine, 9:4893-4897, 2014.
Soares et al., “Targeting inside-out phosphatidylserine as a therapeutic strategy for viral diseases”, Nature Medicine, 14(12):1357-1362, 2008.
Souza et al., “Microparticles: markers and mediators of sepsis-induced microvascular dysfunction, immunosuppression, and AKI”, Kidney Int., doi: 10.1038/ki.2015.26, 2015.
Stafford & Thorpe, “Increased Exposure of Phosphatidylethanolamine on the Surface of Tumor Vascular Endothelium”, Neoplasia, 13:299-308, 2011.
Stafford et al., “Highly Specific PET Imaging of Prostate Tumors in Mice with an Iodine-124-Labeled Antibody Fragment that Targets Phosphatidylserine”, PLoS ONE, 8(12):e84864, 2013.
Steinkasserer et al., “Complete nucleotide and deduced amino acid sequence of human β2-glycoprotein I”, Biochem. J., 277:387-391, 1991.
Takeuchi et al., “Coagulation and fibrinolytic activities in 2 siblings with β2-glycoprotein I deficiency”, Blood, 96:1594-1595, 2000.
U.S. Patent Application No. US 2016/0009805 A1.
U.S. Pat. No. 5,019,368
U.S. Pat. No. 5,882,626
U.S. Pat. No. 7,247,303
U.S. Pat. No. 7,422,738
U.S. Pat. No. 7,455,833
U.S. Pat. No. 7,572,448
U.S. Pat. No. 7,611,704
U.S. Pat. No. 7,790,860
U.S. Pat. No. 7,906,115
U.S. Pat. No. 8,486,391
U.S. Pat. No. 8,956,616
van der Kleij et al., “A Novel Host-Parasite Lipid Cross-talk: schistosomal lyso-phosphatidylserine activates toll-like receptor 2 and affects immune polarization”, J. Biol. Chem., 277(50):48122-48129, 2002.
Walker et al., “Cytomegalovirus-infected human endothelial cells can stimulate allogeneic CD4+ memory T cells by releasing antigenic exosomes”' J. Immunol., 182(3):1548-1559, 2009.
Wanderley et al., “Cooperation between apoptotic and viable metacyclics enhances the pathogenesis of leishmaniasis”, PLoS One, 4(5):e5733, 2009.
Wanderley et al., “Phosphatidylserine exposure on the surface of Leishmania amazonensis amastigotes modulates in vivo infection and dendritic cell function”, Parasite Immunology, 35:109-119, 2013.
Wandler et al., “A Greasy Foothold for Helicobacter pylori”, Cell Host Microbe, 7:338-339, 2010.
Weihua et al., “Apoptotic Cells Initiate Endothelial Cell Sprouting via Electrostatic Signaling”, Cancer Res., 65(24): 11529-11535, 2005.
Willems, Janssen, Pelsers et al., “Role of divalency in the high-affinity binding of anticardiolipin antibody-β2-glycoprotein I complexes to lipid membranes”, Biochemistry, 35:13833-13842, 1996.
Wurm, “β2-glycoprotein-I (apolipoprotein H) interactions with phospholipid vesicles”, Int. J. Biochem., 16:511-15, 1984.
Yin et al., “Phosphatidylserine-targeting antibody induces M1 macrophage polarization and promotes myeloid-derived suppressor cell differentiation”, Cancer Immunol. Res., 1(4):256-268, 2013.
Yuyama et al., “Sphingolipid-modulated Exosome Secretion Promotes Clearance of Amyloid-β by Microglia”, J. Biol. Chem., 287(14):10977-10989, 2012.
Zandbergen et al., “Leishmania disease development depends on the presence of apoptotic promastigotes in the virulent inoculum”, Proc. Natl. Acad. Sci. USA., 103(37):13837-13842, 2006.
Zhang et al., “Phosphatidylserine-Targeted Bimodal Liposomal Nanoparticles for in vivo Imaging of Breast Cancer in Mice”, J. Control. Release, 183:114-123, 2014.
Zhao et al., “Near-Infrared Optical Imaging of Exposed Phosphatidylserine in a Mouse Glioma Model”, Translational Oncology, 4:355-364, 2011.
Zhou et al., “Phosphatidylserine-Targeted Molecular Imaging of Tumor Vasculature by Magnetic Resonance Imaging”, J. Biomed. Nanotechnol., 10:1-10, 2014.

1-40. (canceled)

41. A method for treating a tumor in a human subject in need thereof, comprising
administering to the subject a therapeutically effective amount of
(i) a phosphatidylserine (PS)-targeting antibody, and
(ii) an antibody that specifically binds to programmed cell death protein 1 (PD-1),
wherein the subject is identified as having functional β2-glycoprotein 1 (β2GP1) present at a concentration of equal to or greater than 200 μg/ml in a blood sample from the subject.

42. A method for treating a tumor in a human subject in need thereof, comprising
(a) determining the expression level of functional β2GP1 in a blood sample from the subject; and,
(b) administering to the subject a therapeutically effective amount of
(i) a PS-targeting antibody, and
(ii) an antibody that specifically binds to PD-1,
if the blood sample is identified as having functional β2GP1 present at a concentration equal to or greater than 200 μg/ml.

43. A method for treating a tumor in a human subject in need thereof, comprising
administering to the subject a therapeutically effective amount of
(i) a PS-targeting antibody and
(ii) an antibody that specifically binds to PD-1 which comprises pembrolizumab or an antigen binding portion thereof,
if a blood sample from the subject is identified as having functional β2GP1 present at a concentration equal to or greater than 200 μg/ml.

44. The method of claim 43, wherein the blood concentration of functional β2GP1 is between 200 μg/ml and 290 μg/ml.

45. The method of claim 43, wherein the blood sample is a plasma sample.

46. The method of claim 43, wherein the blood sample is a serum sample.

47. The method of claim 43, wherein functional β2GP1 is measured in an assay comprising:
(a) coating an ELISA plate with PS to prepare a PS-coated ELISA plate;
(b) adding PS-targeting antibody and the blood sample to the PS-coated ELISA plate under conditions effective to allow binding of β2GP1 in the blood sample to both the PS-targeting antibody and the PS-coated ELISA plate; and
(c) detecting the binding of PS-targeting antibody and β2GP1 to the PS-coated ELISA plate, thereby measuring functional β2GP1 in the blood sample.

48. The method of claim 47, wherein the PS-targeting antibody is attached to a detectable agent that produces a detectable signal, and wherein the binding of PS-targeting antibody and β2GP1 to the PS-coated ELISA plate is detected and measured by detecting and measuring the detectable signal.

49. The method of claim 47, wherein the PS-targeting antibody is added to the PS-coated ELISA plate prior to adding the sample containing β2GP1.

50. The method of claim 43, wherein the PS-targeting antibody comprises bavituximab or an antigen binding portion thereof.

51. The method of claim 43, wherein the PS-targeting antibody is administered to the subject prior to, concurrently with, or after the administration of pembrolizumab or an antigen binding portion thereof.

52. The method of claim 43, wherein the PS-targeting antibody is administered weekly or once every two, three, four, five, or six weeks.

53. The method of claim 52, wherein the PS-targeting antibody is administered weekly.

54. The method of claim 43, wherein pembrolizumab is administered weekly or once every two, three, four, five, or six weeks.

55. The method of claim 54, wherein pembrolizumab is administered once every three weeks.

56. The method of claim 43, wherein the PS-targeting antibody is administered intravenously.

57. The method of claim 43, wherein pembrolizumab is administered intravenously.

58. The method of claim 43, wherein the PS-targeting antibody is administered to the subject in an amount of about 1 mg/kg, about 2 mg/kg, or about 3 mg/kg.

59. The method of claim 43, wherein pembrolizumab is administered to the subject in an amount of about 1 mg/kg, about 2 mg/kg, or about 3 mg/kg.

60. The method of claim 43, wherein the PS-targeting antibody is administered to the subject in an amount between about 100 mg and about 500 mg per dose.

61. The method of claim 43, wherein pembrolizumab is administered to the subject in an amount between about 100 mg and about 500 mg per dose.

62. The method of claim 43, wherein the PS-targeting antibody is administered to the subject at a flat dose of about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, or about 400 mg.

63. The method of claim 43, wherein pembrolizumab is administered to the subject at a flat dose of about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, or about 400 mg.

64. The method of claim 43, wherein the tumor is a lung cancer, ovarian cancer, gastric cancer, hepatocellular carcinoma, colorectal cancer, esophageal cancer, malignant glioma, pancreatic cancer, prostate cancer, Merkel cell carcinoma, head and neck cancer, renal cell carcinoma, bladder cancer, liver cancer, non-small cell lung cancer (NSCLC), metastatic gastroesphogeal cancer, recurrent/metastatic squamous cell head and neck cancer (HNSCC), or hepatocellular carcinoma tumor.

65. The method of claim 43, wherein the subject is immunosuppressed.

66. A method for treating a tumor in a human subject in need thereof, comprising
administering to the subject a therapeutically effective amount of
(i) bavituximab or an antigen binding portion thereof, and
(ii) pembrolizumab or an antigen binding portion thereof,
if a blood sample from the subject is identified as having functional β2GP1 present at a concentration equal to or greater than 200 μg/ml.

67. The method of claim 66, wherein bavituximab is administered to the subject in an amount of about 1 mg/kg, about 2 mg/kg, or about 3 mg/kg, and pembrolizumab is administered to the subject in an amount of about 200 mg.

68. The method of claim 67, wherein bavituximab is administered to the subject weekly, and pembrolizumab is administered to the subject once every three weeks.
